(19) United States
(12) Patent Application Publication
(10) Pub. No.: US 2025/0074965 A1
(43) Pub. Date: Mar. 6, 2025

(54) ANTI-PTK7 IMMUNE CELL CANCER THERAPY

(71) Applicant: CRISPR THERAPEUTICS AG, Zug (CH)

(72) Inventors: Jonathan Alexander Terrett, Cambridge, MA (US); Jason Sagert, Cambridge, MA (US)

(21) Appl. No.: 18/947,327

(22) Filed: Nov. 14, 2024

Related U.S. Application Data
(63) Continuation of application No. 16/677,207, filed on Nov. 7, 2019, now Pat. No. 12,146,157.
(60) Provisional application No. 62/910,586, filed on Oct. 4, 2019.
(60) Provisional application No. 62/756,638, filed on Nov. 7, 2018.

(51) Int. Cl. C07K 14/705; A61K 35/17; A61K 39/00; A61P 35/00; C07K 14/725; C07K 16/40; C12N 5/0783; C12N 15/11; C12N 15/86

(52) U.S. Cl. CPC C07K 14/70521; A61K 35/17; A61K 39/4611; A61K 39/4631; A61K 39/464412; A61K 39/464462; A61P 35/00; C07K 14/7051; C07K 14/70575; C07K 16/40; C12N 5/0636; C12N 15/11; C12N 15/86; A61K 2039/505; A61K 2239/38; A61K 2239/49; A61K 2239/54; A61K 2239/55; A61K 2239/59; C07K 2317/565; C07K 2317/622; C07K 2319/02; C12N 2750/14143

(57) ABSTRACT

Provided herein, in some embodiments, are methods and compositions (e.g., cell compositions) for the treatment of cancer, such as PTK7+ malignancies.

### RELATED APPLICATIONS

This application is a continuation of U.S. patent application Ser. No. 16/677,207, filed Nov. 7, 2019, which claims the benefit of U.S. Provisional Application No. 62/756,638, filed Nov. 7, 2018, and U.S. Provisional Application No. 62/910,586, filed Oct. 4, 2019, the disclosures of which are hereby incorporated by reference in their entirety.

### INCORPORATION BY REFERENCE OF MATERIAL SUBMITTED ELECTRONICALLY

The Sequence Listing, which is a part of the present disclosure, is submitted concurrently with the specification. The name of the text file containing the Sequence Listing is “CRISP_41821_305_SequenceListing.xml”, which was created on Nov. 13, 2024 and is 152,453 bytes in size. The subject matter of the Sequence Listing is incorporated herein in its entirety by reference.

### BACKGROUND

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

### SUMMARY

Multiple tumor-associated antigen targets have been progressed into clinical trials, chosen predominantly using the logic that expression in cancer tissues should be selective over normal tissues to avoid toxicity (Morgan, R. Blood 2013; 122(2): 3392-3394). PTK7, however, does not meet this criteria due to its apparent excessive expression in normal tissues including lung, smooth muscle, stomach, kidney and bladder. Thus, PTK7 has not been considered a viable CAR T cell target. Nonetheless, the data provided herein unexpectedly demonstrate that animals do, in fact, tolerate therapy with anti-PTK7 CAR T cells, and these anti-PTK7 CAR T cells effectively and selectively kill cells expressing PTK7.

Some aspects of the present disclosure provide an engineered T cell comprising a nucleic acid encoding a chimeric antigen receptor (CAR), wherein the CAR comprise an ectodomain that binds specifically to PTK7. In some embodiments, the engineered T cell further comprises a disrupted T cell receptor alpha chain constant region (TRAC) gene. For example, the TRAC gene may be disrupted by insertion of the nucleic acid encoding a CAR. In some embodiments, the engineered T cell further comprises a disrupted beta-2-microglobulin (β2M) gene.

The ectodomain of the CAR, in some embodiments, comprises an anti-PTK7 antibody. In some embodiments, the anti-PTK7 antibody is an anti-PTK7 single-chain variable fragment (scFv). The anti-PTK7 scFv, in some embodiments, comprises an amino acid sequence of any one of SEQ ID NO: 54, 68, 75, or 82. In some embodiments, the anti-PTK7 scFv comprises a VH comprising an amino acid sequence of any one of SEQ ID NO: 55, 69, 76, or 83 and/or a VL comprising an amino acid sequence of any one of SEQ ID NO: 56, 70, 77, or 84. In some embodiments, the anti-PTK7 scFv comprises a VH comprising CDR amino acid sequences of SEQ ID NO: 57, SEQ ID NO: 58, and/or SEQ ID NO: 59; and/or the anti-PTK7 scFv comprises a VL sequence comprising CDR amino acid sequences of SEQ ID NO: 60, SEQ ID NO: 61, and/or SEQ ID NO: 62. In some embodiments, the anti-PTK7 scFv comprises a VH comprising CDR amino acid sequences of SEQ ID NO: 85, SEQ ID NO: 86, and/or SEQ ID NO: 87; and/or the anti-PTK7 scFv comprises a VL sequence comprising CDR amino acid sequences of SEQ ID NO: 88, SEQ ID NO: 89, and/or SEQ ID NO: 90.

The CAR, in some embodiments, comprises a CD3ζ cytoplasmic signaling domain. In some embodiments, the CAR comprises a CD28 co-stimulatory domain or a 41BB co-stimulatory domain.

In some embodiments, the TRAC gene comprises the nucleotide sequence encoding the left homology arm (LHA) and/or right homology arm (RHA) within of any one of SEQ ID NOs: 63, 64, 71, 78, or 91 or the nucleotide sequence of SEQ ID NO: 92 or 100, and/or wherein the CAR is encoded by the nucleotide sequence of any one of SEQ ID NOs: 49, 51, 65, 72, 79, or 112. In some embodiments, the disrupted β2M gene comprises at least one nucleotide sequence selected from any one of SEQ ID NOs: 9-14.

Also provided herein, in some aspects, is an engineered T cell comprising: (i) a disrupted TRAC gene; (ii) a disrupted β2M gene; and (iii) a nucleic acid encoding a CAR comprising an anti-PTK7 antigen-binding fragment.

Also provided herein, in some aspects, is a population of cells comprising engineered T cells, wherein the engineered T cells comprise: (i) a disrupted TRAC gene; (ii) a disrupted β2M gene; and (iii) a nucleic acid encoding a CAR comprising an anti-PTK7 antigen-binding fragment. In other aspects, provided herein is A population of cells comprising engineered T cells, wherein the engineered T cells comprise: (i) a disrupted TRAC gene, wherein the disrupted TRAC gene comprises a nucleic acid encoding a CAR, wherein the nucleic acid sequence is at least 90% identical to SEQ ID NOs: 49, 51, 65, 72, 79, or 112 and encodes the CAR of SEQ ID NOs: 50, 52, 66, 73, or 80; and (ii) a disrupted β2M gene.

Also provided herein, in some aspects, is a population of engineered T cells (e.g., comprising a nucleic acid encoding an anti-PTK7 CAR), wherein at least 25% or at least 50% of engineered T cells of the population express the CAR. For example, at least 70% of engineered T cells of the population express the CAR.

In some embodiments, at least 25% of engineered T cells of the population express the CAR following at least 7 or at least 14 days of in vitro proliferation.

In some embodiments, at least 50% of engineered T cells of the population do not express a detectable level of T cell receptor (TCR) protein. For example, at least 90% of engineered T cells of the population may not express a detectable level of TCR protein.

In some embodiments, at least 50% of engineered T cells of the population do not express a detectable level of β2M protein. For example, at least 70% of engineered T cells of the population may not express a detectable level of β2M protein.

In some embodiments, engineered T cells of the population, when co-cultured in vitro with a population of cancer cells that express PTK7, induce cell lysis of at least 50% of the cancer cells of the population. For example, engineered T cells of the population may induce cell lysis of at least 70%, at least 80%, or at least 90% of the cancer cells of the population. In some embodiments, engineered T cells of the population, when co-cultured in vitro with a population of cancer cells that express PTK7, induce cell lysis of at least 10%, at least 25%, or at least 50% of the cancer cells of the population. In some embodiments, engineered T cells of the population, when co-cultured in vitro with a population of cancer cells, secrete IFNγ. In some embodiments, the ratio of engineered T cells to cancer cells is 1:1 to 2:1. The cancer cells may be, for example, sarcoma cells or breast cancer cells. Other cancer cells may be targeted. In some embodiments, the cancer cells may be breast cancer cells, ovarian cancer cells, small cell lung cancer cells, and/or colon cancer cells

In some embodiments, proliferative capacity of engineered T cells of the population is within 10% of proliferative capacity of control cells. In still other embodiments, the population of T cells, when administered in vivo to a subject, does not induce toxicity in the subject.

Other aspects of the present disclosure provide a method that comprises administering the population of engineered T cells as described herein. In some embodiments, percent body weight of the subject, following 5-10 days of administration, is within 10% of initial body weight of the subject, wherein initial body weight of the subject is body weight of the subject at the time of administration. In some embodiments, the subject is a human subject. In some embodiments, the subject has a cancer. The cancer may express PTK7, for example. In various embodiments, the cancer is selected from the group consisting of: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, and intrahepatic biliary cancer.

Further aspects of the present disclosure provide a method for producing an engineered T cell, the method comprising (a) delivering to a T cell a RNA-guided nuclease, a gRNA targeting a TRAC gene, and a vector comprising a donor template that comprises a nucleic acid encoding a CAR that comprise an ectodomain that binds specifically to PTK7, wherein the nucleic acid encoding the CAR is flanked by left and right homology arms to the TRAC gene, and (b) producing an engineered T cell. In some embodiments, the gRNA targeting the TRAC gene comprises the nucleotide sequence of SEQ ID NO: 18 or 19, or targets the nucleotide sequence of SEQ ID NO: 40. In one related embodiment, the method is provided wherein the nucleic acid encoding the CAR is flanked by left and right homology arms to the TRAC gene.

In some embodiments, the method further comprises delivering to the T cell a gRNA targeting the β2M gene. In some embodiments, the gRNA targeting the β2M gene comprises the nucleotide sequence of SEQ ID NO: 20 or 21, or targets the nucleotide sequence of SEQ ID NO: 41.

In some embodiments, the RNA-guided nuclease is a Cas9 nuclease, optionally a S. pyogenes Cas9 nuclease. In some embodiments, the ectodomain of the CAR is an anti-PTK7 antibody, optionally an anti-PTK7 single-chain variable fragment (scFv).

In some embodiments, the donor template comprises the nucleotide sequence of any one of SEQ ID NOs: 63, 64, 71, 78, or 91.

In some embodiments, the CAR comprises the nucleotide sequence encoding any one of SEQ ID NOs: 50, 52, 66, 73, or 80.

In some embodiments, the method of producing is provided wherein the anti-PTK7 scFv comprises the same heavy chain variable domain (VH) complementarity determining regions (CDRs) and the same light chain variable domain (VL) CDRs as a reference antibody, wherein the reference antibody comprises (i) a VH set forth as SEQ ID NO: 55 and VL set forth as SEQ ID NO: 56, (ii) a VH set forth as SEQ ID NO: 69 and a VL set forth as SEQ ID NO: 70, (iii) a VH set forth as SEQ ID NO: 76 and a VL set forth as SEQ ID NO: 77, or (iv) a VH set forth as SEQ ID NO: 83 and a VL set forth as SEQ ID NO: 84. In one embodiment, the method is provided wherein the anti-PTK7 scFv comprises the same VH and VL chains as the reference antibody. In still another embodiment, the anti-PTK7 scFv comprises the amino acid sequence of any one of SEQ ID NOs: 54, 68, 75, or 82.

In one embodiment, an aforementioned method of producing is provided wherein the CAR comprises a CD28 co-stimulatory domain or a 41BB co-stimulatory domain. In a related embodiment, the CAR further comprises a CD3ζ cytoplasmic signaling domain.

In some embodiments, an aforementioned method of producing is provided wherein the donor template comprises the nucleotide sequence of any one of SEQ ID NOs: 63, 64, 71, 78, or 91. In still other embodiments, the CAR is encoded by a nucleotide sequence of any one of SEQ ID NOs: 49, 51, 65, 72, 79, or 112.

### BRIEF DESCRIPTION OF THE DRAWINGS

FIG. 1 shows PTK7 expression normal human tissues.

FIGS. 2A and 2B show PTK7 expression in human diseased and normal tissues.

FIG. 3 shows PTK7 patient prevalence by immunohistochemistry (IHC) in solid tumors.

FIG. 4 shows binding affinity of PTK7/CTX181 Ab in human and murine cell lines.

FIGS. 5A and 5B show PTK7 expression in frozen normal tissue panels (FDA standard) from mouse (FIG. 5A) and human (FIG. 5B).

FIG. 6 shows PTK7 expression in frozen murine embryonic development array.

FIG. 7 includes graphs showing highly efficient multiple gene editing in TRAC⁻/β2M⁻/anti-PTK7 CAR⁺ T cells. Editing phenotypes as measured by FACS (left graph) and anti-PTK7 CAR⁺ expression as measured by immunohistochemistry (right graph) are shown for four different anti-PTK7 CAR⁺ constructs (PTK7-4, PTK7-7, PTK7-13, and PTK7-17).

FIG. 8 includes a graph showing that the PTK7 CAR T cell with CD28 co-stimulatory domain (PTK7-4) was more efficacious than the PTK7 CAR T cell with 4-1BB co-stimulatory domain (PTK7-4b).

FIGS. 9A-9C include graphs showing cell-killing effects of TRAC⁻/β2M⁻/anti-PTK7 CAR⁺ T cells against adherent sarcoma cell lines A-204 (FIG. 9A) and Saos-2 (FIG. 9B) and the breast cancer cell line MCF7 (FIG. 9C). Cell ratios (CAR T cell:target cancer cell) of 2:1 and 1:1 were used.

FIGS. 10A-10C show PTK7 CAR T cell specificity in PTK7-KO Saos2 cells (FIG. 10A) and PTK7 overexpressing A498 cells (FIG. 10B). FIG. 10C shows the PTK7 cell surface expression in the Saos2 cells, PTK7-KO Saos2 cells, A498 cells, and PTK7 overexpressing A498 cells.

FIGS. 11A-11C show that in vitro potency of PTK7 CAR T cells trended with expression pattern in solid tumor cell lines: breast cancer (FIG. 11A), pancreatic cancer (FIG. 11B), and lung (NSCLC) cancer (FIG. 11C).

FIG. 12 includes a graph showing cell proliferation of TRAC⁻/β2M⁻/anti-PTK7 CAR⁺ T cells following gene editing, compared to controls.

FIGS. 13A-13B include graphs showing persistence of multiple gene editing in T cells. Editing phenotypes as measured by FACS remained consistent from Day 7 (FIG. 13A) to Day 14 (FIG. 13B) post-editing. FIGS. 13C-13D show editing phenotype of PTK7-4 CAR-T cells measured by FACS on Day 7 post-editing presented as FACS plot (FIG. 13C) and graph (FIG. 13D).

FIGS. 14A-14F include graphs showing that TRAC⁻/β2M/anti-PTK7 CAR⁺ T cells are more effective at cell killing and secrete higher levels of IFNγ than TRAC⁻/β2M⁻/anti-CD19 CAR⁺ T cells when contacted with MCF7 (FIGS. 14A-14C) and Saos-2 (FIGS. 14D-14F) cells.

FIG. 15 shows that anti-PTK7 CAR T cells were equally efficacious in vitro in human and murine cell lines.

FIG. 16 includes a graph showing that mice treated with TRAC⁻/β2M⁻/anti-PTK7 CAR⁺ T cells showed minimal body weight loss for up to 10 days following treatment/injection.

FIG. 17 shows PTK7 cell surface expression levels in human cancer cell lines.

FIGS. 18A-18C show the efficacy of anti-PTK7 CAR T cells in various in vivo xenograft models.

FIGS. 19A-19C show blinded follow-on in vivo studies to assess PTK7 CAR T cell efficacy in ovarian (FIG. 19A), colon and SCLC (FIG. 19B), and breast (FIG. 19C) cancer types.

FIG. 20 shows the effect of PTK7 CAR T cell treatment on body weight in Hs-766T pancreatic tumor xenograft mouse model.

### DETAILED DESCRIPTION

### PTK7 Cancer Antigen

In some embodiments, the T cells of the present disclosure are engineered with a chimeric antigen receptor (CAR) designed to target PTK7. Protein tyrosine kinase 7 (PTK7), also known as colon carcinoma kinase 4 (CCK4), is receptor protein tyrosine kinase that is involved in non-canonical Wnt signaling and comprises an extracellular domain. PTK7 lacks detectable catalytic tyrosine kinase activity; however, it does comprise signal transduction activity and is presumed to function in cellular adhesion. It is further thought that PTK7 is a marker for tumor progression in cancer, as it is expressed in cancer cell lines (e.g., colon and breast cancer cell lines).

Thus, in some embodiments, T cells of the present disclosure are engineered to express a CAR comprising an anti-PTK7 antibody (e.g., anti-PTK7 scFv). In some embodiments, the anti-PTK7 antibody is an anti-PTK7 scFv encoded by the sequence of any one of SEQ ID NOs: 53, 67, 74, or 81. In some embodiments, the anti-PTK7 antibody is an anti-PTK7 scFv encoded by the sequence of any one of SEQ ID NO: 113. In some embodiments, the anti-PTK7 antibody is an anti-PTK7 scFv comprising the sequence of any one of SEQ ID NOs: 54, 68, 75, or 82. In some embodiments, the anti-PTK7 antibody is an anti-PTK7 scFv comprising a VH comprising an amino acid sequence of any one of SEQ ID NO: 55, 69, 76, or 83. In some embodiments, the anti-PTK7 antibody is an anti-PTK7 scFv comprising a VL comprising an amino acid sequence of any one of SEQ ID NO: 56, 70, 77, or 84. In some embodiments, a CAR comprising an anti-PTK7 antibody is encoded by the sequence of any one of SEQ ID NOs: 49, 51, 65, 72, or 79. In some embodiments, a CAR comprising an anti-PTK7 antibody is encoded by a sequence comprising a nucleic acid that is at least 90% identical to SEQ ID NOs: 49, 51, 65, 72, 79, or 112. In some embodiments, a CAR comprising an anti-PTK7 antibody is encoded by the sequence of any one of SEQ ID NO: 112. In some embodiments, a CAR comprising an anti-PTK7 antibody comprises the sequence of any one of SEQ ID NOs: 50, 52, 66, 73, or 80. In some embodiments, a CAR comprising an anti-PTK7 antibody comprises an anti-PTK7 antibody as described in U.S. Pat. Nos. 9,102,738 or 9,409,995.

### Multi-Gene Editing

The engineered T cells of the present disclosure, in some embodiments, include more than one gene edit, for example, in more than one gene. For example, an engineered T cell may comprise a disrupted T cell receptor alpha chain constant region (TRAC) gene, a disrupted beta-2-microglobulin (β2M) gene, a disrupted programmed cell death-1 (PD-1 or PDCD1) gene, a disrupted CD70 gene, or any combination of two or more of the foregoing disrupted genes. In some embodiments, an engineered T cell comprises a disrupted TRAC gene, a disrupted β2M gene, and a disrupted CD70 gene. In some embodiments, an engineered T cell comprises a disrupted TRAC gene, a disrupted β2M gene, and a disrupted PD-1 gene. In some embodiments, an engineered T cell comprises a disrupted TRAC gene, a disrupted β2M gene, a disrupted CD70 gene and a disrupted PD-1 gene.

It should be understood that gene disruption encompasses gene modification through gene editing (e.g., using CRISPR/Cas gene editing to insert or delete one or more nucleotides). In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

Provided herein, in some embodiments, are populations of cells in which a certain percentage of the cells has been edited (e.g., β2M gene edited), resulting in a certain percentage of cells not expressing a particular gene and/or protein. In some embodiments, at least 50% (e.g., 50%, 55%, 60%, 65%, 70%, 75%, 80%, 85%, 90%, or 85%) of the cells of a gene-edited population of cells are β2M knockout cells. In some embodiments, at least 50% of the cells (e.g. T cells) of the population do not express detectable levels of β2M protein. In some embodiments, at least 55%, at least 60%, at least 65%, at least 70%, at least 75%, at least 80%, at least 85%, at least 90%, or at least 95% of the cells of a gene-edited population of cells may be β2M knockout cells.

Methods of using CRISPR-Cas gene editing technology to create a genomic deletion in a cell (e.g., to knock out a gene in a cell) are known (Bauer D E et al. Vis. Exp. 2015; 95; e52118).

### TRAC Gene Edit

In some embodiments, an engineered T cell comprises a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. In some embodiments, a disruption in the TRAC gene expression is created by knocking a chimeric antigen receptor (CAR) into the TRAC gene (e.g., using an adeno-associated viral (AAV) vector and donor template). In some embodiments, a disruption in the TRAC gene expression is created by gRNAs targeting the TRAC genomic region. In some embodiments, a genomic deletion in the TRAC gene is created by knocking a chimeric antigen receptor (CAR) into the TRAC gene (e.g., using an AAV vector and donor template). In some embodiments, a disruption in the TRAC gene expression is created by gRNAs targeting the TRAC genomic region and knocking a chimeric antigen receptor (CAR) into the TRAC gene.

Non-limiting examples of modified and unmodified TRAC gRNA sequences that may be used as provided herein to create a genomic disruption in the TRAC gene are listed in Table 4 (e.g., SEQ ID NOs: 18 and 19). See also International Application No. PCT/US2018/032334, filed May 11, 2018, incorporated herein by reference. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region create Indels in the TRAC gene disrupting expression of the mRNA or protein.

In some embodiments, at least 50% of the engineered T cells of a population do not express a detectable level of T cell receptor (TCR) surface protein. For example, at least 55%, at least 60%, at least 70%, at least 75%, at least 80%, at least 85%, at least 90%, or at least 95% of a population may not express a detectable level of TCR surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the population of engineered T cells do not express a detectable level of TCR surface protein.

In some embodiments, gRNAs targeting the TRAC genomic region create Indels in the TRAC gene comprising at least one nucleotide sequence selected from the following sequences in Table 1:

TABLE 1

| | SEQ |
|---|---|
| Sequence | ID NO: |
| AAGAGCAACAAATCTGACT | 1 |
| AAGAGCAACAGTGCTGTGCCTGGAGCAACAAAT | 2 |
| CTGACTAAGAGCAACAAATCTGACT | |
| AAGAGCAACAGTGCTGGAGCAACAAATCTGACT | 3 |
| AAGAGCAACAAATCTGACT | |
| AAGAGCAACAGTGCCTGGAGCAACAAATCTGAC | 4 |
| TAAGAGCAACAAATCTGACT | |
| AAGAGCAACAGTGCTGACTAAGAGCAACAAATC | 5 |
| TGACT | |
| AAGAGCAACAGTGCTGTGGGCCTGGAGCAACAA | 6 |
| ATCTGACTAAGAGCAACAAATCTGACT | |
| AAGAGCAACAGTGCTGGCCTGGAGCAACAAATC | 7 |
| TGACTAAGAGCAACAAATCTGACT | |
| AAGAGCAACAGTGCTGTGTGCCTGGAGCAACAA | 8 |
| ATCTGACTAAGAGCAACAAATCTGACT | |

In some embodiments, an engineered T cell comprises a deletion in the TRAC gene relative to unmodified T cells. In some embodiments, an engineered T cell comprises a deletion of 15-30 base pairs in the TRAC gene relative to unmodified T cells. In some embodiments, an engineered T cell comprises a deletion of 15, 16, 17, 18, 19, 20, 21, 22, 23, 24, 25, 26, 27, 28, 29 or 30 base pairs in the TRAC gene relative to unmodified T cells. In some embodiments, an engineered T cell comprises a deletion of more than 30 base pairs in the TRAC gene relative to unmodified T cells. In some embodiments, an engineered T cell comprises a deletion of 20 base pairs in the TRAC gene relative to unmodified T cells. In some embodiments, an engineered T cell comprises a deletion of SEQ ID NO: 104 (AGAGCAACAGTGCTGTGGCC) in the TRAC gene relative to unmodified T cells. In some embodiments, an engineered T cell comprises a deletion comprising SEQ ID NO: 104 (AGAGCAACAGTGCTGTGGCC) in the TRAC gene relative to unmodified T cells. In some embodiments, an engineered T cell comprises a deletion of SEQ ID NO: 40 in the TRAC gene relative to unmodified T cells. In some embodiments, an engineered T cell comprises a deletion comprising SEQ ID NO: 40 in the TRAC gene relative to unmodified T cells.

### β2M Gene Edit

In some embodiments, an engineered T cell comprises a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

Non-limiting examples of modified and unmodified β2M gRNA sequences that may be used as provided herein to create a genomic disruption in the β2M gene are listed in Table 4 (e.g., SEQ ID NOs: 20 and 21). See also International Application No. PCT/US2018/032334, filed May 11, 2018, incorporated herein by reference. Other gRNA sequences may be designed using the β2M gene sequence located on Chromosome 15 (GRCh38 coordinates: Chromosome 15: 44,711,477-44,718,877; Ensembl: ENSG00000166710).

In some embodiments, gRNAs targeting the β2M genomic region create Indels in the β2M gene disrupting expression of the mRNA or protein.

In some embodiments, at least 50% of the engineered T cells of a population do not express a detectable level of β2M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population do not express a detectable level of β2M surface protein.

In some embodiments, an edited β2M gene comprises at least one nucleotide sequence selected from the following sequences in Table 2.

TABLE 2

| | SEQ |
|---|---|
| Sequences | ID NO: |
| CGTGGCCTTAGCTGTGCTCGCGCTACTCTCTCTTTCTG | 9 |
| CCTGGAGGCTATCCAGCGTGAGTCTCTCCTACCCTCCC | |
| GCT | |
| CGTGGCCTTAGCTGTGCTCGCGCTACTCTCTCTTTCGC | 10 |
| CTGGAGGCTATCCAGCGTGAGTCTCTCCTACCCTCCCG | |
| CT | |
| CGTGGCCTTAGCTGTGCTCGCGCTACTCTCTCTTTCTG | 11 |
| GAGGCTATCCAGCGTGAGTCTCTCCTACCCTCCCGCT | |
| CGTGGCCTTAGCTGTGCTCGCGCTACTCTCTCTTTCTG | 12 |
| GATAGCCTGGAGGCTATCCAGCGTGAGTCTCTCCTACC | |
| CTCCCGCT | |
| CGTGGCCTTAGCTGTGCTCGCGCTATCCAGCGTGAGTC | 13 |
| TCTCCTACCCTCCCGCT | |
| CGTGGCCTTAGCTGTGCTCGCGCTACTCTCTCTTTCTG | 14 |
| TGGCCTGGAGGCTATCCAGCGTGAGTCTCTCCTACCCT | |
| CCCGCT | |

### PD-1 Gene Edit

PD-1 is an immune checkpoint molecule that is upregulated in activated T cells and serves to dampen or stop T cell responses. Disrupting PD-1 by gene editing could lead to more persistent and/or potent therapeutic T cell responses and/or reduce immune suppression in a subject. In some embodiments, an engineered T cell comprises a disrupted PD-1 gene. In some embodiments, expression of the endogenous PD-1 gene is eliminated to enhance anti-tumor efficacy of the CAR T cells of the present disclosure.

Non-limiting examples of modified and unmodified PD-1 gRNA sequences that may be used as provided herein to create a genomic deletion in the PD-1 gene are listed in Table 4 (e.g., SEQ ID NOs: 22 and 23). See also International Application No. PCT/US2018/032334, filed May 11, 2018, incorporated herein by reference. Other gRNA sequences may be designed using the PD-1 gene sequence located on Chromosome 2 (GRCh38 coordinates: Chromosome 2: 241,849,881-241,858,908; Ensembl: ENSG00000188389).

In some embodiments, gRNAs targeting the PD-1 genomic region create Indels in the PD-1 gene disrupting expression of the PD-1 mRNA or protein.

In some embodiments, at least 50% of the engineered T cells of a population do not express a detectable level of PD-1 surface protein. For example, at least 55%, at least 60%, at least 70%, at least 75%, at least 80%, at least 85%, at least 90%, or at least 95% of the engineered T cells of a population may not express a detectable level of PD-1 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population do not express a detectable level of PD-1 surface protein.

### CD70 Gene Edit

Cluster of Differentiation 70 (CD70) is a member of the tumor necrosis factor superfamily and its expression is restricted to activated T and B lymphocytes and mature dendritic cells. CD70 has also been detected on hematological tumors and on carcinomas. CD70 is implicated in tumor cell and regulatory T cell survival through interaction with its ligand, CD27. Disrupting CD70 by gene editing increases cell expansion and reduces cell exhaustion. In some embodiments, an engineered T cell comprises a disrupted CD70 gene. In some embodiments, expression of the endogenous CD70 gene is eliminated to enhance anti-tumor efficacy of the CAR T cells of the present disclosure. In some embodiments, gRNAs targeting the CD70 genomic region create Indels in, or around, the CD70 gene disrupting expression of the CD70 mRNA and/or protein.

Non-limiting examples of modified and unmodified CD70 gRNA sequences that may be used as provided herein to create a genomic disruption in the CD70 gene are listed in Table 4 (e.g., SEQ ID NOs: 24-27). Other gRNA sequences may be designed using the CD70 gene sequence located on Chromosome 19 (GRCh38 coordinates: Chromosome 19: 6,583,183-6,604,103; Ensembl: ENSG00000125726).

In some embodiments, at least 50% of the engineered T cells of a population do not express a detectable level of CD70 surface protein. For example, at least 55%, at least 60%, at least 70%, at least 75%, at least 80%, at least 85%, at least 90%, or at least 95% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population do not express a detectable level of CD70 surface protein.

### Cellular Phenotypes

In some embodiments, one or more gene edits within a population of cells results in a phenotype associated with changes in cellular proliferative capacity, cellular exhaustion, cellular viability, cellular lysis capability (e.g., increase cytokine production and/or release), or any combination thereof.

In some embodiments, engineered T cells of the present disclosure exhibit at least 20% greater cellular proliferative capacity, relative to control T cells. For example, engineered T cells may exhibit at least 25%, at least 30%, at least 35%, at least 40%, at least 45%, at least 50%, at least 55%, at least 60%, at least 65%, at least 70%, at least 75%, at least 80%, or at least 90% greater cellular proliferative capacity, relative to control T cells. In some embodiments, engineered T cells of the present disclosure exhibit 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% greater cellular proliferative capacity, relative to control T cells.

In some embodiments, engineered T cells of the present disclosure exhibit an at least 20% increase in cellular viability, relative to control cells. For example, engineered T cells of the present disclosure may exhibit at least 25%, at least 30%, at least 35%, at least 40%, at least 45%, at least 50%, at least 55%, at least 60%, at least 65%, at least 70%, at least 75%, at least 80%, or at least 90% increase in cellular viability, relative to control cells. In some embodiments, engineered T cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular viability, relative to control cells.

In some embodiments, engineered T cells of the present disclosure exhibit an at least 20% increase in cellular lysis capability (kill at least 20% more target cells), relative to control cells. For example, engineered T cells of the present disclosure may exhibit an at least at least 25%, at least 30%, at least 35%, at least 40%, at least 45%, at least 50%, at least 55%, at least 60%, at least 65%, at least 70%, at least 75%, at least 80%, or at least 90% increase in cellular lysis capability, relative to control cells. In some embodiments, engineered T cells of the present disclosure exhibit a 20%-100%, 20%-90%, 20%-80%, 20%-70%, 20%-60%, 20%-50%, 30%-100%, 30%-90%, 30%-80%, 30%-70%, 30%-60%, 30%-50%, 40%-100%, 40%-90%, 40%-80%, 40%-70%, 40%-60%, 40%-50%, 50%-100%, 50%-90%, 50%-80%, 50%-70%, or 50%-60% increase in cellular lysis capability, relative to control cells. For example, the level of cytokines (e.g., IL-2 and/or IFN-gamma) secreted by the engineered T cells may at least 2-fold (e.g., at least 3-fold, at least 4-fold, or at least 5-fold) greater than the level of cytokines secreted by control T cells.

Control T cells, in some embodiments, are engineered T cells (e.g., gene edited T cells). In some embodiments, control T cells are engineered T cells that comprise a disrupted TRAC gene, a nucleic acid encoding a CAR (e.g., an anti-PTK7 CAR) inserted into the TRAC gene, and/or a disrupted β2M gene. In some embodiments, control T cells are unedited T cells.

### Gene Editing Methods

Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When an sequence of an endogenous gene is edited, for example by deletion, insertion or substitution of nucleotide(s)/nucleic acid(s), the endogenous gene comprising the affected sequence may be knocked-out or knocked-down due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wβ/SPBc/TP901-1, whether used individually or in combination.

Other non-limiting examples of targeted nucleases include naturally-occurring and recombinant nucleases, e.g., CRISPR/Cas9, restriction endonucleases, meganucleases homing endonucleases, and the like.

### CRISPR-Cas9 Gene Editing

The CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA)—to target the cleavage of DNA.

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

In some embodiments, the Cas9 (CRISPR associated protein 9) endonuclease is from Streptococcus pyogenes, although other Cas9 homologs may be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

### Guide RNAs

The present disclosure provides a genome-targeting nucleic acid that can direct the activities of an associated polypeptide (e.g., a site-directed polypeptide) to a specific target sequence within a target nucleic acid. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid is a double-molecule guide RNA. In some embodiments, the genome-targeting nucleic acid is a single-molecule guide RNA.

A double-molecule guide RNA comprises two strands of RNA. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (sgRNA) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins.

A single-molecule guide RNA (referred to as a “sgRNA” or “gRNA”) in a Type V system comprises, in the 5′ to 3′ direction, a minimum CRISPR repeat sequence and a spacer sequence.

The sgRNA can comprise a 20 nucleotide spacer sequence at the 5′ end of the sgRNA sequence. The sgRNA can comprise a less than 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence (see Table 3).

The sgRNA can comprise no uracil at the 3′ end of the sgRNA sequence. The sgRNA can comprise one or more uracil at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

The sgRNA can be unmodified or modified. For example, modified sgRNAs can comprise one or more 2′-O-methyl phosphorothioate nucleotides.

TABLE 3

| SEQ | |
|---|---|
| ID NO. | sgRNA sequence |
| 15 | nnnnnnnnnnnnnnnnnnnnguuuuagagcuagaaaua |
| | gcaaguuaaaauaaggcuaguccguuaucaacuugaaa |
| | aaguggcaccgagucggugcuuuu |
| 16 | nnnnnnnnnnnnnnnnnnnnguuuuagagcuagaaaua |
| | gcaaguuaaaauaaggcuaguccguuaucaacuugaaa |
| | aaguggcaccgagucggugc |
| 17 | n₍₁₇₋₃₀₎guuuuagagcuagaaauagcaaguuaaaauaa |
| | ggcuaguccguuaucaacuugaaaaaguggcaccgagu |
| | cggugcu₍₁₋₈₎ |

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacer Sequence

A gRNA comprises a spacer sequence. A spacer sequence is a sequence (e.g., a 20 nucleotide sequence) that defines the target sequence (e.g., a DNA target sequences, such as a genomic target sequence) of a target nucleic acid of interest. In some embodiments, the spacer sequence is 15 to 30 nucleotides. In some embodiments, the spacer sequence is 15, 16, 17, 18, 19, 29, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by an RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. For example, if the target sequence is 5′-AGAGCAACAGTGCTGTGGCC-3′ (SEQ ID NO: 104), then the gRNA spacer sequence is 5′-AGAGCAACAGUGCUGUGGCC-3′ (SEQ ID NO: 105). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence comprises 20 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20 bases immediately 5′ of the first nucleotide of the PAM. For example, in a sequence comprising 5′-NNNNNNNNNNNNNNNNNNNNNRG-3′, the target nucleic acid comprises the sequence that corresponds to the Ns, wherein N is any nucleotide, and the underlined NRG sequence is the S. pyogenes PAM.

Non-limiting examples of gRNAs that may be used as provided herein are provided in Table 4 and PCT/US2018/032334, filed May 11, 2018.

TABLE 4
gRNA Sequences/Target Sequences
gRNA Sequences

| | Unmodified | Modified |
|---|---|---|
| Name | Sequence | Sequence |
| TRAC sgRNA | AGAGCAACAGUGCUG | A*G*A*GCAACAGUG |
| | UGGCCguuuuagagc | CUGUGGCCguuuuag |
| | uagaaauagcaaguu | agcuagaaauagcaa |
| | aaaauaaggcuaguc | guuaaaauaaggcua |
| | cguuaucaacuugaa | guccguuaucaacuu |
| | aaaguggcaccgagu | gaaaaaguggcaccg |
| | cggugcUUUU | agucggugcU*U*U* |
| | (SEQ ID NO: 18) | U |
| | | (SEQ ID NO: 28) |
| TRAC sgRNA | AGAGCAACAGUGCUG | A*G*A*GCAACAGUG |
| spacer | UGGCC | CUGUGGCC |
| | (SEQ ID NO: 19) | (SEQ ID NO: 29) |
| β2M sgRNA | GCUACUCUCUCUUUC | G*C*U*ACUCUCUCU |
| | UGGCCguuuuagagc | UUCUGGCCguuuuag |
| | uagaaauagcaaguu | agcuagaaauagcaa |
| | aaaauaaggcuaguc | guuaaaauaaggcua |
| | cguuaucaacuugaa | guccguuaucaacuu |
| | aaaguggcaccgagu | gaaaaaguggcaccg |
| | cggugcUUUU | agucggugcU*U*U* |
| | (SEQ ID NO: 20) | U |
| | | (SEQ ID NO: 30) |
| β2M sgRNA | GCUACUCUCUCUUUC | G*C*U*ACUCUCUCU |
| spacer | UGGCC | UUCUGGCC |
| | (SEQ ID NO: 21) | (SEQ ID NO: 31) |
| PD-1 sgRNA | CUGCAGCUUCUCCAA | C*U*G*CAGCUUCUC |
| | CACAUguuuuagagc | CAACACAUguuuuag |
| | uagaaauagcaaguu | agcuagaaauagcaa |
| | aaaauaaggcuaguc | guuaaaauaaggcua |
| | cguuaucaacuugaa | guccguuaucaacuu |
| | aaaguggcaccgagu | gaaaaaguggcaccg |
| | cggugcUUUU | agucggugcU*U*U* |
| | (SEQ ID NO: 22) | U |
| | | (SEQ ID NO: 32) |
| PD-1 sgRNA | CUGCAGCUUCUCCAA | C*U*G*CAGCUUCUC |
| spacer | CACAU | CAACACAU |
| | (SEQ ID NO: 23) | (SEQ ID NO: 33) |
| CD70 sgRNA | GCUUUGGUCCCAUUG | G*C*U*UUGGUCCCA |
| (E1_T7) | GUCGCguuuuagagc | UUGGUCGCguuuuag |
| | uagaaauagcaaguu | agcuagaaauagcaa |
| | aaaauaaggcuaguc | guuaaaauaaggcua |
| | cguuaucaacuugaa | guccguuaucaacuu |
| | aaaguggcaccgagu | gaaaaaguggcaccg |
| | cggugcUUUU | agucggugcU*U*U* |
| | (SEQ ID NO: 24) | U |
| | | (SEQ ID NO: 34), |
| | | T7 |
| CD70 sgRNA | GCUUUGGUCCCAUUG | G*C*U*UUGGUCCCA |
| (E1_T7) | GUCGC | UUGGUCGC |
| spacer | (SEQ ID NO: 25) | (SEQ ID NO: 35) |
| CD70 sgRNA | GCCCGCAGGACGCAC | G*C*C*CGCAGGACG |
| (E1_T8) | CCAUAguuuuagagc | CACCCAUAguuuuag |
| | uagaaauagcaaguu | agcuagaaauagcaa |
| | aaaauaaggcuaguc | guuaaaauaaggcua |
| | cguuaucaacuugaa | guccguuaucaacuu |
| | aaaguggcaccgagu | gaaaaaguggcaccg |
| | cggugcUUUU | agucggugcU*U*U* |
| | (SEQ ID NO: 26) | U |
| | | (SEQ ID NO: 36), |
| | | T8 |
| CD70 sgRNA | GCCCGCAGGACGCAC | G*C*C*CGCAGGACG |
| (E1_T8) | CCAUA | CACCCAUA |
| spacer | (SEQ ID NO: 27) | (SEQ ID NO: 37) |

Target Sequences

| Guide Name | Target Sequence (PAM) |
|---|---|
| CD70 sgRNA | GCTTTGGTCCCATTGGTCGC |
| (E1_T7) | (GGG) (SEQ ID NO: 38) |
| CD70 sgRNA | GCCCGCAGGACGCACCCATA |
| (E1_T8) | (GGG) (SEQ ID NO: 39) |
| TRAC sgRNA | AGAGCAACAGTGCTGTGGCC |
| | (TGG) (SEQ ID NO: 40) |
| β2M sgRNA | GCTACTCTCTCTTTCTGGCC |
| | (TGG) (SEQ ID NO: 41) |
| PD-1 sgRNA | CTGCAGCTTCTCCAACACAT |
| | (CGG) (SEQ ID NO: 42) |

*: 2′-O-methyl phosphorothioate residue

### Chimeric Antigen Receptor (CAR) T Cells

A chimeric antigen receptor refers to an artificial immune cell receptor that is engineered to recognize and bind to an antigen expressed by tumor cells. Generally, a CAR is designed for a T cell and is a chimera of a signaling domain of the T-cell receptor (TCR) complex and an antigen-recognizing domain (e.g., a single chain fragment (scFv) of an antibody or other antibody fragment) (Enblad et al., Human Gene Therapy. 2015; 26(8):498-505). A T cell that expresses a CAR is referred to as a CAR T cell. CARs have the ability to redirect T-cell specificity and reactivity toward a selected target in a non-MHC-restricted manner. The non-MHC-restricted antigen recognition gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

There are four generations of CARs, each of which contains different components. First generation CARs join an antibody-derived scFv to the CD3ζeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3Z and/or co-stimulatory molecules (Maude et al., Blood. 2015; 125(26):4017-4023; Kakarla and Gottschalk, Cancer J. 2014; 20(2):151-155).

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs, but preclinical in vivo studies suggest that 4-1BB CARs may produce superior proliferation and/or persistence. Clinical trials suggest that both of these second-generation CARs are capable of inducing substantial T-cell proliferation in vivo, but CARs containing the 4-1BB costimulatory domain appear to persist longer. Third generation CARs combine multiple signaling domains (costimulatory) to augment potency.

In some embodiments, a chimeric antigen receptor is a first generation CAR. In other embodiments, a chimeric antigen receptor is a second generation CAR. In yet other embodiments, a chimeric antigen receptor is a third generation CAR.

A CAR, in some embodiments, comprises an extracellular (ecto) domain comprising an antigen binding domain (e.g., an antibody, such as an scFv), a transmembrane domain, and a cytoplasmic (endo) domain.

### Ectodomain

The ectodomain is the region of the CAR that is exposed to the extracellular fluid and, in some embodiments, includes an antigen binding domain, and optionally a signal peptide, a spacer domain, and/or a hinge domain. In some embodiments, the antigen binding domain is a single-chain variable fragment (scFv) that include the VL and VH of immunoglobulins connected with a short linker peptide. The linker, in some embodiments, includes hydrophilic residues with stretches of glycine and serine for flexibility as well as stretches of glutamate and lysine for added solubility. A single-chain variable fragment (scFv) is not actually a fragment of an antibody, but instead is a fusion protein of the variable regions of the heavy chain (VH) and light chain (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. Non-limiting examples of VH and VL protein sequences that may be used to create an anti-PTK7 scFv may include the amino acid sequence of SEQ ID NOs: 55, 69, 76, or 83 (VH) and SEQ ID NOs: 56, 70, 77, or 83 (VL). In some embodiments, the scFv of the present disclosure is humanized. In other embodiments, the scFv is fully human. In yet other embodiments, the scFv is a chimera (e.g., of mouse and human sequence). In some embodiments, the scFv is an anti-PTK7 scFv (binds specifically to PTK7). Non-limiting examples of anti-PTK7 scFv proteins that may be used as provided herein may include the amino acid sequence of any one of SEQ ID NOs: 54, 68, 75, 82. Other scFv proteins may be used.

The signal peptide can enhance the antigen specificity of CAR binding. Signal peptides can be derived from antibodies, such as, but not limited to, CD8, as well as epitope tags such as, but not limited to, GST or FLAG. Examples of signal peptides include MLLLVTSLLLCELPHPAFLLIP (SEQ ID NO: 106) and MALPVTALLLPLALLLHAARP (SEQ ID NO: 93). Other signal peptides may be used.

In some embodiments, a spacer domain or hinge domain is located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

### Transmembrane Domain

The transmembrane domain is a hydrophobic alpha helix that spans the membrane. The transmembrane domain provides stability of the CAR. In some embodiments, the transmembrane domain of a CAR as provided herein is a CD8 transmembrane domain. In other embodiments, the transmembrane domain is a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain:

(SEQ ID NO: 107)
FVPVFLPAKPTTTPAPRPPTPAPTIASQPLSLRPEACRPAAGG
AVHTRGLDFACDIYIWAPLAGTCGVLLLSLVITLYCNHRNR.

Other transmembrane domains may be used.

### Endodomain

The endodomain is the functional end of the receptor. Following antigen recognition, receptors cluster and a signal is transmitted to the cell. The most commonly used endodomain component is CD3-zeta, which contains three (3) immunoreceptor tyrosine-based activation motif (ITAM)s. This transmits an activation signal to the T cell after the antigen is bound. In many cases, CD3-zeta may not provide a fully competent activation signal and, thus, a co-stimulatory signaling is used. For example, CD28 and/or 4-1BB may be used with CD3-zeta (CD3ζ) to transmit a proliferative/survival signal. Thus, in some embodiments, the co-stimulatory molecule of a CAR as provided herein is a CD28 co-stimulatory molecule. In other embodiments, the co-stimulatory molecule is a 4-1BB co-stimulatory molecule. In some embodiments, a CAR includes CD3ζ and CD28. In other embodiments, a CAR includes CD3-zeta and 4-1BB. In still other embodiments, a CAR includes CD3ζ, CD28, and 4-1BB. Table 5 provides examples of signaling molecules that may be used as provided herein.

TABLE 5

| | | SEQ |
|---|---|---|
| Name | Sequence | ID NO: |
| 4-1BB | AAACGGGGCAGAAAGAAACTCCTGTATATATT | 43 |
| | CAAACAACCATTTATGAGACCAGTACAAACTA | |
| | CTCAAGAGGAAGATGGCTGTAGCTGCCGATTT | |
| | CCAGAAGAAGAAGAAGGAGGATGTGAACTG | |
| | KRGRKKLLYIFKQPFMRPVQTTQEEDGCSCRF | 44 |
| | PEEEEGGCEL | |
| CD28 | TCAAAGCGGAGTAGGTTGTTGCATTCCGATTA | 45 |
| | CATGAATATGACTCCTCGCCGGCCTGGGCCGA | |
| | CAAGAAAACATTACCAACCCTATGCCCCCCCA | |
| | CGAGACTTCGCTGCGTACAGGTCC | |
| | SKRSRLLHSDYMNMTPRRPGPTRKHYQPYAPP | 46 |
| | RDFAAYRS | |
| CD3- | CGAGTGAAGTTTTCCCGAAGCGCAGACGCTCC | 47 |
| zeta | GGCATATCAGCAAGGACAGAATCAGCTGTATA | |
| | ACGAACTGAATTTGGGACGCCGCGAGGAGTAT | |
| | GACGTGCTTGATAAACGCCGGGGGAGAGACCC | |
| | GGAAATGGGGGGTAAACCCCGAAGAAAGAATC | |
| | CCCAAGAAGGACTCTACAATGAACTCCAGAAG | |
| | GATAAGATGGCGGAGGCCTACTCAGAAATAGG | |
| | TATGAAGGGCGAACGACGACGGGGAAAAGGTC | |
| | ACGATGGCCTCTACCAAGGGTTGAGTACGGCA | |
| | ACCAAAGATACGTACGATGCACTGCATATGCA | |
| | GGCCCTGCCTCCCAGA | |
| | RVKFSRSADAPAYQQGQNQLYNELNLGRREEY | 48 |
| | DVLDKRRGRDPEMGGKPRRKNPQEGLYNELQK | |
| | DKMAEAYSEIGMKGERRRGKGHDGLYQGLSTA | |
| | TKDTYDALHMQALPPR | |

### Antibodies

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv), single chain variable fragment (scFv), mutants thereof, fusion proteins comprising an antibody portion, humanized antibodies, chimeric antibodies, diabodies, linear antibodies, single chain antibodies, single domain antibodies (e.g., camel or llama VHH antibodies), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

A typical antibody molecule comprises a heavy chain variable region (VH) and a light chain variable region (VL), which are usually involved in antigen binding. These regions/residues that are responsible for antigen-binding can be identified from amino acid sequences of the VH/VL sequences of a reference antibody (e.g., an anti-PTK7 antibody as described herein) by methods known in the art. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. As used herein, a CDR may refer to the CDR defined by any method known in the art. Two antibodies having the same CDR means that the two antibodies have the same amino acid sequence of that CDR as determined by the same method. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

In some embodiments, an antibody is an scFv, such as an anti-PTK7 scFv. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used as provided herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC).

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In some embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen, such as human PTK7. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a PTK7 epitope is an antibody that binds this PTK7 epitope with greater affinity, avidity, more readily, and/or with greater duration than it binds to other PTK7 epitopes or non-PTK7 epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In some embodiments, the equilibrium dissociation constant (K_{D}) between the antibody and PTK7 is 100 pM to 1 μM. In some embodiments, the K_{D} between the antibody and PTK7 is 1 nM to 100 nM.

Also within the scope of the present disclosure are functional variants of any of the exemplary antibodies as disclosed herein. A functional variant may contain one or more amino acid residue variations in the VH and/or VL, or in one or more of the VH CDRs and/or one or more of the VL CDRs as relative to a reference antibody, while retaining substantially similar binding and biological activities (e.g., substantially similar binding affinity, binding specificity, inhibitory activity, anti-tumor activity, or a combination thereof) as the reference antibody.

In some examples, an antibody disclosed herein comprises a VH CDR1, a VH CDR2, and a VH CDR3, which collectively contains no more than 10 amino acid variations (e.g., no more than 9, 8, 7, 6, 5, 4, 3, 2, or 1 amino acid variation) as compared with the VH CDR1, VH CDR2, and VH CDR3 of a reference antibody such as Antibody A (VH: SEQ ID NO: 55; VL: SEQ ID NO: 56). “Collectively” means that the total number of amino acid variations in all of the three VH CDRs is within the defined range. Alternatively or in addition, antibody may comprise a VL CDR1, a VL CDR2, and a VL CDR3, which collectively contains no more than 10 amino acid variations (e.g., no more than 9, 8, 7, 6, 5, 4, 3, 2 or 1 amino acid variation) as compared with the VL CDR1, VL CDR2, and VL CDR3 of the reference antibody.

In some examples, an antibody disclosed herein may comprise a VH CDR1, a VH CDR2, and a VH CDR3, at least one of which contains no more than 5 amino acid variations (e.g., no more than 4, 3, 2, or 1 amino acid variation) as the counterpart VH CDR of a reference antibody such as Antibody A (VH: SEQ ID NO: 55; VL: SEQ ID NO: 56). In specific examples, the antibody comprises a VH CDR3, which contains no more than 5 amino acid variations (e.g., no more than 4, 3, 2, or 1 amino acid variation) as the VH CDR3 of a reference antibody such as Antibody A (VH: SEQ ID NO: 55; VL: SEQ ID NO: 56). Alternatively or in addition, an antibody may comprise a VL CDR1, a VL CDR2, and a VL CDR3, at least one of which contains no more than 5 amino acid variations (e.g., no more than 4, 3, 2, or 1 amino acid variation) as the counterpart VL CDR of the reference antibody. In specific examples, the antibody comprises a VL CDR3, which contains no more than 5 amino acid variations (e.g., no more than 4, 3, 2, or 1 amino acid variation) as the VL CDR3 of the reference antibody.

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (I) K→R, H; (m) M→L, I, Y; (n) F→Y, M, L; (o) P→A; (p) S→T; (q) T→S; (r) W→Y, F; (s) Y→W, F; and (t) V→I, L.

In some embodiments, an antibody disclosed herein may comprise VH CDRs that collectively are at least 80% (e.g., 85%, 90%, 95%, or 98%) identical to the VH CDRs of a reference antibody such as Antibody A (VH: SEQ ID NO: 55; VL: SEQ ID NO: 56). Alternatively or in addition, the antibody may comprise VL CDRs that collectively are at least 80% (e.g., 85%, 90%, 95%, or 98%) identical to the VL CDRs of the reference antibody. In some embodiments, an antibody may comprise a VH that is at least 80% (e.g., 85%, 90%, 95%, or 98%) identical to the VH of a reference antibody such as Antibody A (VH: SEQ ID NO: 55; VL: SEQ ID NO: 56) and/or a VL that is at least 80% (e.g., 85%, 90%, 95%, or 98%) identical to the VL of the reference antibody.

### Donor Template

The nucleic acid encoding a CAR may be delivered to a T cell that comprises what is referred to herein as a donor template (also referred to as a donor polynucleotide). A donor template can contain a non-homologous sequence, such as the nucleic acid encoding a CAR, flanked by two regions of homology to allow for efficient HDR at a genomic location of interest. In some embodiments, the region of homology can comprise a nucleotide sequence of SEQ ID NO: 92 or 100. In some embodiments, the non-homologous sequence is flanked by a nucleotide sequence of SEQ ID NO: 92 and a nucleotide sequence of SEQ ID NO: 100. Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter at the integration site, namely the promoter that drives expression of the endogenous gene into which the donor is inserted. However, in some embodiments, the donor template comprises an exogenous promoter and/or enhancer, for example a constitutive promoter, an inducible promoter, or tissue-specific promoter. In some embodiments, the exogenous promoter is an EF1α promoter comprising a sequence of SEQ ID NO: 101. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, the donor template comprises the nucleotide sequence of any one of SEQ ID NOs: 63, 64, 71, 78, or 91.

### Delivery Methods and Constructs

Nucleases and/or donor templates may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron® 2000 system (Rich-Mar) can also be used for delivery of nucleic acids.

### Adeno-Associated Viral Delivery

The donor nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

### Homology-Directed Repair (HDR)

The donor nucleic acid encoding a CAR is inserted by homology directed repair (HDR) into the target gene locus. Both strands of the DNA at the target locus are cut by a CRISPR Cas9 enzyme. HDR then occurs to repair the double-strand break (DSB) and insert the donor DNA. For this to occur correctly, the donor sequence is designed with flanking residues which are complementary to the sequence surrounding the DSB site in the target gene (hereinafter “homology arms”). These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

The target gene can be associated with an immune response in a subject, wherein permanently deleting at least a portion of the target gene will modulate the immune response. For example, to generate a CAR T cell, the target gene can be the TCRα constant region (TRAC). Disruption of TRAC leads to loss of function of the endogenous TCR.

In some embodiments, the target gene is in a safe harbor locus.

### Engineered T Cells

Engineered (gene edited) CAR T cells of the present disclosure may be autologous (“self”) or non-autologous (“non-self,” e.g., allogeneic, syngeneic or xenogeneic). “Autologous” refers to cells from the same subject. “Allogeneic” refers to cells of the same species as a subject, but that differ genetically to the cells in the subject. In some embodiments, the T cells are obtained from a mammalian subject. In some embodiments, the T cells are obtained from a human subject.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some embodiments, an isolated population of T cells is used. In some embodiments, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, the engineered T cell populations do not express or do not substantially express one or more of the following markers: CD70, CD57, CD244, CD160, PD-1, CTLA4, HM3, and LAG3. In some embodiments, subpopulations of T cells may be isolated by positive or negative selection prior to genetic engineering and/or post genetic engineering.

In some embodiments, an isolated population of T cells expresses one or more of the markers including, but not limited to a CD3+, CD4+, CD8+, or a combination thereof. In some embodiments, the T cells are isolated from a subject and first activated and stimulated to proliferate in vitro prior to undergoing gene editing.

To achieve sufficient therapeutic doses of T cell compositions, T cells are often subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells.

In some embodiments, T cells are activated at the same time that genome editing compositions are introduced into the T cells.

Also provided are populations of engineered T cells described herein. In some embodiments, at least 25% to 100% of the engineered T cells of the population express the CAR. In some embodiments, at least 25% or at least 50% of the engineered T cells of the population express the CAR. In some embodiments, at least 70% of the engineered T cells of the population express the CAR. In some embodiments, at least 25% of engineered T cells of the population express the CAR following at least 7 or at least 14 days of in vitro proliferation.

### Treatment Methods and Compositions

Provided herein, in some embodiments, are methods for treating cancer (e.g.: solid tumors). Non-limiting examples of solid tumors that may be treated as provided herein include: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas and/or melanoma. In some embodiments, the cancer is selected from the group consisting of: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, and intrahepatic biliary cancer. In some embodiments, the methods comprise delivering the CAR T cells (e.g., anti-Ptk7 CAR T cells) of the present disclosure to a subject having cancer (e.g., solid tumors) including, pancreatic cancer, gastric cancer, ovarian cancer, cervical cancer, breast cancer, prostate cancer, testicular cancer, thyroid cancer, nasopharyngeal cancer, non-small cell lung (NSCLC), glioblastoma, and/or melanoma. In some embodiments, the methods comprise delivering the CAR T cells (e.g., anti-PTK7 CAR T cells) of the present disclosure to a subject having a leukemia or a lymphoma, e.g., leukemia or lymphomas of T cells, B cell, NK cell, dendritic cells. Non-limiting examples of leukemias include acute lymphoblastic leukemia (ALL), acute myeloid leukemia (AML), chronic lymphocytic leukemia (CLL) and chronic myeloid leukemia (CML).

The step of administering may include the placement (e.g., transplantation) of cells, e.g., engineered T cells, into a subject, by a method or route that results in at least partial localization of the introduced cells at a desired site, such as tumor, such that a desired effect(s) is produced. Engineered T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of engineered T cells is administered via a systemic route of administration, such as an intraperitoneal or intravenous route.

A subject may be any subject for whom diagnosis, treatment, or therapy is desired. In some embodiments, the subject is a mammal. In some embodiments, the subject is a human.

A donor is an individual who is not the subject being treated. A donor is an individual who is not the patient. In some embodiments, a donor is an individual who does not have or is not suspected of having the cancer being treated. In some embodiments, multiple donors, e.g., two or more donors, are used.

In some embodiments, an engineered T cell population being administered according to the methods described herein comprises allogeneic T cells obtained from one or more donors. Allogeneic refers to a cell, cell population, or biological samples comprising cells, obtained from one or more different donors of the same species, where the genes at one or more loci are not identical to the recipient. For example, an engineered T cell population, being administered to a subject can be derived from one or more unrelated donors, or from one or more non-identical siblings. In some embodiments, syngeneic cell populations may be used, such as those obtained from genetically identical donors, (e.g., identical twins). In some embodiments, the cells are autologous cells; that is, the engineered T cells are obtained or isolated from a subject and administered to the same subject, i.e., the donor and recipient are the same.

In some embodiments, an engineered T cell population being administered according to the methods described herein does not induce toxicity in the subject, e.g., the engineered T cells do not induce toxicity in non-cancer cells. In some embodiments, an engineered T cell population being administered does not trigger complement mediated lysis, or does not stimulate antibody-dependent cell mediated cytotoxicity (ADCC).

An effective amount refers to the amount of a population of engineered T cells needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

For use in the various aspects described herein, an effective amount of cells (e.g., engineered T cells) comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The cells are derived from one or more donors, or are obtained from an autologous source. In some examples described herein, the cells are expanded in culture prior to administration to a subject in need thereof.

Modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

In some embodiments, engineered T cells are administered systemically, which refers to the administration of a population of cells other than directly into a target site, tissue, or organ, such that it enters, instead, the subject's circulatory system and, thus, is subject to metabolism and other like processes.

The efficacy of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

The present disclosure is exemplified by the following embodiments:

Embodiment 1. An engineered T cell comprising a nucleic acid encoding a chimeric antigen receptor (CAR), wherein the CAR comprises an ectodomain that binds specifically to PTK7.

Embodiment 2. The engineered T cell of embodiment 1 further comprising a disrupted T cell receptor alpha chain constant region (TRAC) gene.

Embodiment 3. The engineered T cell of embodiment 2, wherein the nucleic acid encoding the CAR is inserted into the disrupted TRAC gene.

Embodiment 4. The engineered T cell of any one of embodiments 1-3 further comprising a disrupted beta-2-microglobulin (β2M) gene.

Embodiment 5. The engineered T cell of any one of embodiments 1-4, wherein the ectodomain of the CAR comprises an anti-PTK7 antibody.

Embodiment 6. The engineered T cell of embodiment 5, wherein the anti-PTK7 antibody is an anti-PTK7 single-chain variable fragment (scFv).

Embodiment 7. The engineered T cell of embodiment 6, wherein the anti-PTK7 scFv comprises the same heavy chain variable domain (VH) complementarity determining regions (CDRs) and the same light chain variable domain (VL) CDRs as a reference antibody, wherein the reference antibody comprises (i) a VH set forth as SEQ ID NO: 55 and a VL set forth as SEQ ID NO: 56, (ii) VH set forth as SEQ ID NO: 69 and a VL set forth as SEQ ID NO: 70, (iii) a VH set forth as SEQ ID NO: 76 and a VL set forth as SEQ ID NO: 77, or (iv) a VH set forth as SEQ ID NO: 83 and a VL set forth as SEQ ID NO: 84.

Embodiment 8. The engineered T cell of embodiment 7, wherein the anti-PTK7 scFv comprises the same VH and VL chains as the reference antibody.

Embodiment 9. The engineered T cell of embodiment 7, wherein the anti-PTK7 scFv comprises the amino acid sequence of any one of SEQ ID NOs: 54, 68, 75, or 82.

Embodiment 10. The engineered T cell of any one of embodiments 1-9, wherein the CAR comprises a CD28 co-stimulatory domain or a 41BB co-stimulatory domain.

Embodiment 11. The engineered T cell of embodiment 10, wherein the CAR further comprises a CD3ζ cytoplasmic signaling domain.

Embodiment 12. The engineered T cell of any one of embodiments 3-11, wherein the TRAC gene comprises the nucleotide sequence encoding the LHA and/or RHA within any one of SEQ ID NOs: 63, 64, 71, 78, or 91 or the nucleotide sequence of SEQ ID NO: 92 or 100, and/or wherein the CAR is encoded by the nucleotide sequence of any one of SEQ ID NOs: 49, 51, 65, 72, 79, or 112.

Embodiment 13. The engineered T cell of any one of embodiments 4-12, wherein the disrupted β2M gene comprises at least one nucleotide sequence selected from any one of SEQ ID NOs: 9-14.

Embodiment 14. A population of the engineered T cell of any one of embodiments 1-13, wherein at least 25% or at least 50% of engineered T cells of the population express the CAR.

Embodiment 15. The population of embodiment 14, wherein at least 70% of engineered T cells of the population express the CAR.

Embodiment 16. The population of embodiments 14 or 15, wherein at least 25% of engineered T cells of the population express the CAR following at least 7 or at least 14 days of in vitro proliferation.

Embodiment 17. The population of any one of embodiments 14-16, wherein at least 50% of engineered T cells of the population do not express a detectable level of T cell receptor (TCR) protein.

Embodiment 18. The population of embodiment 17, wherein at least 90% of engineered T cells of the population do not express a detectable level of TCR protein.

Embodiment 19. The population of any one of embodiments 14-18, wherein at least 50% of engineered T cells of the population do not express a detectable level of β2M protein.

Embodiment 20. The population of embodiment 19, wherein at least 70% of engineered T cells of the population do not express a detectable level of β2M protein.

Embodiment 21. The population of any one of embodiments 14-20, wherein engineered T cells of the population, when co-cultured in vitro with a population of cancer cells that express PTK7, induce cell lysis of at least 10%, at least 25%, or at least 50% of the cancer cells of the population.

Embodiment 22. The population of embodiment 21, wherein engineered T cells of the population, when co-cultured in vitro with a population of cancer cells that express PTK7, induce cell lysis of at least 70%, at least 80%, or at least 90% of the population of cancer cells.

Embodiment 23. The population of embodiments 21 or 22, wherein engineered T cells of the population, when co-cultured in vitro with a population of cancer cells, secrete IFNγ.

Embodiment 24. The population of any one of embodiments 21-23, wherein the ratio of engineered T cells to cancer cells is 1:1 to 2:1.

Embodiment 25. The population of any one of embodiments 21-24, wherein the cancer cells comprise sarcoma cells.

Embodiment 26. The population of any one of embodiments 21-24, wherein the cancer cells comprise breast cancer cells, ovarian cancer cells, small cell lung cancer cells, and/or colon cancer cells.

Embodiment 28. The population of any one of embodiments 14-27, when administered in vivo to a subject, does not induce toxicity in the subject.

Embodiment 29. A method comprising administering the population of engineered T cells any one of embodiments 14-28 to a subject.

Embodiment 30. The method of embodiment 29, wherein the subject is a human subject.

Embodiment 31. The method of embodiment 30, wherein the subject has a cancer.

Embodiment 32. The method of embodiment 31, wherein the cancer is selected from the group consisting of: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, and intrahepatic biliary cancer.

Embodiment 33. The method of embodiments 31 or 32, wherein the cancer comprises cancer cells expressing PTK7.

Embodiment 34. A method for producing an engineered T cell, the method comprising (a) delivering to a T cell a RNA-guided nuclease, a gRNA targeting a TRAC gene, and a vector comprising a donor template that comprises a nucleic acid encoding a CAR that comprise an ectodomain that binds specifically to PTK7; and (b) producing an engineered T cell having a disrupted TRAC gene and expressing the CAR.

Embodiment 35. The method of embodiment 34, wherein the gRNA targeting the TRAC gene comprises the nucleotide sequence of SEQ ID NO: 18 or 19, or targets the nucleotide sequence of SEQ ID NO: 40.

Embodiment 36. The method of embodiments 34 or 35, wherein the nucleic acid encoding the CAR is flanked by left and right homology arms to the TRAC gene.

Embodiment 37. The method of any one of embodiments 34-36 further comprising delivering to the T cell a gRNA targeting the β2M gene.

Embodiment 38. The method of embodiment 37, wherein the gRNA targeting the β2M gene comprises the nucleotide sequence of SEQ ID NO: 20 or 21, or targets the nucleotide sequence of SEQ ID NO: 41.

Embodiment 39. The method of any one of embodiments 34-38, wherein the RNA-guided nuclease is a Cas9 nuclease, optionally a S. pyogenes Cas9 nuclease.

Embodiment 40. The method of any one of embodiments 34-39, wherein the ectodomain of the CAR is an anti-PTK7 antibody.

Embodiment 41. The method of embodiment 40, wherein the anti-PTK7 antibody is an anti-PTK7 single-chain variable fragment (scFv).

Embodiment 42. The method of embodiment 41, wherein the anti-PTK7 scFv comprises the same heavy chain variable domain (VH) complementarity determining regions (CDRs) and the same light chain variable domain (VL) CDRs as a reference antibody, wherein the reference antibody comprises (i) a VH set forth as SEQ ID NO: 55 and VL set forth as SEQ ID NO: 56, (ii) a VH set forth as SEQ ID NO: 69 and a VL set forth as SEQ ID NO: 70, (iii) a VH set forth as SEQ ID NO: 76 and a VL set forth as SEQ ID NO: 77, or (iv) a VH set forth as SEQ ID NO: 83 and a VL set forth as SEQ ID NO: 84.

Embodiment 43. The method of embodiment 42, wherein the anti-PTK7 scFv comprises the same VH and VL chains as the reference antibody.

Embodiment 44. The method of embodiment 42, wherein the anti-PTK7 scFv comprises the amino acid sequence of any one of SEQ ID NOs: 54, 68, 75, or 82.

Embodiment 45. The method of any one of embodiments 34-44, wherein the CAR comprises a CD28 co-stimulatory domain or a 41BB co-stimulatory domain.

Embodiment 46. The method of embodiment 45, wherein the CAR further comprises a CD3ζ cytoplasmic signaling domain.

Embodiment 47. The method of any one of embodiments 34-46, wherein the donor template comprises the nucleotide sequence of any one of SEQ ID NOs: 63, 64, 71, 78, or 91.

Embodiment 48. The method of any one of embodiments 34-47, wherein the CAR is encoded by a nucleotide sequence of any one of SEQ ID NOs: 49, 51, 65, 72, 79, or 112.

The present disclosure is further exemplified by the following embodiments:

Embodiment A1. An engineered T cell comprising a nucleic acid encoding a chimeric antigen receptor (CAR), wherein the CAR comprises an ectodomain that binds specifically to PTK7.

Embodiment A2. The engineered T cell of embodiment A1 further comprising a disrupted T cell receptor alpha chain constant region (TRAC) gene.

Embodiment A3. The engineered T cell of embodiment A1 or A2 further comprising a disrupted beta-2-microglobulin (β2M) gene.

Embodiment A4. The engineered T cell of any one of embodiments A1-A3, wherein the ectodomain of the CAR comprises an anti-PTK7 antibody.

Embodiment A5. The engineered T cell of embodiment A4, wherein the anti-PTK7 antibody is an anti-PTK7 single-chain variable fragment (scFv).

Embodiment A6. The engineered T cell of embodiment A5, wherein the anti-PTK7 scFv comprises the same heavy chain variable domain (VH) complementarity determining regions (CDRs) and the same light chain variable domain (VL) CDRs as a reference antibody, wherein the reference antibody comprises (i) a VH set forth as SEQ ID NO: 55 and a VL set forth as SEQ ID NO: 56, (ii) a VH set forth as SEQ ID NO: 69 and a VL set forth as SEQ ID NO: 70, (iii) a VH set forth as SEQ ID NO: 76 and a VL set forth as SEQ ID NO: 77, or (iv) a VH set forth as SEQ ID NO: 83 and a VL set forth as SEQ ID NO: 84.

Embodiment A7. The engineered T cell of embodiment A6, wherein the anti-PTK7 scFv comprises the same VH and VL chains as the reference antibody.

Embodiment A8. The engineered T cell of embodiment A6, wherein the anti-PTK7 scFv comprises the amino acid sequence of any one of SEQ ID NOs: 54, 68, 75, or 82.

Embodiment A9. The engineered T cell of any one of embodiments A1-A8, wherein the CAR further comprises a CD28 co-stimulatory domain or a 41BB co-stimulatory domain.

Embodiment A10. The engineered T cell of embodiment A9, wherein the CAR further comprises a CD3ζ cytoplasmic signaling domain.

Embodiment A11. The engineered T cell of any one of embodiments A1-A10, wherein the CAR is encoded by the nucleotide sequence of any one of SEQ ID NOs: 49, 51, 65, 72, 79, or 112 or a nucleotide sequence comprising a nucleic acid sequence that is at least 90% identical to SEQ ID NOs: 49, 51, 65, 72, 79, or 112.

Embodiment A12. The engineered T cell of any one of embodiments A1-A11, wherein the nucleic acid encoding the CAR is inserted into the disrupted TRAC gene.

Embodiment A13. The engineered T cell of any one of embodiments A2-A12, wherein the disrupted TRAC gene comprises the nucleotide sequence encoding the LHA and/or RHA within any one of SEQ ID NOs: 63, 64, 71, 78, or 91, the nucleotide sequence of SEQ ID NO: 92 or 100, and/or the nucleotide sequence of any one of SEQ ID NOs: 63, 64, 71, 78, or 91.

Embodiment A14. The engineered T cell of any one of embodiments A1-A13, wherein the disrupted β2M gene comprises at least one nucleotide sequence selected from any one of SEQ ID NOs: 9-14.

Embodiment A15. An engineered T cell comprising: (i) a disrupted TRAC gene; (ii) a disrupted β2M gene; and (iii) a nucleic acid encoding a CAR comprising an anti-PTK7 antigen-binding fragment.

Embodiment A16. The engineered T cell of embodiment A15, wherein the CAR comprises (a) an ectodomain that comprises an anti-PTK7 antigen-binding fragment, (b) a CD8 transmembrane domain, and (c) an endodomain that comprises a CD28 co-stimulatory domain and a CD3ζ cytoplasmic signaling domain.

Embodiment A17. The engineered T cell of embodiment A15 or A16, wherein the disrupted TRAC gene comprises the nucleic acid encoding the CAR.

Embodiment A18. An engineered T cell comprising: (i) a disrupted TRAC gene, wherein the disrupted TRAC gene comprises a nucleic acid encoding a CAR comprising (a) an ectodomain that comprises an anti-PTK7 antigen-binding fragment, (b) a CD8 transmembrane domain, and (c) an endodomain that comprises a CD28 co-stimulatory domain and a CD3ζ cytoplasmic signaling domain; and (ii) a disrupted β2M gene.

Embodiment A19. An engineered T cell comprising: (i) a disrupted TRAC gene, wherein the disrupted TRAC gene comprises a nucleic acid encoding a CAR comprising an amino acid sequence of any one of SEQ ID NOs: 50, 52, 66, 73, or 80; and (ii) a disrupted β2M gene.

Embodiment A20. An engineered T cell comprising: (i) a disrupted TRAC gene, wherein the disrupted TRAC gene comprises a nucleic acid encoding a CAR, wherein the nucleic acid sequence is at least 90% identical to SEQ ID NOs: 49, 51, 65, 72, 79, or 112 and encodes a CAR comprising an amino acid sequence of SEQ ID NOs: 50, 52, 66, 73, or 80; and (ii) a disrupted β2M gene.

Embodiment A21. The engineered T cell of any one of embodiments A1-A20, wherein the T cell is a human T cell.

Embodiment A22. A population of cells comprising the engineered T cell of any one of embodiments A1-A21, wherein at least 25% or at least 50% of engineered T cells of the population express the CAR.

Embodiment A23. The population of embodiment A22, wherein at least 70% of engineered T cells of the population express the CAR.

Embodiment A24. The population of embodiment A22, wherein at least 25% of engineered T cells of the population express the CAR following at least 7 days or at least 14 days of in vitro proliferation.

Embodiment A25. The population of any one of embodiments A22-A24, wherein at least 50% of engineered T cells of the population do not express a detectable level of T cell receptor (TCR) protein.

Embodiment A26. The population of embodiment A25, wherein at least 90% of engineered T cells of the population do not express a detectable level of TCR protein.

Embodiment A27. The population of any one of embodiments A22-A26, wherein at least 50% of engineered T cells of the population do not express a detectable level of β2M protein.

Embodiment A28. The population of embodiment A27, wherein at least 70% of engineered T cells of the population do not express a detectable level of β2M protein.

Embodiment A29. The population of any one of embodiments A22-A28, wherein engineered T cells of the population, when co-cultured in vitro with a population of cancer cells that express PTK7, induce cell lysis of at least 10%, at least 25%, or at least 50% of the cancer cells of the population.

Embodiment A30. The population of embodiment A29, wherein engineered T cells of the population, when co-cultured in vitro with a population of cancer cells that express PTK7, induce cell lysis of at least 70%, at least 80%, or at least 90% of the population of cancer cells.

Embodiment A31. The population of embodiment A29 or A30, wherein engineered T cells of the population, when co-cultured in vitro with a population of cancer cells, secrete IFNγ.

Embodiment A32. The population of any one of embodiments A29-A31, wherein the ratio of engineered T cells to cancer cells is 1:1 to 2:1.

Embodiment A33. The population of any one of embodiments A29-A32, wherein the cancer cells comprise sarcoma cells.

Embodiment A34. The population of any one of embodiments A29-3A2, wherein the cancer cells comprise breast cancer cells, ovarian cancer cells, small cell lung cancer cells, and/or colon cancer cells.

Embodiment A35. The population of any one of embodiments A22-A34, when administered in vivo to a subject, does not induce toxicity in the subject.

Embodiment A36. A population of cells comprising engineered T cells, wherein the engineered T cells comprise: (i) a disrupted TRAC gene; (ii) a disrupted β2M gene; and (iii) a nucleic acid encoding a CAR comprising an anti-PTK7 antigen-binding fragment.

Embodiment A37. The population of cells of embodiment A36, wherein the CAR comprises (a) an ectodomain that comprises an anti-PTK7 antigen-binding fragment, (b) a CD8 transmembrane domain, and (c) an endodomain that comprises a CD28 co-stimulatory domain and a CD3ζ cytoplasmic signaling domain.

Embodiment A38. The population of cells of embodiment A36 or A37, wherein the disrupted TRAC gene comprises the nucleic acid encoding the CAR.

Embodiment A39. A population of cells comprising engineered T cells, wherein the engineered T cells comprise: (i) a disrupted TRAC gene, wherein the disrupted TRAC gene comprises a nucleic acid encoding a CAR comprising (a) an ectodomain that comprises an anti-PTK7 antigen-binding fragment, (b) a CD8 transmembrane domain, and (c) an endodomain that comprises a CD28 co-stimulatory domain and a CD3ζ cytoplasmic signaling domain; and (ii) a disrupted β2M gene.

Embodiment A40. A population of cells comprising engineered T cells, wherein the engineered T cells comprise: (i) a disrupted TRAC gene, wherein the disrupted TRAC gene comprises a nucleic acid encoding a CAR, wherein the nucleic acid sequence is at least 90% identical to SEQ ID NOs: 49, 51, 65, 72, 79, or 112 and encodes the CAR of SEQ ID NOs: 50, 52, 66, 73, or 80; and (ii) a disrupted β2M gene.

Embodiment A41. A method comprising administering the population of engineered T cells any one of embodiments A22-A40 to a subject.

Embodiment A42. The method of embodiment A41, wherein the subject is a human subject.

Embodiment A43. The method of embodiment A42, wherein the subject has a cancer.

Embodiment A44. The method of embodiment A43, wherein the cancer is selected from the group consisting of: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, and intrahepatic biliary cancer.

Embodiment A45. The method of embodiment A43 or A44, wherein the cancer comprises cancer cells expressing PTK7.

Embodiment A46. A method for producing an engineered T cell, the method comprising (a) delivering to a T cell (i) a RNA-guided nuclease, (ii) a gRNA targeting a TRAC gene, and (iii) a vector comprising a donor template that comprises a nucleic acid encoding a CAR that comprise an ectodomain that binds specifically to PTK7; and (b) producing an engineered T cell having a disrupted TRAC gene and expressing the CAR.

Embodiment A47. The method of embodiment A46, wherein the gRNA targeting the TRAC gene comprises the nucleotide sequence of SEQ ID NO: 18 or 19, or targets the nucleotide sequence of SEQ ID NO: 40.

Embodiment A48. The method of embodiment A46 or A47 further comprising delivering to the T cell a gRNA targeting the β2M gene.

Embodiment A49. The method of embodiment A48, wherein the gRNA targeting the β2M gene comprises the nucleotide sequence of SEQ ID NO: 20 or 21, or targets the nucleotide sequence of SEQ ID NO: 41.

Embodiment A50. The method of any one of embodiments A46-A49, wherein the ectodomain of the CAR comprises an anti-PTK7 antibody.

Embodiment A51. The method of embodiment A50, wherein the anti-PTK7 antibody is an anti-PTK7 single-chain variable fragment (scFv).

Embodiment A52. The method of embodiment A51, wherein the anti-PTK7 scFv comprises the same heavy chain variable domain (VH) complementarity determining regions (CDRs) and the same light chain variable domain (VL) CDRs as a reference antibody, wherein the reference antibody comprises (i) a VH set forth as SEQ ID NO: 55 and VL set forth as SEQ ID NO: 56, (ii) a VH set forth as SEQ ID NO: 69 and a VL set forth as SEQ ID NO: 70, (iii) a VH set forth as SEQ ID NO: 76 and a VL set forth as SEQ ID NO: 77, or (iv) a VH set forth as SEQ ID NO: 83 and a VL set forth as SEQ ID NO: 84.

Embodiment A53. The method of embodiment A52, wherein the anti-PTK7 scFv comprises the same VH and VL chains as the reference antibody.

Embodiment A54. The method of embodiment A52, wherein the anti-PTK7 scFv comprises the amino acid sequence of any one of SEQ ID NOs: 54, 68, 75, or 82.

Embodiment A55. The method of any one of embodiments A46-A54, wherein the CAR further comprises a CD28 co-stimulatory domain or a 41BB co-stimulatory domain.

Embodiment A56. The method of embodiment A55, wherein the CAR further comprises a CD3ζ cytoplasmic signaling domain.

Embodiment A57. The method of any one of embodiments A46-A56, wherein the CAR is encoded by a nucleotide sequence of any one of SEQ ID NOs: 49, 51, 65, 72, 79, or 112 or a nucleotide sequence comprising a nucleic acid sequence that is at least 90% identical to SEQ ID NOs: 49, 51, 65, 72, 79, or 112.

Embodiment A58. The method of any one of embodiments A46-A57, wherein the nucleic acid encoding the CAR is flanked by left and right homology arms to the TRAC gene.

Embodiment A59. The method of any one of embodiments A46-A58, wherein the donor template comprises the nucleotide sequence of any one of SEQ ID NOs: 63, 64, 71, 78, or 91.

Embodiment A60. The method of any one of embodiments A46-A59, wherein the RNA-guided nuclease is a Cas9 nuclease, optionally a S. pyogenes Cas9 nuclease.

Embodiment A61. An engineered T cell produced by the method of any one of embodiments A46-A60.

Embodiment A62. A population of cells comprising the engineered T cell of embodiment A61.

Embodiment A63. A method of treating cancer in a subject, comprising administering to the subject the population of cells of any one of embodiments A22-A40 or A62.

Embodiment A64. The method of embodiment A63, wherein the cancer is selected from the group consisting of: pancreatic cancer, gastric cancer, ovarian cancer, uterine cancer, breast cancer, prostate cancer, testicular cancer, thyroid cancer, nasopharyngeal cancer, non-small cell lung (NSCLC), glioblastoma, neuronal, soft tissue sarcomas, leukemia, lymphoma, melanoma, colon cancer, colon adenocarcinoma, brain glioblastoma, hepatocellular carcinoma, liver hepatocholangiocarcinoma, osteosarcoma, gastric cancer, esophagus squamous cell carcinoma, advanced stage pancreas cancer, lung adenocarcinoma, lung squamous cell carcinoma, lung small cell cancer, renal carcinoma, and intrahepatic biliary cancer.

Embodiment A65. The method of embodiments A63 or A64, wherein the cancer comprises cancer cells expressing PTK7.

### EXAMPLES

### Example 1. PTK7 Expression in Normal Human Tissues

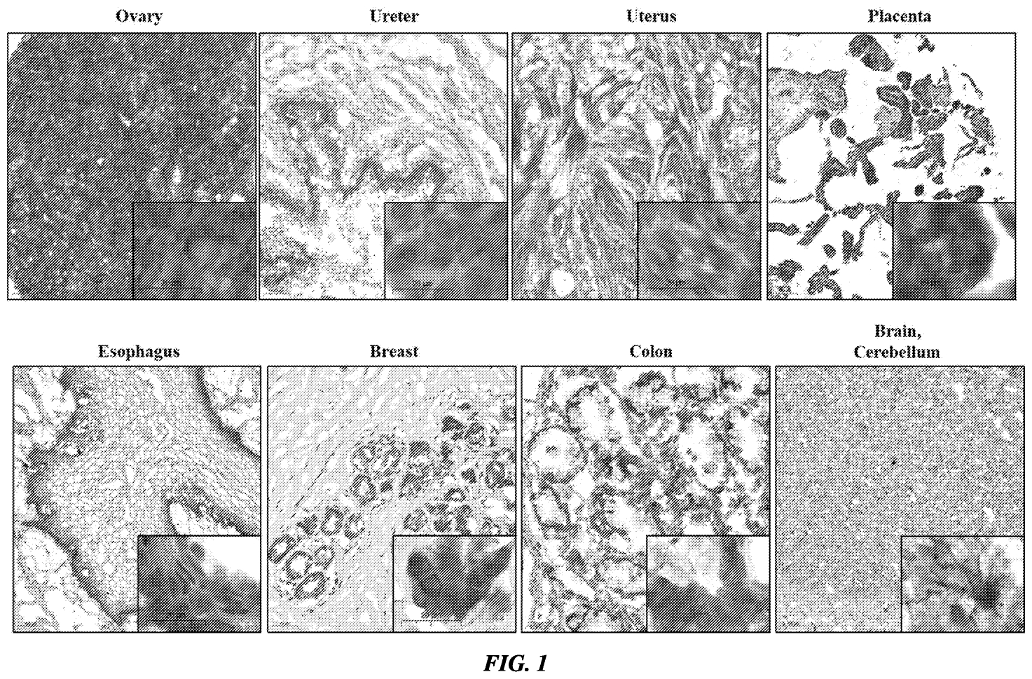

PTK7 expression was evaluated on a frozen normal human tissue panel (FDA Standard, Biochain) using a custom recombinant monoclonal biotinylated antibody (CTX181 mAb, Creative Biolabs, 1 mg/ml, SEQ ID NO: 108 and SEQ ID NO: 109) specific to the PTK7 CAR construct or a biotinylated mouse isotype control (Novus, NBP2-21948). Slides were fixed with −20° C. acetone for 10 min at ambient temperature followed by a manual immunohistochemical staining at ambient temperature. Blocking to minimize non-specific staining was performed with Peroxidased 1, Avidin and Biotin and Background Sniper (BioCare Medical, PX968M, AB972L, BS966M) in sequential steps. Slides were stained with the biotinylated CTX181 primary monoclonal antibody (1:600) for 30 min followed by incubation with 4plus Streptavidin-HRP Label (BioCare Medical) reagent for 15 min each. Visualization of target antigen was visualized with DAB (brown color) substrate chromogen (Dako, K3468). Mayer's Hematoxylin (Dako, S3309) was used to counterstain the cell nuclei. FIG. 1 shows that PTK7 expression was not widespread in normal human tissues.

Sequences for CTX181 mAb:
>181_HC
(SEQ ID NO: 108)
QVQLVESGGGVVQPGRSLRLSCAASGFTFSSYGMHWVRQAPGKGL
EWVAVIWDDGSNKYYVDSVKGRFTISRDNSKNTLYLQMNSLRAED
TAVYYCARDDYYGSGSFNSYYGTDVWGQGTTVTVSSASTKGPSVF
PLAPSSKSTSGGTAALGCLVKDYFPEPVTVSWNSGALTSGVHTFP
AVLQSSGLYSLSSVVTVPSSSLGTQTYICNVNHKPSNTKVDKKVE
PKSCDKTHTCPPCPAPELLGGPSVFLFPPKPKDTLMISRTPEVTC
VVVDVSHEDPEVKFNWYVDGVEVHNAKTKPREEQYNSTYRVVSVL
TVLHQDWLNGKEYKCKVSNKALPAPIEKTISKAKGQPREPQVYTL
PPSRDELTKNQVSLTCLVKGFYPSDIAVEWESNGQPENNYKTTPP
VLDSDGSFFLYSKLTVDKSRWQQGNVFSCSVMHEALHNHYTQKSL
SLSPGK
>181_LC
(SEQ ID NO: 109)
EIVLTQSPATLSLSPGERATLSCRASQSVSIYLAWYQQKPGQAPR
LLIYDASNRATGIPARFSGSGSGTDFTLTISSLEPEDFAVYYCQQ
RSNWPPFTFGPGTKVDIKRTVAAPSVFIFPPSDEQLKSGTASVVC
LLNNFYPREAKVQWKVDNALQSGNSQESVTEQDSKDSTYSLSSTL
TLSKADYEKHKVYACEVTHQGLSSPVTKSFNRGEC

### Example 2. PTK7 Expression in Diseased Human Tissues

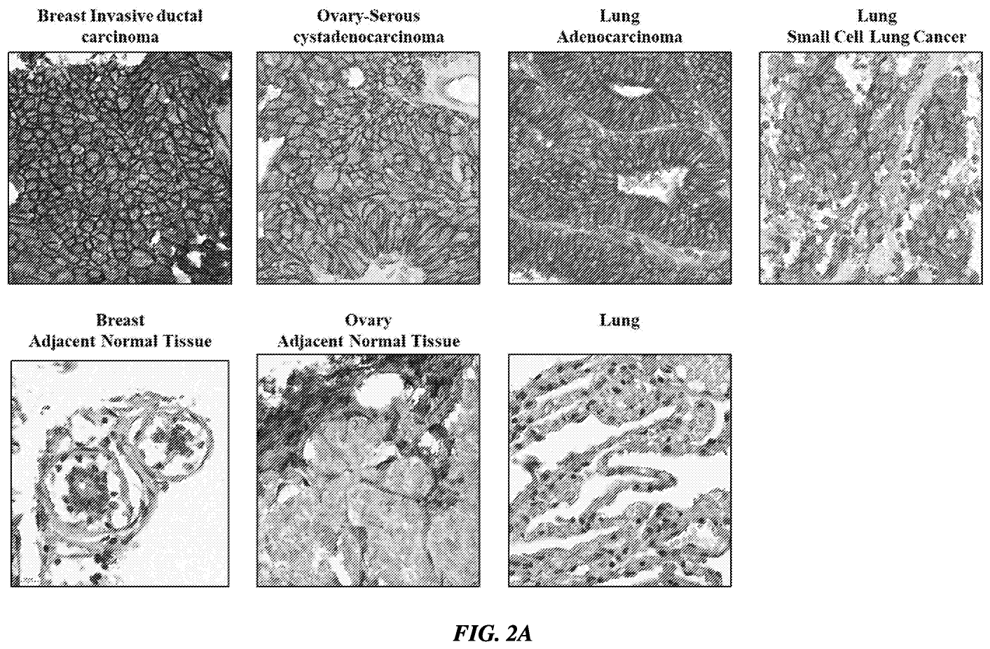

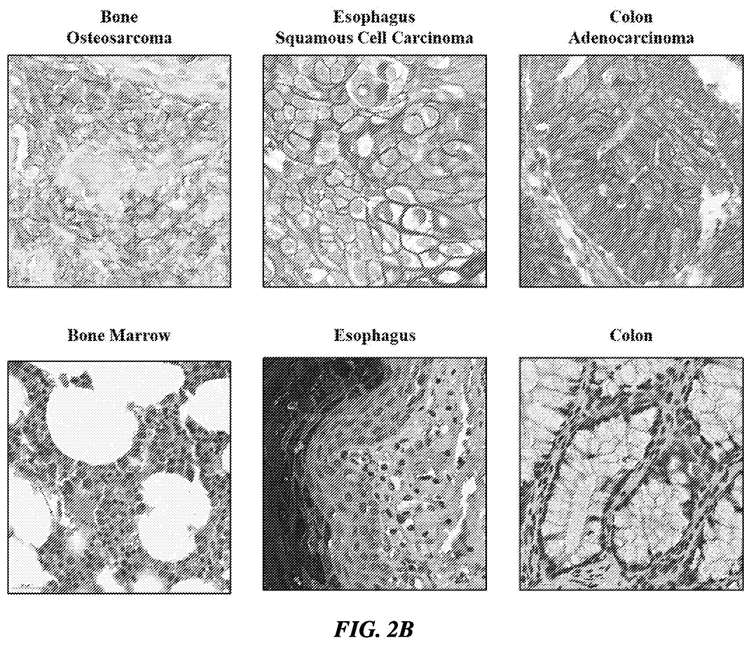

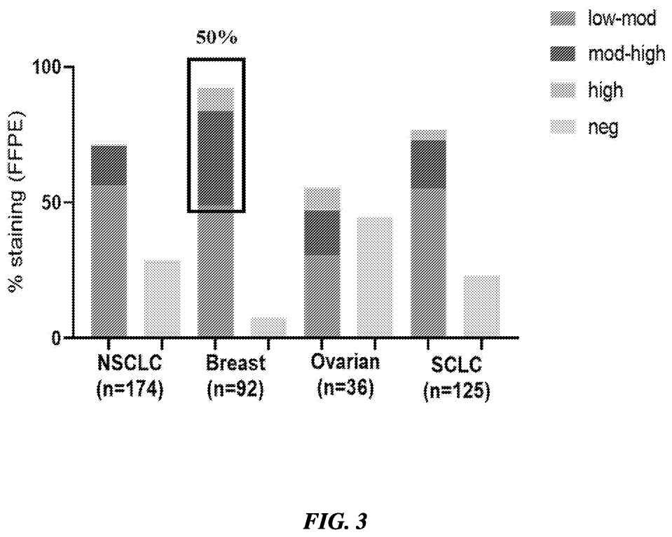

PTK7 expression was evaluated on formalin-fixed, paraffin-embedded (FFPE) diseased and normal patient tumor micro arrays using a mouse monoclonal anti-human PTK7 antibody, clone 4F9 (EMD Millipore, MABN721) (FIGS. 2A and 2B). Table 6 lists the FFPE tissue microarrays (TMA) used from US Biomax, Inc. FFPE sections were baked for 30 min at 60° C., deparaffinized and rehydrated. Antigen retrieval was performed for 40 min at 95° C. in 1× Reveal Decloaker solution using a Decloaker chamber (BioCare Medical). The remaining steps were performed at ambient temperature. Blocking to minimize non-specific staining was performed with Peroxidased 1 (BioCare Medical, PX968M) and Background Sniper (BioCare Medical, BS966M). Slides were stained for 60 min with the primary monoclonal anti-PTK7 antibody or mouse IgG isotype control (Novus Biologicals, LLC), washed and then stained for 30 min, with the secondary EnVision goat anti-mouse horseradish peroxidase antibody (Dako, K400111-2). Visualization of target antigen was visualized with DAB (brown color) substrate chromogen (Dako, K3468). Mayer's Hematoxylin (Dako, S3309) was used to counterstain the cell nuclei. Slides were scanned with the Pannoramic™ MIDI II scanner (3DHistech, ThermoFisher Scientific) and scored using semi-quantitative scoring system evaluating both the intensity of staining (1+-3+, where 1+ represents low antigen expression) as well as the percentage of the section stained (1-100%). Results were tabulated to establish patient prevalence summary data (FIG. 3). PTK7 was shown to be expressed in a broad range of solid tumor cancers.

TABLE 6
FFPE TMAs

| | FFPE TMA (US Biomax, |
|---|---|
| Tissue | Inc. Name/Number) |
| Brain glioblastoma | GL806f |
| Hepatocellular carcinoma | LV631 |
| Liver hepato- | LV642 |
| cholangiocarcinoma | |
| Osteosarcoma | OS804c |
| Gastric cancer | ST483e |
| Ovarian Cancer | OVC962 |
| Esophagus squamous | HEso-Squ127Lym-01 |
| cell carcinoma | |
| Advanced stage | PA1921a |
| pancreas cancer | |
| Lung adenocarcinoma | BCS04017b |
| Lung squamous | HLug-Squ090Lym-01 |
| cell carcinoma | |
| Lung small | BS04116 |
| cell cancer | |
| Intrahepatic | HIBD-Ade100PG-01 |
| biliary cancer | |

### Example 3. Human/Mouse Cross Reactivity of Anti-PTK7 CTX181

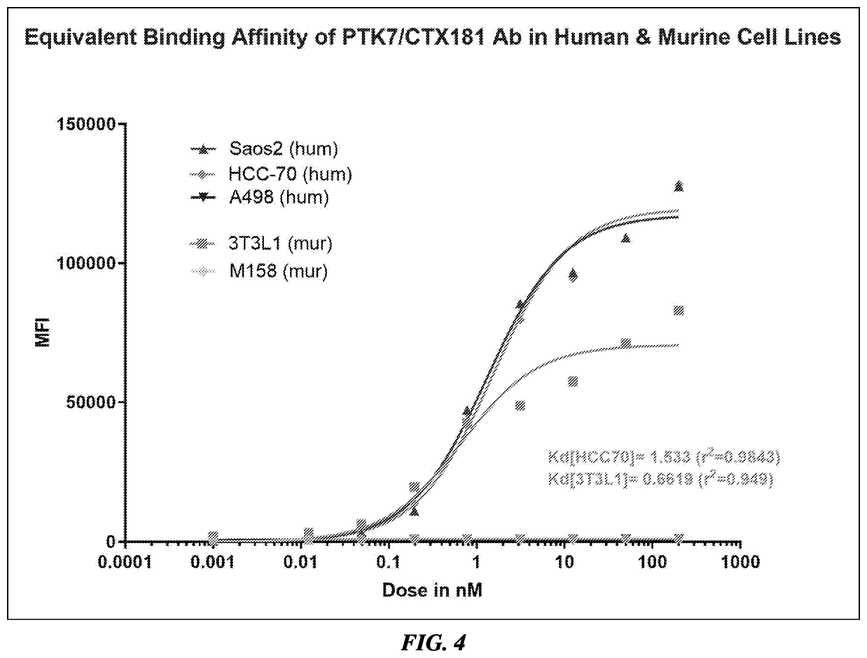

To evaluate the species cross reactivity of the custom CTX181 antibody, its binding affinity was assessed in murine 3T3L1 fibroblast and murine M158 breast cancer cell lines and compared to its binding affinity in human Saos2 (osteosarcoma), A498 (renal carcinoma) and HCC70 (breast cancer) cell lines. For each cell line, a dose titration binding assay (200 nM-0.0032 nM final CTX181 antibody) was run on 2×10⁶ cells at each concentration. Cells were stained 30 min on ice with CTX181 antibody, followed by washing and 15 min stain at RT with secondary APC fluorophore conjugated human IgG-Fc antibody. Cells were assessed for staining on a NovoCyte® flow cytometer (Acea Biosciences). FIG. 4 shows equivalent binding affinity for CTX181 antibody in murine and human cell lines.

### Example 4. PTK7 Expression in Normal Human and Mouse Tissues

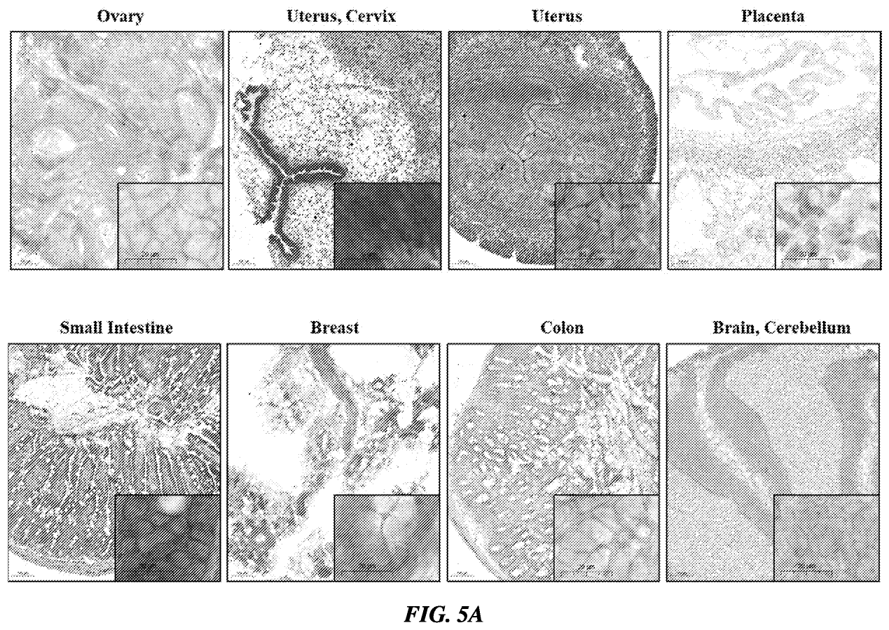

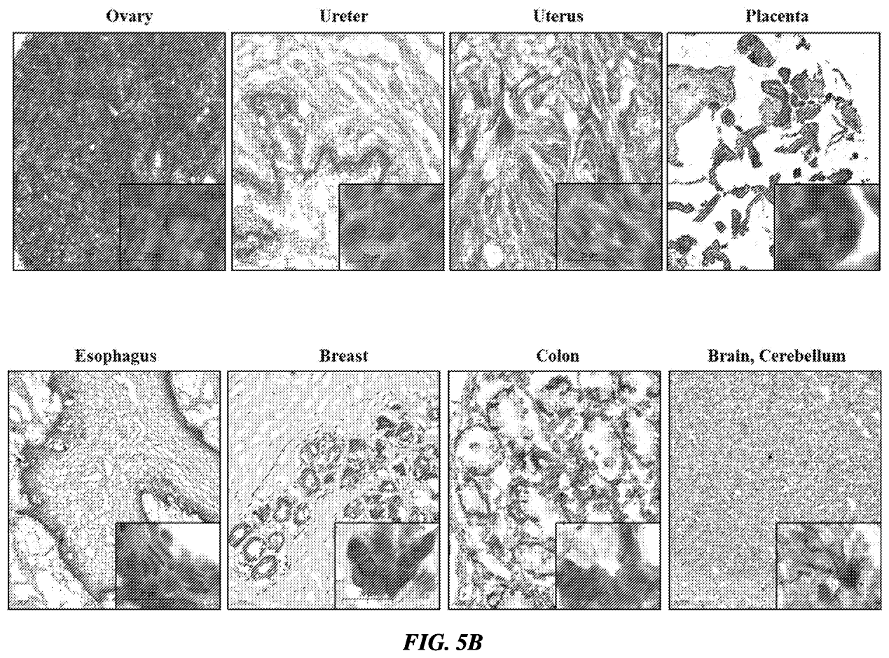

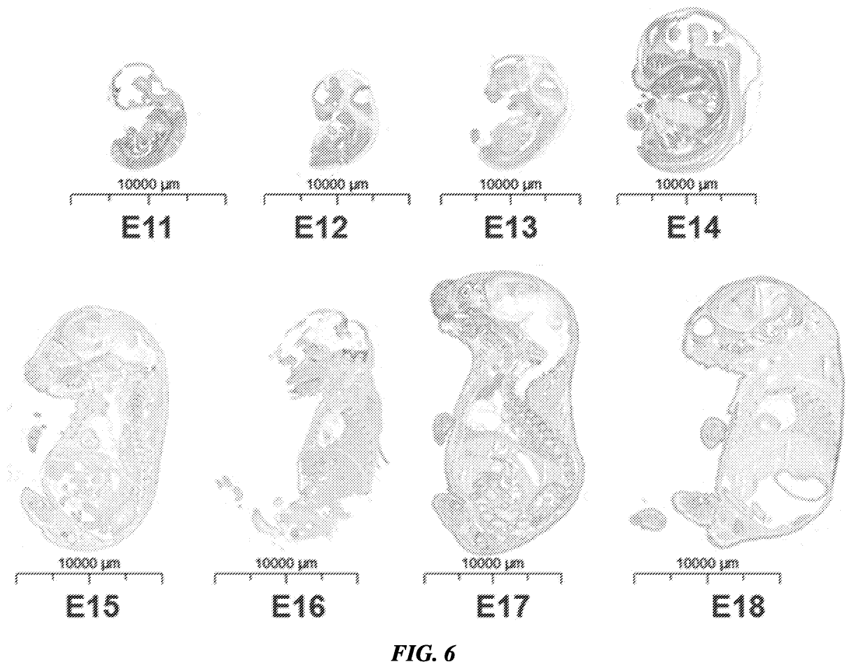

PTK7 expression was evaluated on a frozen normal mouse tissue panel (FDA Standard, Biochain; FIGS. 5A and 5B) as well as a frozen murine embryonic development array (Zyagen; FIG. 6) using CTX181, specific to the PTK7 CAR construct, or a biotinylated mouse isotype control (Novus, NBP2-21948). Slides were fixed with −20° C. acetone for 10 min at ambient temperature followed by a manual immunohistochemical staining at ambient temperature. Blocking to minimize non-specific staining was performed with Peroxidased 1, Avidin and Biotin and Background Sniper (BioCare Medical, PX968M, AB972L, BS966M) in sequential steps. Slides were stained with the biotinylated CTX181 primary monoclonal antibody (1:300 for human; 1:600 for murine) for 30 min followed by incubation with 4plus Streptavidin-HRP Label (BioCare Medical) reagent for 15 min each. Visualization of target antigen was visualized with DAB (brown color) substrate chromogen (Dako, K3468). Mayer's Hematoxylin (Dako, S3309) was used to counterstain the cell nuclei.

### Example 5. Generation of TRAC⁻/β2M⁻/Anti-PTK7 CAR⁺ T Cells

This example describes the production of allogeneic human T cells that lack expression of the T cell receptor (TCR) gene (gene edited in the TCR Alpha Constant (TRAC) region), the β2-microglobulin (β2M) gene, and that express a chimeric antigen receptor (CAR) targeting protein tyrosine kinase 7 (PTK7) and PTK7⁺ cancers. Four unique anti-PTK7 CARs (PTK7-4, PTK7-7, PTK7-13, and PTK7-17) comprising CD28 co-stimulatory domains were separately expressed in TRAC⁻/β2M⁻ T cells for experimentation and evaluation. The PTK7-4 was also generated with a 41BB co-stimulatory domain in place of CD28 (PTK7-4b). Table 7 lists the PTK7 CAR structures. Table 11 lists the PTK7 CAR component sequences. Table 12 lists the donor components.

TABLE 7

| CAR | CAR structure | SEQ ID NO: |
|---|---|---|
| PTK7-4 | CD8[signal peptide]-VH-linker-VL- | 50 |
| | CD8[tm]-CD28[co-stimulatory | |
| | domain]-CD3ζ | |
| PTK7-4b | CD8[signal peptide]-VH-linker-VL- | 52 |
| | CD8[tm]-41BB[co-stimulatory | |
| | domain]-CD3ζ | |
| PTK7-7 | CD8[signal peptide]-VL-linker-VH- | 66 |
| | CD8[tm]-CD28[co-stimulatory | |
| | domain]-CD3ζ | |
| PTK7-13 | CD8[signal peptide]-VL-linker-VH- | 73 |
| | CD8[tm]-CD28[co-stimulatory | |
| | domain]-CD3ζ | |
| PTK7-17 | CD8[signal peptide]-VH-linker-VL- | 80 |
| | CD8[tm]-CD28[co-stimulatory | |
| | domain]-CD3ζ | |

Activated primary human T cells were electroporated with Cas9:gRNA RNP complexes and adeno-associated adenoviral vectors (AAVs) to generate TRAC⁻/β2M⁻/anti-PTK7 CAR⁺ T cells. Recombinant AAV serotype 6 (AAV6) comprising one of the nucleotide sequences encoding an anti-PTK7 CAR (SEQ ID NOs: 49, 51, 65, 72, 79, or 112) were delivered with Cas9:sgRNA RNPs (1 μM Cas9, 5 μM gRNA) to activated allogeneic human T cells. The following sgRNAs were used: TRAC (SEQ ID NO: 28) and β2M (SEQ ID NO: 30). The unmodified versions (or other modified versions) of the gRNAs may also be used (e.g., SEQ ID NO: 18 or SEQ ID NO: 20). See also Table 4.

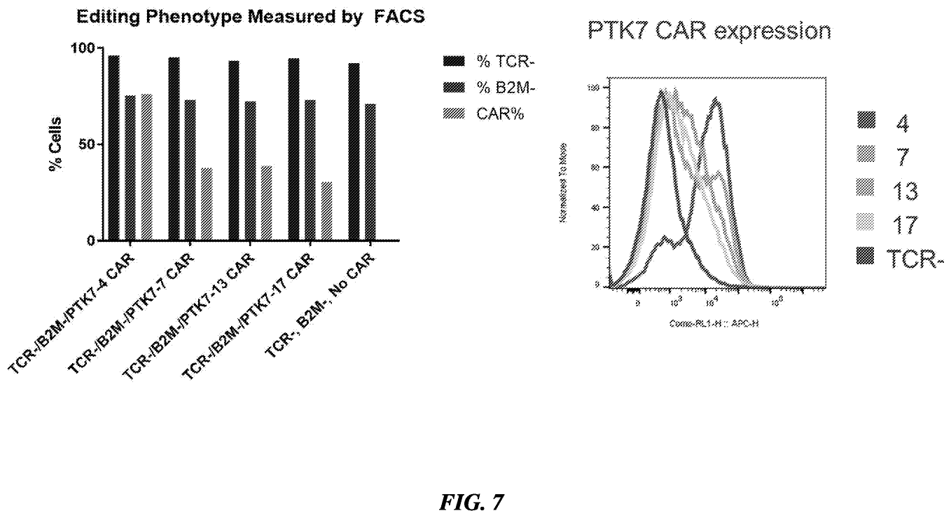

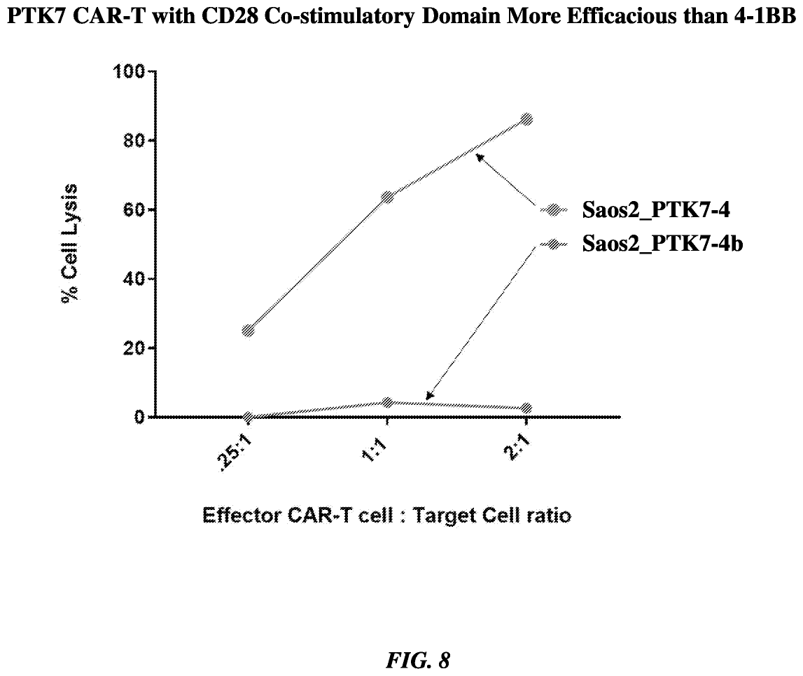

About one (1) week post electroporation, cells were processed for flow cytometry to assess TRAC, β2M, and anti-PTK7 CAR expression levels at the cell surface of the edited cell population (FIG. 7). Table 8 list the antibodies that were used. For all anti-PTK7 CAR T cells and TRAC⁻/β2M⁻ control cells, >90% of viable cells lacked expression of TCR and >60% lacked expression of β2M. The cells treated with the construct encoding the PTK7-4 CAR had the highest percentage of viable cells expressing an anti-PTK7 CAR⁺ (>70%). The orientation of the VH and VL sequences in the scFV appear to effects expression of the CAR. The PTK7-4 CAR differs from the PTK7-7 CAR in the orientation of the VH and VL sequence, however, PTK7-7 only expressed in <50% of the viable cell population. The PTK7 CAR T cell with the CD28 co-stimulatory domain (PTK7-4) was more efficacious than the 4-1BB co-stimulatory domain (PTK7-4b) (FIG. 8).

TABLE 8

| Antibody | Clone | Fluor | Catalogue # | Dilution |
|---|---|---|---|---|
| TCRαβ | BW242/412 | PE | 130-099-661 (Miltenyi) | 1:100 |
| β2M | 2M2 | FITC | 316304 (Biolegend) | 1:100 |
| IgG, F(ab′)₂ | polyclonal | Biotinylated; | 109-006-097 (Jackson | 1:20 |
| fragment | | Detected | Immuno-research) | |
| specific | | with SA-APC | | |
| Streptavidin- | N/A | APC | 17-4317-82 | 1:100 |
| APC (SA-APC) | | | (eBioscience through | |
| | | | ThermoFisher) | |

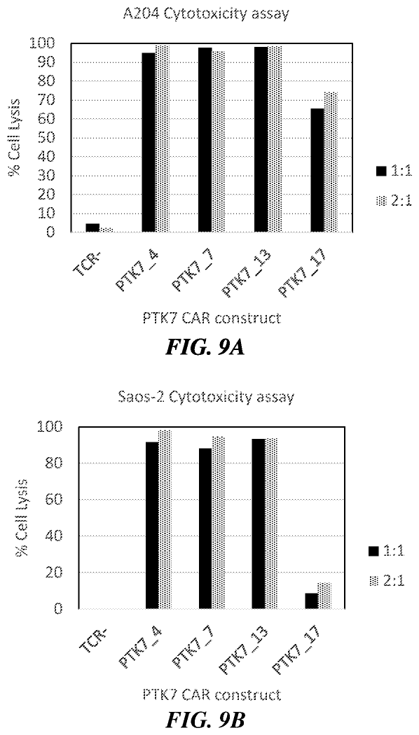

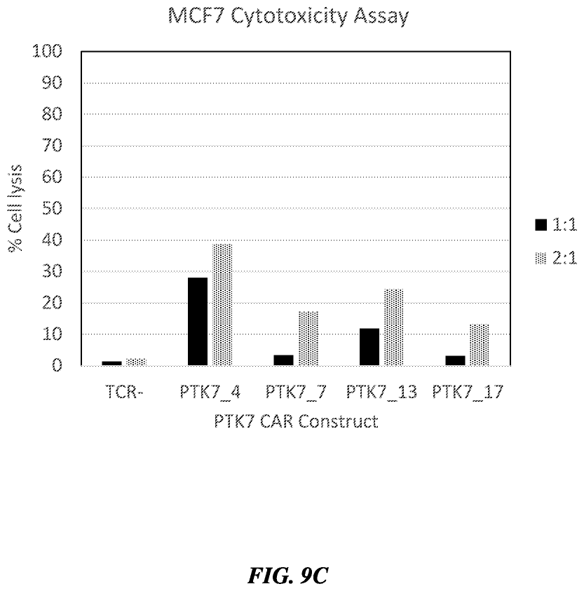

Cell Kill Assay. A cell killing (cytotoxicity) assay was then used to assess the ability of the TRAC⁻/β2M⁻/anti-PTK7 CAR⁺ T cells to cause cellular lysis in adherent sarcoma cell lines that express PTK7 (A-204 and Saos-2) and a breast cancer cell line that expresses PTK7 (MCF7). Adherent cells were seeded in 96-well plates at 50,000 cells per well and left overnight at 37° C. During the following day, T cells were added to the wells containing target cells at ratios of 2:1 or 1:1 T cell:target cell. TRAC⁻/β2M⁻ T cells were used as a negative control. After approximately 20 hours, T cells were removed from the culture by aspiration and 100 μL Cell titer-Glo (Promega) was added to each well of the plate to assess the number of remaining viable cells. The amount of light emitted from each well was then quantified using a plate reader. The anti-PTK7 CAR T cells, particularly those expressing the PTK7-4, PTK7-7 and PTK7-13 constructs, exhibited potent cytotoxicity towards A-204 (FIG. 9A) and Saos (FIG. 9B) cell lines. Further, the anti-PTK7 CAR T cells expressing the PTK7-4 CAR showed the highest cytotoxic activity towards the MCF7 cell line (FIG. 9C), which is known to have lower PTK7 mRNA expression than the tested sarcoma cell lines.

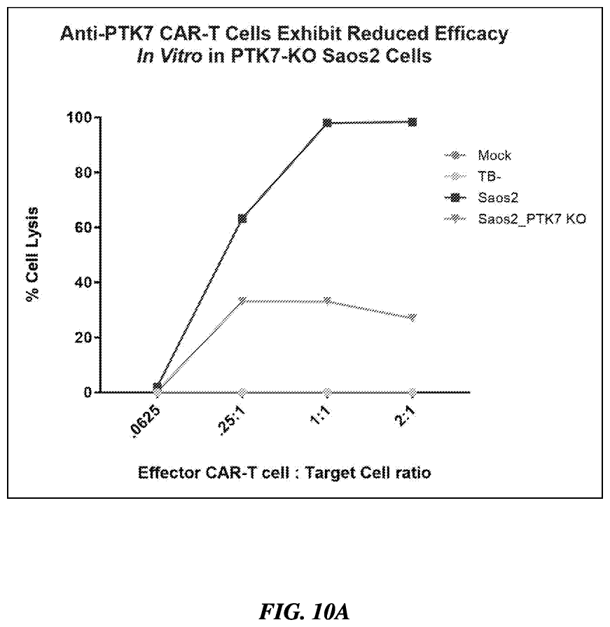

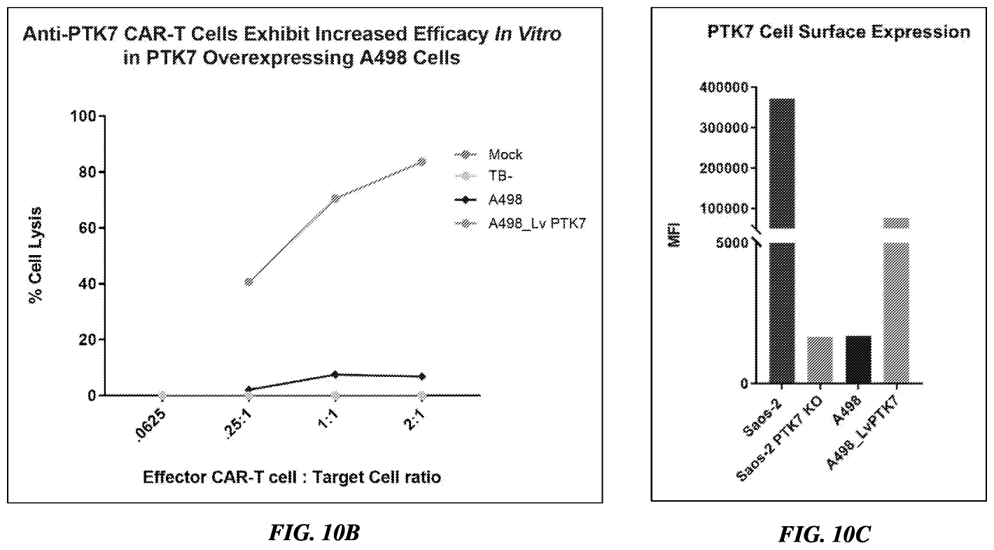

The cell specificity of the PTK7-4 CAR T cells was exemplified using PTK7 knock-out (KO) Saos2 cells (FIG. 10A) and PTK7 overexpressing A498 cells (FIG. 10B). PTK7 KO Saos-2 cells were generated via electroporation of ribonucleoprotein particle (RNP) complexes (1 μM Cas9 and 5 μM PTK7 gRNA (SEQ ID NO: 111; see Table 9)) according to established methods. Cells were analyzed for loss of PTK7 cell surface expression by flow cytometry using the CTX181 mAb (FIG. 10C) and subsequently expanded. Flow cytometry analysis showed that protein expression was reduced by 88.6% indicating highly efficient gene editing. In vitro efficacy of PTK7 KO Saos-2 cells was evaluated in a cell cytotoxicity assay compared to Saos-2 WT cells. FIG. 10A show a decrease in efficacy of Saos2 PTK7 KO cells to be lysed by PTK7 CAR T cells, indicating the CAR T-cells were specific to PTK7 expressing target cells.

TABLE 9
PTK7 gRNA Sequence

| | Unmodified Sequence | |
|---|---|---|
| | (gRNA Spacer Sequence | |
| | underlined) | Modified Sequence |
| Name | (SEQ ID NO: 110) | (SEQ ID NO: 111) |
| PTK7_T11 | CCGCCGCGAUGGGAGCUGCGg | C*C*G*CCGCGAUGGGA |
| | uuuuagagcuagaaauagcaa | GCUGCGguuuuagagcu |
| | guuaaaauaaggcuaguccgu | agaaauagcaaguuaaa |
| | uaucaacuugaaaaaguggca | auaaggcuaguccguua |
| | ccgagucggugcUUUU | ucaacuugaaaaagugg |
| | | caccgagucggugcU* |
| | | U*U*U |

*: 2′-O-methyl phosphorothioate residue

PTK7 overexpressing A498 cells were generated as follows: A498 renal cell carcinoma cells were plated at 60-70% confluency in MEM1α+10% FBS media supplemented with 10 μg/ml polybrene. Based on the desired multiplicity of infection (MOI), A498 cells were transduced the next day with a lentivirus expressing humPTK7 cDNA (LPP-A6381-Lv225-200, GeneCopoeia, Rockville, MD). Following 24-48 hrs lentivirus infection, fresh media was replaced containing 4 μg/ml puromycin selection. Puromycin treatment was continued for 5-7 days post transduction until all non-transduced cells were eliminated from the culture. Expression of the humPTK7cDNA lentiviral construct was assessed by flow cytometry (FIG. 10C) using CTX181 mAb. In vitro efficacy of humPTK7 cDNA expressing A498 cells was evaluated in a cell cytotoxicity assay compared to A498 WT cells. FIG. 10B shows an increase in efficacy of PTK7 CAR T cells to lyse humPTK7 cDNA expressing A498 cells as compared to A498 WT cells, indicating the PTK7 CAR T-cells were specific to PTK7 antigen expressing target cells.

### Example 6. In Vitro Potency of PTK7 CAR T Cells in Solid Tumor Cell Lines

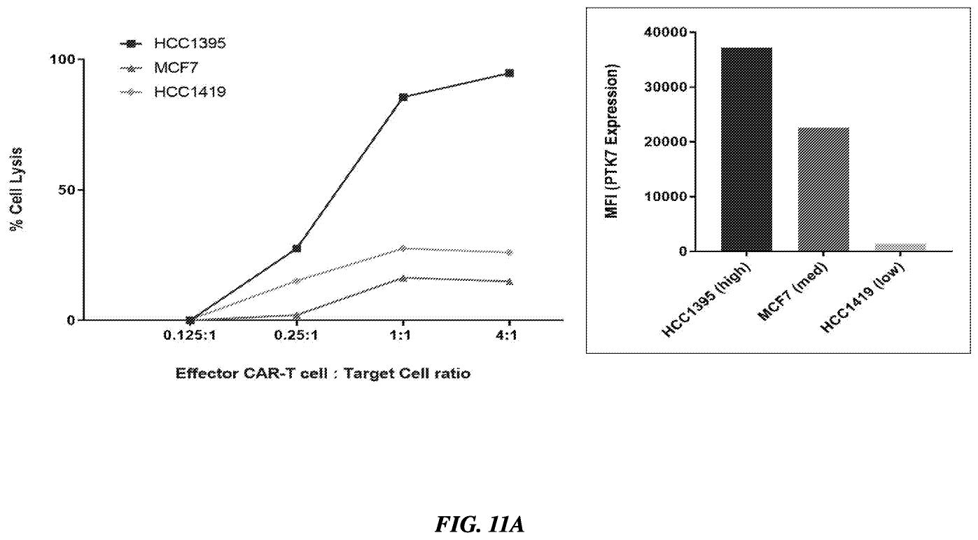

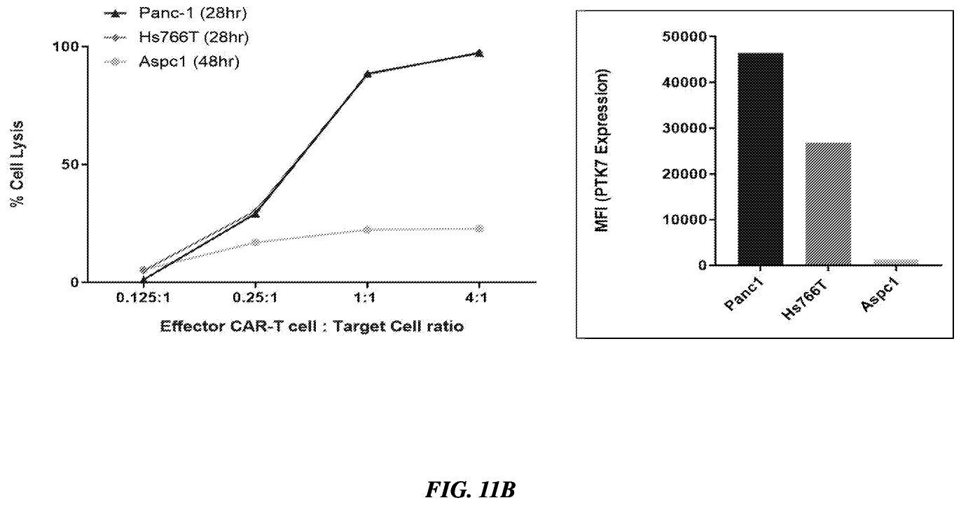

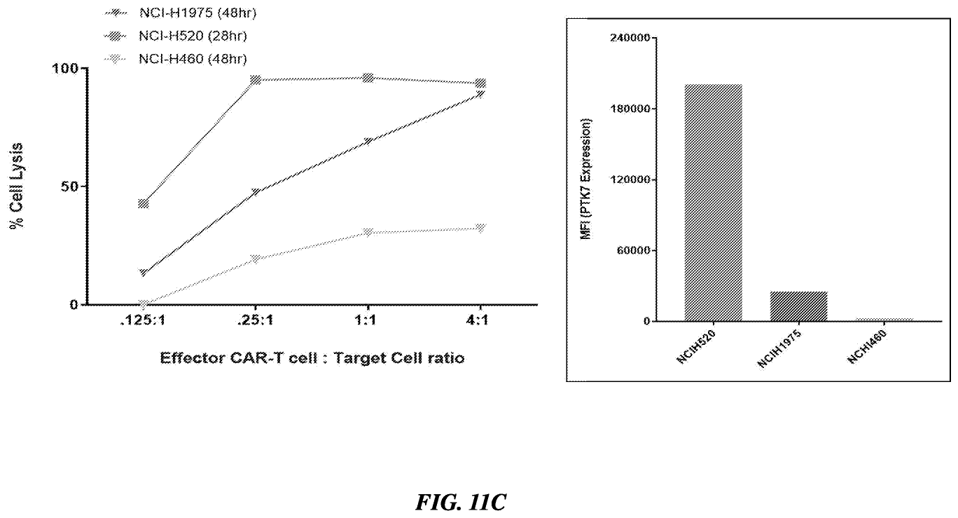

Cell Kill Assay. To examine the efficacy in additional tumor cell lines, high, med, low PTK7 expressing cell lines for breast, pancreatic and NSCL cancers were selected from the Broad Cancer Cell Line database. A cell killing (cytotoxicity) assay was used to assess the ability of the TRAC⁻/β2M⁻/anti-PTK7 CAR⁺ T cells to cause cellular lysis in an adherent sarcoma cell line that expresses PTK7 (Saos-2), breast cancer cell lines that expresses PTK7 to varying degrees (HCC1395, MCF7, HCC1419), pancreatic cell lines that express PTK7 to varying degrees (Panc-1, Hs766T, Aspc1) and non-small cell lung cancer cell lines that express PTK7 to varying degrees (NCI-H1975, NCI-H520, NCI-H460). Adherent cells were seeded in 96-well plates at 50,000 cells per well and left overnight at 37° C. During the following day, T cells were added to the wells containing target cells at ratios of 0.125:1, 0.25:1, 1:1 or 4:1 effector T cell:target cell. TRAC⁻/β2M⁻T cells were used as a negative control. After approximately 24 hours, T cells were removed from the culture by aspiration and 100 μL CellTiter-Glo® (Promega) was added to each well of the plate to assess the number of remaining viable cells. The amount of light emitted from each well was then quantified using a plate reader. PTK7 protein expression was assessed by staining target cells for 30 min on ice with the CTX181 antibody, followed by washing and a 15 min stain at RT with secondary APC fluorophore conjugated human IgG-Fc antibody. Target cells were assessed for staining on a Novocyte® flow cytometer (Acea Biosciences). FIGS. 11A-11C show that the anti-PTK7 CAR T cells exhibited cytotoxicity towards all cell lines tested and their in vitro potency trended with the level of PTK7 expression in these tumor cell lines.

Functional activity of PTK7 CAR T cells was further assessed using cytokine release assays for Interferon gamma (IFNγ) and Interleukin-2 (IL2). T cells of all tested genotypes were incubated with target cells for 24 hours at cellular ratios indicated above. After 24 hours, supernatant media surrounding a cellular sample was collected and the levels of IFNγ and IL2 were measured using an ELISA (RD Systems) following the manufacturer's instructions. PTK7 CAR T cells secreted IFNγ and IL2 in the presence of PTK7 expressing cancer cell lines (Saos2, HCC1395, MCF7, Panc1, Hs766T, NCI-H520 and NCI-H1975) when used at a 4:1 or 1:1 T cell:target cell ratio. The control cells (TCR⁻/β2M⁻ (No AAV) and non-edited (no RNP)) showed no specific IFNγ or IL2 secretory response in the presence of any of the cancer cell lines listed. Collectively, these functional assays demonstrated that anti-PTK7 CAR T cells were cytotoxic towards and secreted IFNγ and IL2 in the presence of cells that were expressing PTK7.

### Example 7. Functional Capabilities of Anti-PTK7 CAR T Cells

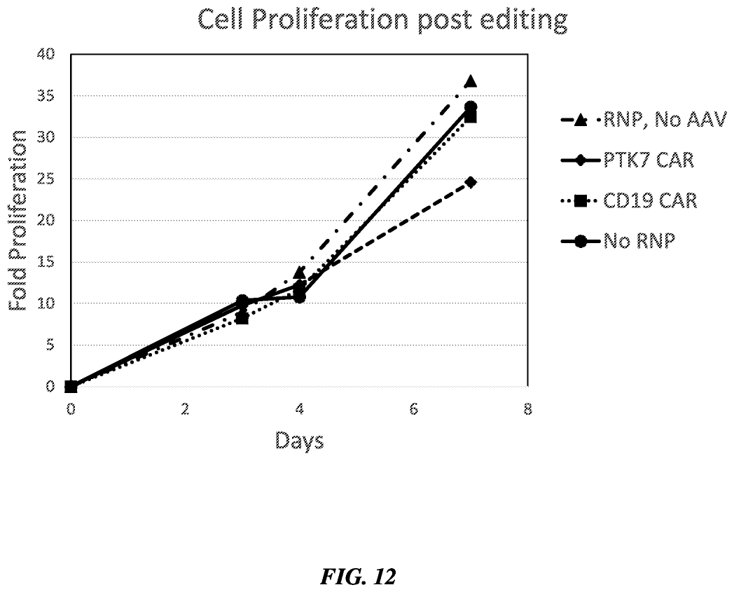

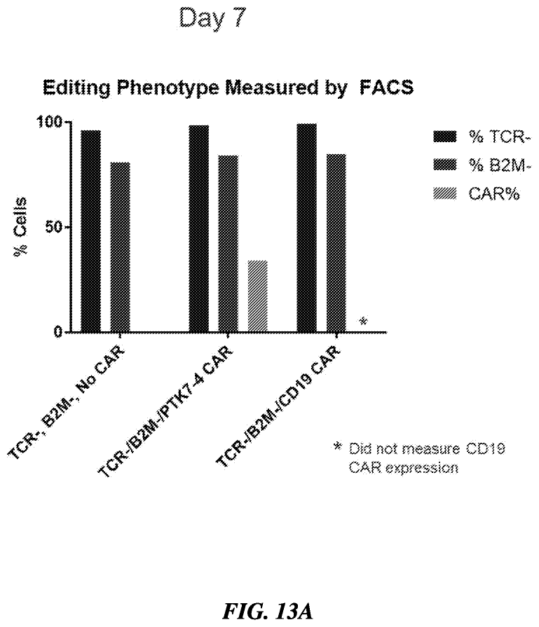

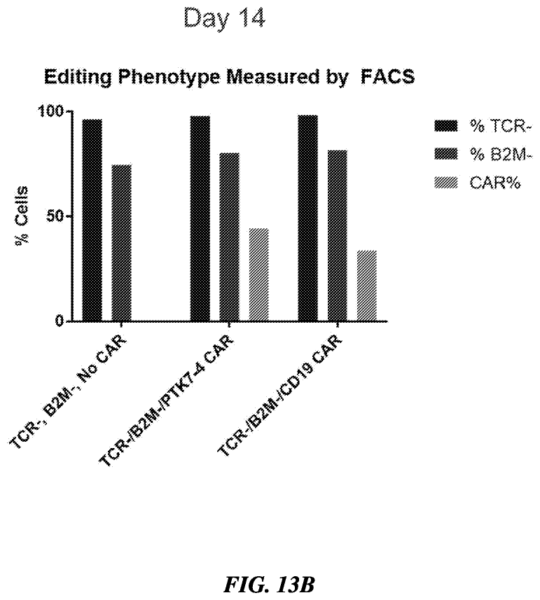

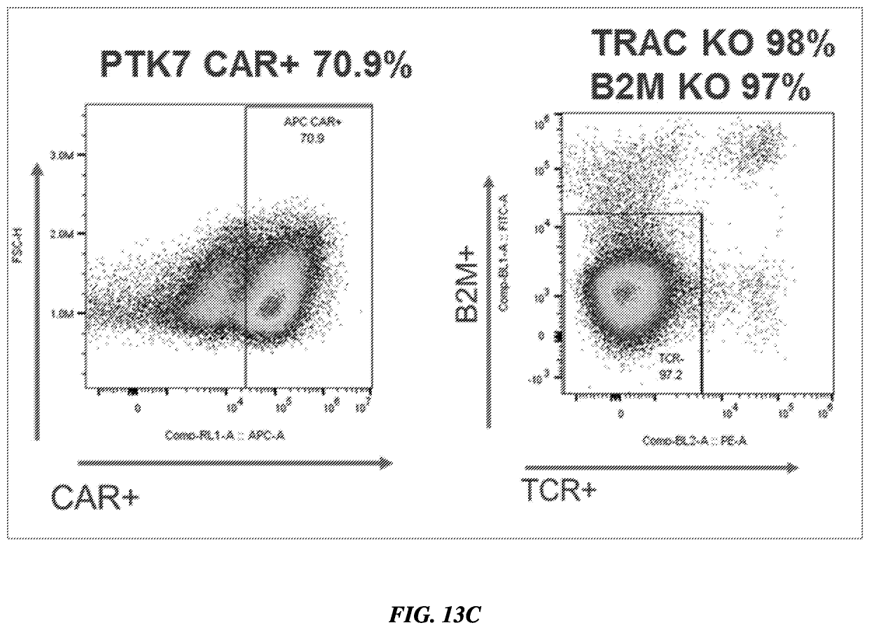

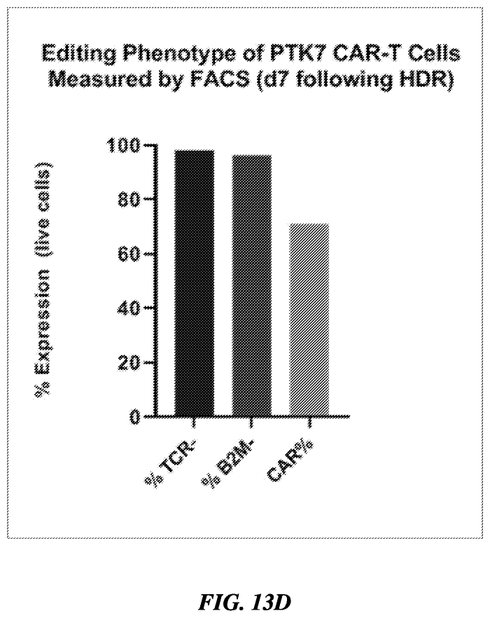

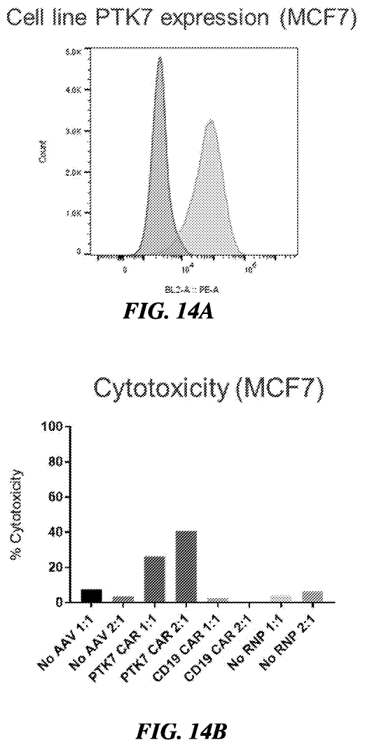

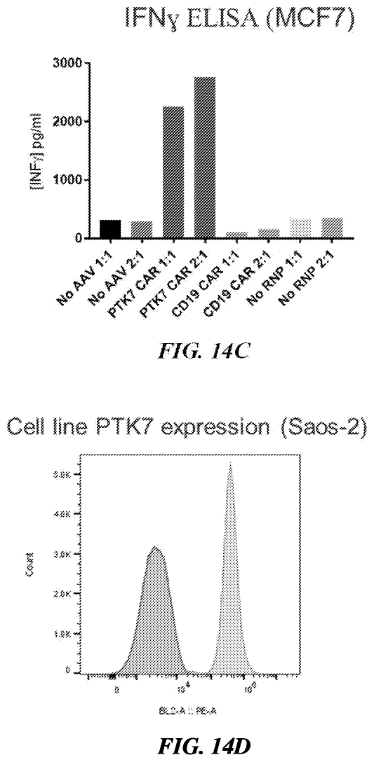

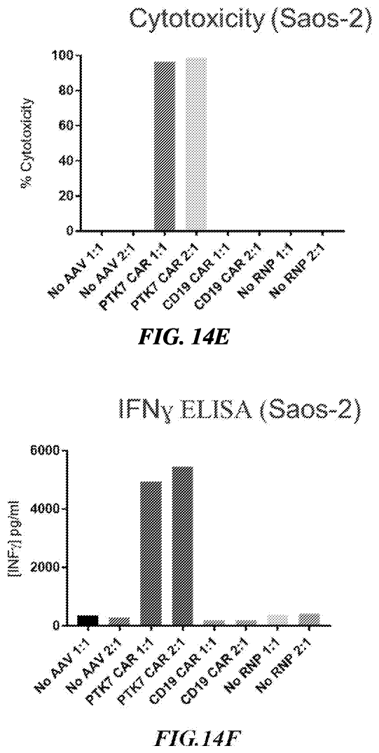

TRAC⁻/β2M⁻/anti-PTK7-4 CAR⁺ T cells (also referred to as PTK7-4 CAR T cells) were generated as described in Example 5. Populations of TRAC⁻/β2M⁻/anti-CD19 CAR⁺ T cells and TRAC⁻/β2M⁻ T cells were similarly generated for use as controls. Following preparation of the edited T cells by transfection, the ability of all edited cell types, and non-edited T cells, to proliferate over the course of 7 days was tested. 5×10⁶ cells were plated for each genotype of T cells. Notably, as shown in FIG. 12, TRAC⁻/β2M⁻/anti-PTK7-4 CAR⁺ T cells were able to proliferate at rates and levels comparable to all control experiments. After 7 days of cellular proliferation, a sample of each genotype to be injected into mouse models were frozen in cyrostor10 and stored in liquid nitrogen. The remaining cells of each genotype were allowed to continue to proliferate until day 14 post-editing. Notably, as shown in FIGS. 13A-13B, the percentage of viable cells with genetic editing of TCR, β2M, and CAR remained consistent from Day 7 to Day 14 post-editing. FIGS. 13C-13D show that in subsequent experiments, 70.9% of the cells expressed the PTK7-4 CAR construct while 98% of the cells had the TRAC KO and 97% of the cells had the β2M KO. The expression of anti-CD19 CAR was detected using a biotinylated polyclonal anti-mouse FAB primary followed by a streptavidin-APC conjugate; the expression of anti-PTK7 CAR was detected using an anti-PTK7 antibody-PE conjugate (Miltenyi cat #: 130-091-364) (FIGS. 14A and 14D).

The functional activity of the PTK7-4 CAR T cells was verified using an adherent cytotoxicity assay as described in Example 5. PTK7-4 CAR T cells (PTK7 CAR) were capable of causing cytotoxicity of PTK7 expressing Saos-2 and MCF7 cells when used at a 1:1 or 2:1 T cell:target cell ratio. The control cells (TCR⁻/β2M⁻ (No AAV); TCR⁻/β2M⁻/anti-CD19 CAR (CD19 CAR); and non-edited (no RNP)) showed no specific cytotoxicity against either Saos-2 or MCF7 cells (FIGS. 14B and 14E).

Functional activity of PTK7-4 CAR T cells was further assessed using a cytokine (Interferon gamma/IFNγ) release assay. T cells of all tested genotypes were incubated with target cells (Saos-2 and MCF7 cells) for 24 hours at cellular ratios of 1:1 and 2:1. After 24 hours, supernatant media surrounding a cellular sample was collected and the levels of IFNγ were measured using an ELISA (RD Systems) following the manufacturer's instructions PTK7-4 CAR T cells (PTK7 CAR) secreted IFNγ in the presence of PTK7 expressing Saos-2 and MCF7 cells when used at a 1:1 or 2:1 T cell:target cell ratio. The control cells (TCR⁻/β2M⁻ (No AAV); TCR⁻/β2M⁻/anti-CD19 CAR⁺ (CD19 CAR); and non-edited (no RNP)) showed no specific IFNγ secretory response in the presence of either Saos-2 or MCF7 cells (FIGS. 14C and 14F).

Collectively, these functional assays demonstrated that anti-PTK7 CAR T cells were cytotoxic towards and secreted IFNγ in the presence of cells that are expressing PTK7.

### Example 8. In Vitro Cytotoxicity Assay

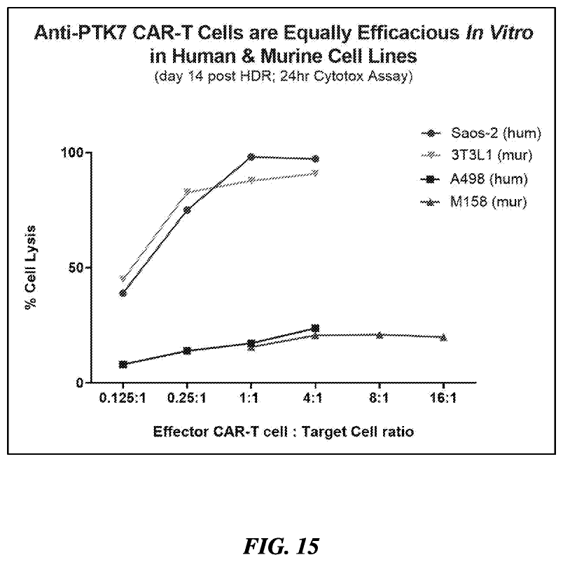

To evaluate the efficacy in vitro of anti-PTK7 CAR T cells against murine cells, 3T3L1 fibroblast and M158 breast cancer cells, a 24 hour cytotoxicity assay was performed. Results were compared to efficacy of anti-PTK7 CAR T cells to lyse human Saos2 osteosarcoma and human A498 renal cell carcinoma cell lines. Adherent cells were seeded in 96-well plates at 50,000 cells per well and left overnight at 37° C. During the following day, T cells were added to the wells containing target cells at ratios of 0.125:1, 0.25:1, 1:1 or 4:1 effector T cell:target cell. TRAC⁻/β2M⁻ T cells were used as a negative control. After approximately 24 hours, T cells were removed from the culture by aspiration and 100 μL CellTiter-Glo® (Promega) was added to each well of the plate to assess the number of remaining viable cells. The amount of light emitted from each well was then quantified using a plate reader. The anti-PTK7 CAR T cells exhibited cytotoxicity equally well towards human Saos2 and murine 3T3L1 cell lines (FIG. 15). Conversely, human A498 and murine M158 cells did not lyse in the presence of anti-PTK7 CAR T cells since PTK7 expression levels are low in these 2 cell lines.

### Example 9. Tolerability of Anti-PTK7 CAR T Cells in Mouse Models

The ability of NOG mice to tolerate treatment via injection with TRAC⁻/β2M⁻/anti-PTK7-4 CAR⁺ T cells was tested. Two NOG mice were dosed with 10 million TRAC⁻/β2M^{—}/anti-PTK7-4 CAR T cells (generated as previously described above); two additional mice were dosed with 10 million anti-CD19 CAR T cells. All CAR T cells were administered via tail vein injection.

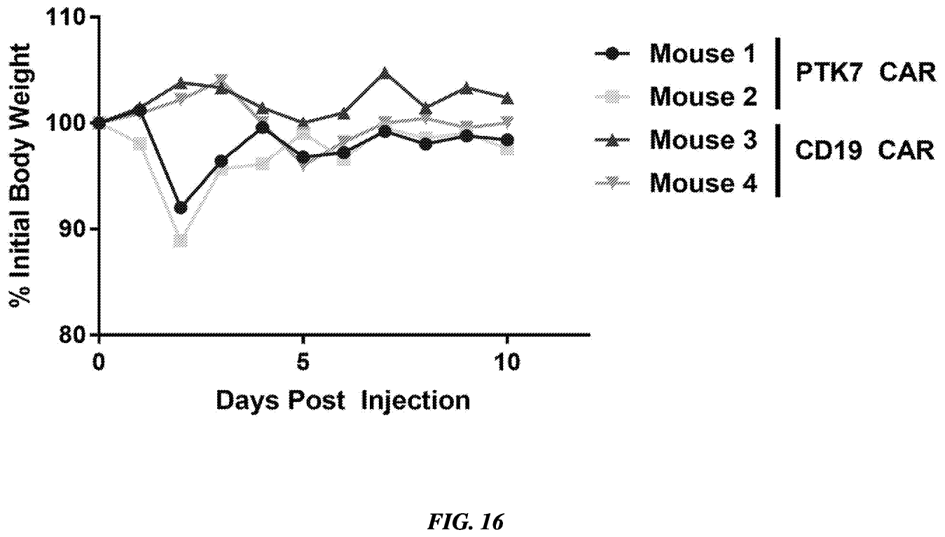

Mice were weighed daily and monitored for distress or moribundity. Mice treated with anti-PTK7 CAR T cells showed minimal weight loss similar to mice treated with anti-CD19 CAR T cells (FIG. 16). Following a period of ten days post injection, the animals were sacrificed, and spleens and blood samples were analyzed for the presence of human T cells (human CD45+ cells). In both the spleen and blood, mice treated with anti-PTK7 CAR T had higher levels of edited CAR T cells (huCD45+ cells) than in mice treated with anti-CD19 CAR T cells (Table 10), suggesting that the anti-PTK7 CAR T cells will expand in the presence of the mouse antigen.

TABLE 10
Percent humanCD45+ cells ((huCD45+/muCD45+)*100)
in PTK7 and CD19 CAR T cell treated mice

| | PTK7 | | CD19 | |
|---|---|---|---|---|
| | Mouse 1 | Mouse 2 | Mouse 1 | Mouse 2 |
| Spleen | 8.56 | 10.01 | 0.58 | 0.82 |
| Blood | 18.47 | 15.13 | 0.42 | 0.93 |

Collectively, the transient body weight loss and higher levels of CAR T cells in the anti-PTK7 CAR treated mice suggested that the anti-PTK7 CAR recognized an antigen in the mouse, which resulted in CAR T cell proliferation. A general lack of significant toxicities was surprising and indicates that the known on-target/off-tissue toxicities associated with targeting PTK7 are tolerable in mice and may further be tolerable in humans.

### Example 10. In Vivo Efficacy of Anti-PTK7 CAR-T Cells in Xenograft Mouse Models

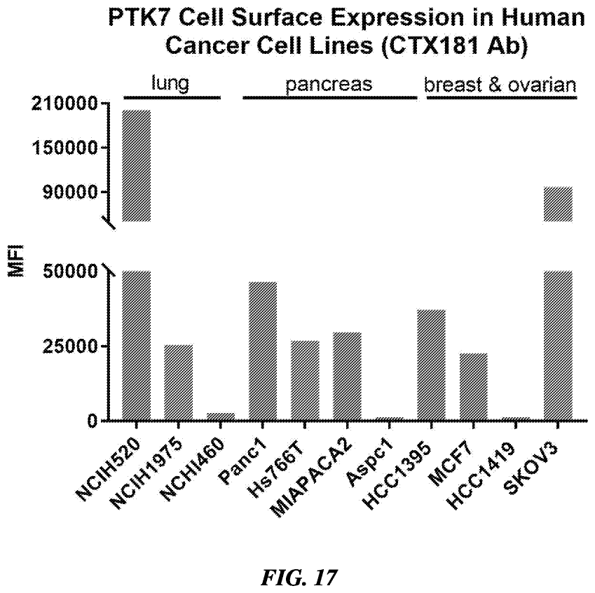

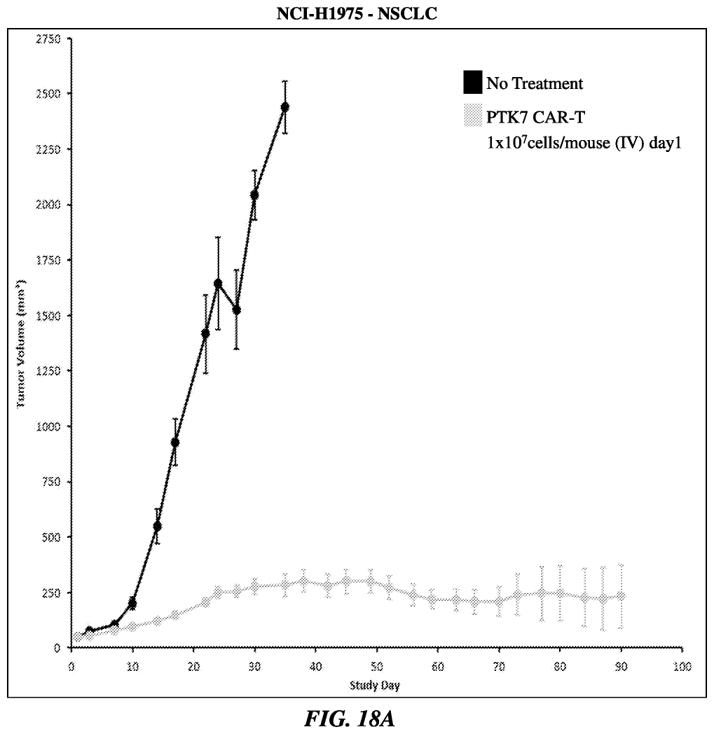

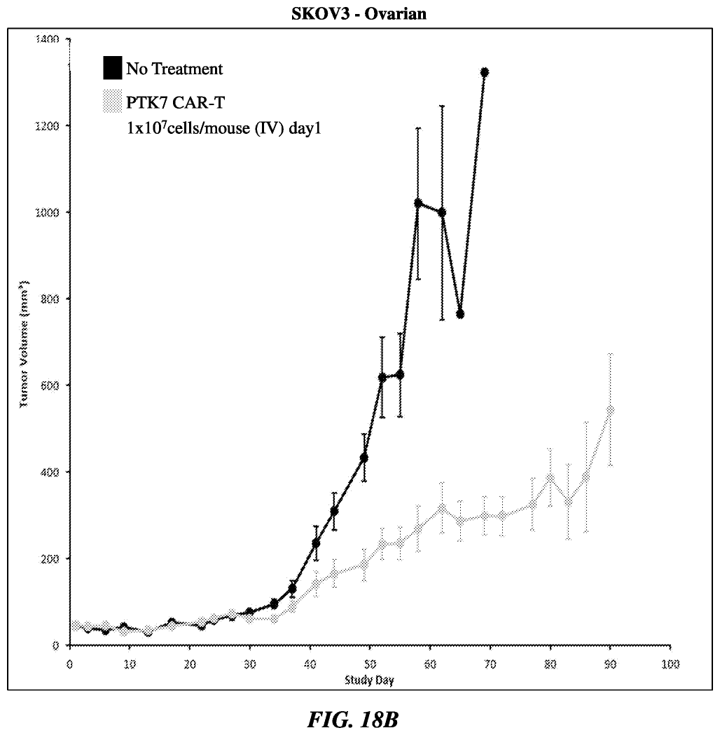

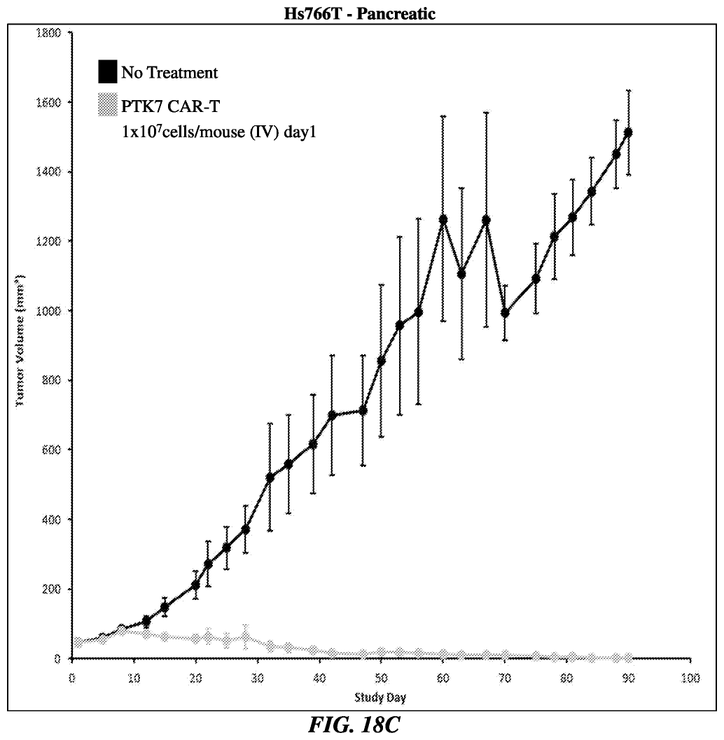

The efficacy of anti-PTK7 CAR-T cells was tested in vivo in SKOV-3 human ovarian, NCI-H1975 human non-small cell lung cancer and human pancreatic Hs766T tumor xenograft mouse models. FIG. 17 shows the PTK7 cell surface expression levels in human cancer cell lines using the CTX181 Ab. For each xenograft model, 5 female (5-8 weeks) NOG mice were dosed single time point IV, single dose (5×10⁷ cells/ml) TRAC⁻/β2M⁻/anti-PTK7 CAR T cells (generated as previously described above). Body weight (2× weekly) and tumor volume were the endpoints measured throughout course of the study. Mice were dosed with anti-PTK7 CAR T cells when tumors (cell lines injected subcutaneous into right flank) reached 50 mm. Studies were terminated when tumors reached endpoint size (1000 mm for SKOV3 (ovarian), 2000 mm for NCI-H1975 (NSCLC) and Hs766T (pancreatic)) or 90 days, whichever occurred first. Mice were housed and monitored under pathogen free conditions and IACUC standards. Anti-PTK7 CAR-T cells were efficacious in all three xenograft models (NCI-H1975 non-small cell lung cancer xenograft model (FIG. 18A), SKOV3 ovarian cancer xenograft model (FIG. 18B), and Hs766T pancreatic cancer xenograft model (FIG. 18C)).

### Example 11. In Vivo Efficacy of Anti-PTK7 CAR-T Cells in Xenograft Mouse Models

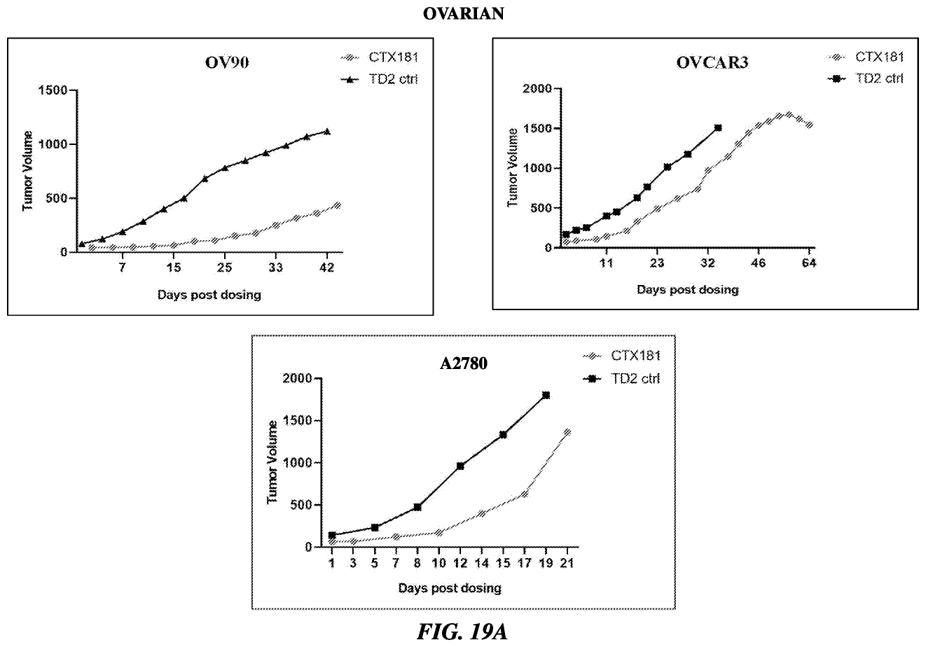

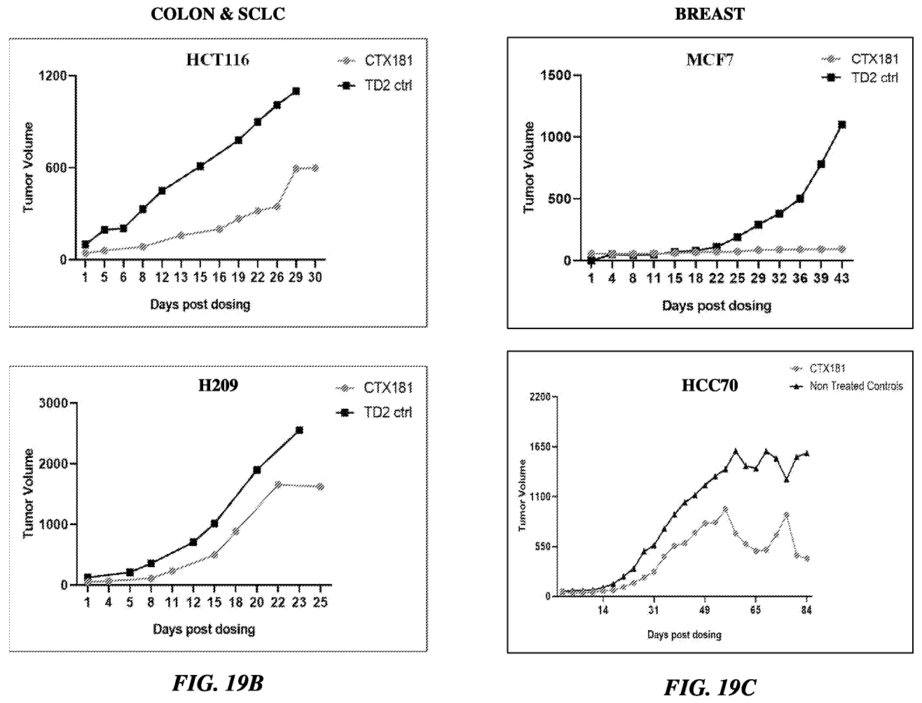

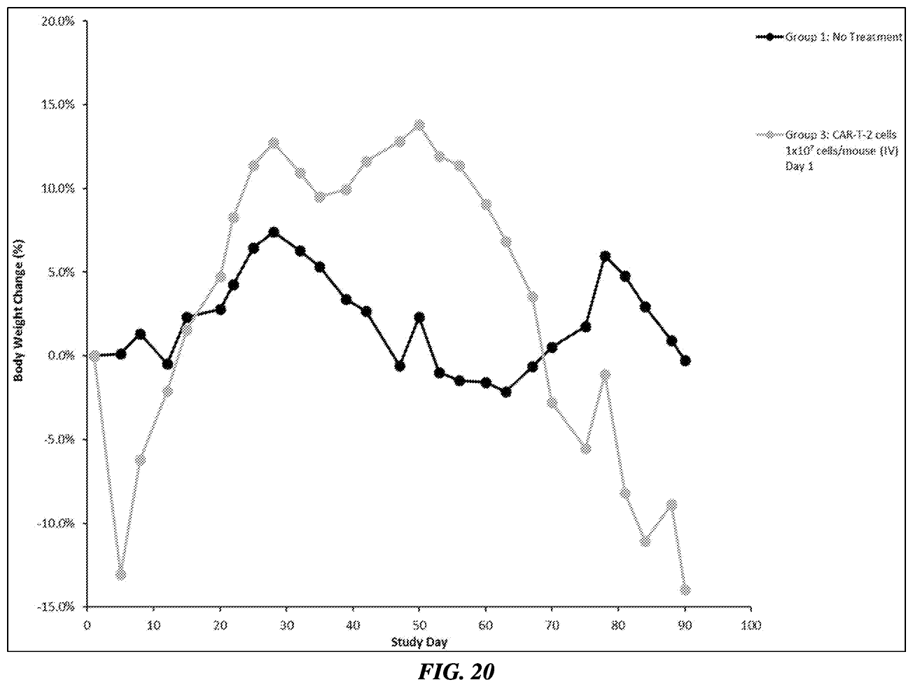

The efficacy of anti-PTK7 CAR-T cells was tested in vivo in OV90, OVCAR3, A2780 human ovarian, MCF7, HCC70 human breast, HCT116 human colon and H209 human small cell lung cancer tumor xenograft mouse models. For each xenograft model, 5 female (5-8 weeks) NOG mice were dosed single time point IV, single dose (5×10⁷ cells/ml) TRAC⁻/β2M⁻/anti-PTK7 CAR T cells (generated as previously described above). Body weight (2× weekly) and tumor volume were the endpoints measured throughout course of the study. Mice were dosed with anti-PTK7 CAR-T cells when tumors (cell lines injected subcutaneous into right flank) reached 50 mm. Studies were terminated when tumors reached endpoint size (2000 mm) or 90 days, whichever occurred first. Mice were housed and monitored under pathogen free conditions and IACUC standards. FIG. 19A shows the efficacy of anti-PTK7 CAR T cells against OV90 ovarian tumor xenograft model. FIG. 19B shows the efficacy of anti-PTK7 CAR T cells against HCT116 colon tumor xenograft model. FIG. 19C shows that anti-PTK7 CAR T cells were particularly efficacious in MCF7 tumor xenograft models. FIG. 20 shows the percent body weight change of Hs-766T pancreatic tumor xenograft model treated with PTK7 CAR T cells. Equivalent percent body weight changes were observed in all xenograft studies measured. Latent toxicity showed variability in xenograft models.

TABLE 11
CAR Components
CAR Structure:
CD8[signal peptide]-anti-Pkt7[scFV]-CD8[tm]-
CD28[co-stimulatory domain]-CD3ζ; or
CD8[signal peptide]-anti-Pkt7[scFV]-CD8[tm]-
41BB[co-stimulatory domain]-CD3ζ

| | | |
|---|---|---|
| | | SEQ |
| Name | Sequence | ID NO: |

PTK7-4

| | | |
|---|---|---|
| PKT7-4 CAR | ATGGCGCTGCCGGTGACCGCGCTGCTGCTGCCGCTGGCGCTGCTGCTGC | 49 |
| CD28 co-stim | ATGCGGCGCGCCCGCAGGTGCAGCTGGTGGAAAGCGGCGGCGGCGTGG | |
| | TGCAGCCGGGCCGCAGCCTGCGCCTGAGCTGCGCGGCGAGCGGCTTTA | |
| | CCTTTAGCAGCTATGGCATGCATTGGGTGCGCCAGGCGCCGGGCAAAGG | |
| | CCTGGAATGGGTGGCGGTGATTTGGGATGATGGCAGCAACAAATATTATG | |
| | TGGATAGCGTGAAAGGCCGCTTTACCATTAGCCGCGATAACAGCAAAAAC | |
| | ACCCTGTATCTGCAGATGAACAGCCTGCGCGCGGAAGATACCGCGGTGT | |
| | ATTATTGCGCGCGCGATGATTATTATGGCAGCGGCAGCTTTAACAGCTATT | |
| | ATGGCACCGATGTGTGGGGCCAGGGCACCACCGTGACCGTGAGCAGCG | |
| | GCGGCGGCGGCAGCGGCGGCGGCGGCAGCGGCGGCGGCGGCAGCGAA | |
| | ATTGTGCTGACCCAGAGCCCGGCGACCCTGAGCCTGAGCCCGGGCGAAC | |
| | GCGCGACCCTGAGCTGCCGCGCGAGCCAGAGCGTGAGCATTTATCTGGC | |
| | GTGGTATCAGCAGAAACCGGGCCAGGCGCCGCGCCTGCTGATTTATGAT | |
| | GCGAGCAACCGCGCGACCGGCATTCCGGCGCGCTTTAGCGGCAGCGGC | |
| | AGCGGCACCGATTTTACCCTGACCATTAGCAGCCTGGAACCGGAAGATTT | |
| | TGCGGTGTATTATTGCCAGCAGCGCAGCAACTGGCCGCCGTTTACCTTTG | |
| | GCCCGGGCACCAAAGTGGATATTAAAAGCGCGGCGGCGTTTGTGCCGGT | |
| | GTTTCTGCCGGCGAAACCGACCACCACCCCGGCGCCGCGCCCGCCGAC | |
| | CCCGGCGCCGACCATTGCGAGCCAGCCGCTGAGCCTGCGCCCGGAAGC | |
| | GTGCCGCCCGGCGGCGGGCGGCGCGGTGCATACCCGCGGCCTGGATTT | |
| | TGCGTGCGATATTTATATTTGGGCGCCGCTGGCGGGCACCTGCGGCGTG | |
| | CTGCTGCTGAGCCTGGTGATTACCCTGTATTGCAACCATCGCAACCGCAG | |
| | CAAACGCAGCCGCCTGCTGCATAGCGATTATATGAACATGACCCCGCGCC | |
| | GCCCGGGCCCGACCCGCAAACATTATCAGCCGTATGCGCCGCCGCGCGA | |
| | TTTTGCGGCGTATCGCAGCCGCGTGAAATTTAGCCGCAGCGCGGATGCG | |
| | CCGGCGTATCAGCAGGGCCAGAACCAGCTGTATAACGAACTGAACCTGG | |
| | GCCGCCGCGAAGAATATGATGTGCTGGATAAACGCCGCGGCCGCGATCC | |
| | GGAAATGGGCGGCAAACCGCGCCGCAAAAACCCGCAGGAAGGCCTGTAT | |
| | AACGAACTGCAGAAAGATAAAATGGCGGAAGCGTATAGCGAAATTGGCAT | |
| | GAAAGGCGAACGCCGCCGCGGCAAAGGCCATGATGGCCTGTATCAGGGC | |
| | CTGAGCACCGCGACCAAAGATACCTATGATGCGCTGCATATGCAGGCGCT | |
| | GCCGCCGCGC | |
| PKT7-4 CAR | ATGGCGCTTCCGGTGACAGCACTGCTCCTCCCCTTGGCGCTGTTGCTCCA | 112 |
| CD28 co-stim | CGCAGCAAGGCCGCAGGTGCAGCTGGTGGAGAGCGGCGGAGGAGTGGT | |
| | GCAACCCGGAAGGTCCCTGAGGCTCTCCTGTGCCGCCAGCGGCTTCACC | |
| | TTCTCCAGCTACGGTATGCACTGGGTGAGACAAGCCCCCGGAAAGGGCCT | |
| | CGAGTGGGTGGCCGTGATCTGGGATGATGGCTCCAACAAGTACTACGTG | |
| | GACAGCGTCAAGGGCAGATTCACCATCAGCAGGGACAACAGCAAGAACAC | |
| | CCTGTACCTGCAGATGAACTCCCTGAGAGCCGAAGACACCGCCGTGTACT | |
| | ATTGTGCCAGGGACGACTACTATGGCTCCGGCTCCTTCAATAGCTACTATG | |
| | GCACCGACGTGTGGGGCCAGGGCACCACAGTGACAGTGAGCAGCGGCG | |
| | GAGGAGGATCCGGAGGAGGAGGAAGCGGAGGAGGAGGAAGCGAGATCG | |
| | TGCTGACACAGTCCCCCGCTACCCTGAGCCTGAGCCCCGGCGAGAGAGC | |
| | TACCCTGAGCTGCAGAGCCAGCCAGAGCGTCTCCATCTACCTGGCCTGGT | |
| | ACCAGCAGAAGCCTGGCCAGGCCCCTAGACTGCTGATCTACGACGCCAG | |
| | CAACAGGGCCACCGGCATTCCTGCCAGATTCAGCGGCTCCGGCTCCGGC | |
| | ACCGATTTCACACTGACCATCAGCTCCCTGGAGCCTGAGGACTTCGCCGT | |
| | GTATTACTGCCAGCAGAGGAGCAACTGGCCCCCCTTTACCTTCGGCCCCG | |
| | GCACCAAGGTCGACATCAAGAGTGCTGCTGCCTTTGTCCCGGTATTTCTC | |
| | CCAGCCAAACCGACCACGACTCCCGCCCCGCGCCCTCCGACACCCGCTC | |
| | CCACCATCGCCTCTCAACCTCTTAGTCTTCGCCCCGAGGCATGCCGACCC | |
| | GCCGCCGGGGGTGCTGTTCATACGAGGGGCTTGGACTTCGCTTGTGATAT | |
| | TTACATTTGGGCTCCGTTGGCGGGTACGTGCGGCGTCCTTTTGTTGTCAC | |
| | TCGTTATTACTTTGTATTGTAATCACAGGAATCGCTCAAAGCGGAGTAGGT | |
| | TGTTGCATTCCGATTACATGAATATGACTCCTCGCCGGCCTGGGCCGACA | |
| | AGAAAACATTACCAACCCTATGCCCCCCCACGAGACTTCGCTGCGTACAG | |
| | GTCCCGAGTGAAGTTTTCCCGAAGCGCAGACGCTCCGGCATATCAGCAAG | |
| | GACAGAATCAGCTGTATAACGAACTGAATTTGGGACGCCGCGAGGAGTAT | |
| | GACGTGCTTGATAAACGCCGGGGGAGAGACCCGGAAATGGGGGGTAAAC | |
| | CCCGAAGAAAGAATCCCCAAGAAGGACTCTACAATGAACTCCAGAAGGAT | |
| | AAGATGGCGGAGGCCTACTCAGAAATAGGTATGAAGGGCGAACGACGAC | |
| | GGGGAAAAGGTCACGATGGCCTCTACCAAGGGTTGAGTACGGCAACCAAA | |
| | GATACGTACGATGCACTGCATATGCAGGCCCTGCCTCCCAGA | |
| PKT7-4 CAR | MALPVTALLLPLALLLHAARPQVQLVESGGGVVQPGRSLRLSCAASGFTFSS | 50 |
| CD28 co-stim | YGMHWVRQAPGKGLEWVAVIWDDGSNKYYVDSVKGRFTISRDNSKNTLYLQ | |
| | MNSLRAEDTAVYYCARDDYYGSGSFNSYYGTDVWGQGTTVTVSSGGGGSG | |
| | GGGSGGGGSEIVLTQSPATLSLSPGERATLSCRASQSVSIYLAWYQQKPGQA | |
| | PRLLIYDASNRATGIPARFSGSGSGTDFTLTISSLEPEDFAVYYCQQRSNWPP | |
| | FTFGPGTKVDIKSAAAFVPVFLPAKPTTTPAPRPPTPAPTIASQPLSLRPEACR | |
| | PAAGGAVHTRGLDFACDIYIWAPLAGTCGVLLLSLVITLYCNHRNRSKRSRLL | |
| | HSDYMNMTPRRPGPTRKHYQPYAPPRDFAAYRSRVKFSRSADAPAYQQGQ | |
| | NQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| PKT7-4b CAR | ATGGCGCTGCCGGTGACCGCGCTGCTGCTGCCGCTGGCGCTGCTGCTGC | 51 |
| 41BB co-stim | ATGCGGCGCGCCCGCAGGTGCAGCTGGTGGAAAGCGGCGGCGGCGTGG | |
| | TGCAGCCGGGCCGCAGCCTGCGCCTGAGCTGCGCGGCGAGCGGCTTTA | |
| | CCTTTAGCAGCTATGGCATGCATTGGGTGCGCCAGGCGCCGGGCAAAGG | |
| | CCTGGAATGGGTGGCGGTGATTTGGGATGATGGCAGCAACAAATATTATG | |
| | TGGATAGCGTGAAAGGCCGCTTTACCATTAGCCGCGATAACAGCAAAAAC | |
| | ACCCTGTATCTGCAGATGAACAGCCTGCGCGCGGAAGATACCGCGGTGT | |
| | ATTATTGCGCGCGCGATGATTATTATGGCAGCGGCAGCTTTAACAGCTATT | |
| | ATGGCACCGATGTGTGGGGCCAGGGCACCACCGTGACCGTGAGCAGCG | |
| | GCGGCGGCGGCAGCGGCGGCGGCGGCAGCGGCGGCGGCGGCAGCGAA | |
| | ATTGTGCTGACCCAGAGCCCGGCGACCCTGAGCCTGAGCCCGGGCGAAC | |
| | GCGCGACCCTGAGCTGCCGCGCGAGCCAGAGCGTGAGCATTTATCTGGC | |
| | GTGGTATCAGCAGAAACCGGGCCAGGCGCCGCGCCTGCTGATTTATGAT | |
| | GCGAGCAACCGCGCGACCGGCATTCCGGCGCGCTTTAGCGGCAGCGGC | |
| | AGCGGCACCGATTTTACCCTGACCATTAGCAGCCTGGAACCGGAAGATTT | |
| | TGCGGTGTATTATTGCCAGCAGCGCAGCAACTGGCCGCCGTTTACCTTTG | |
| | GCCCGGGCACCAAAGTGGATATTAAAAGCGCGGCGGCGTTTGTGCCGGT | |
| | GTTTCTGCCGGCGAAACCGACCACCACCCCGGCGCCGCGCCCGCCGAC | |
| | CCCGGCGCCGACCATTGCGAGCCAGCCGCTGAGCCTGCGCCCGGAAGC | |
| | GTGCCGCCCGGCGGCGGGCGGCGCGGTGCATACCCGCGGCCTGGATTT | |
| | TGCGTGCGATATTTATATTTGGGCGCCGCTGGCGGGCACCTGCGGCGTG | |
| | CTGCTGCTGAGCCTGGTGATTACCCTGTATTGCAACCATCGCAACCGCAA | |
| | ACGCGGCCGCAAAAAACTGCTGTATATTTTTAAACAGCCGTTTATGCGCCC | |
| | GGTGCAGACCACCCAGGAAGAAGATGGCTGCAGCTGCCGCTTTCCGGAA | |
| | GAAGAAGAAGGCGGCTGCGAACTGCGCGTGAAATTTAGCCGCAGCGCGG | |
| | ATGCGCCGGCGTATCAGCAGGGCCAGAACCAGCTGTATAACGAACTGAA | |
| | CCTGGGCCGCCGCGAAGAATATGATGTGCTGGATAAACGCCGCGGCCGC | |
| | GATCCGGAAATGGGCGGCAAACCGCGCCGCAAAAACCCGCAGGAAGGC | |
| | CTGTATAACGAACTGCAGAAAGATAAAATGGCGGAAGCGTATAGCGAAAT | |
| | TGGCATGAAAGGCGAACGCCGCCGCGGCAAAGGCCATGATGGCCTGTAT | |
| | CAGGGCCTGAGCACCGCGACCAAAGATACCTATGATGCGCTGCATATGCA | |
| | GGCGCTGCCGCCGCGC | |
| PKT7-4b CAR | MALPVTALLLPLALLLHAARPQVQLVESGGGVVQPGRSLRLSCAASGFTFSS | 52 |
| 41BB co-stim | YGMHWVRQAPGKGLEWVAVIWDDGSNKYYVDSVKGRFTISRDNSKNTLYLQ | |
| | MNSLRAEDTAVYYCARDDYYGSGSFNSYYGTDVWGQGTTVTVSSGGGGSG | |
| | GGGSGGGGSEIVLTQSPATLSLSPGERATLSCRASQSVSIYLAWYQQKPGQA | |
| | PRLLIYDASNRATGIPARFSGSGSGTDFTLTISSLEPEDFAVYYCQQRSNWPP | |
| | FTFGPGTKVDIKSAAAFVPVFLPAKPTTTPAPRPPTPAPTIASQPLSLRPEACR | |
| | PAAGGAVHTRGLDFACDIYIWAPLAGTCGVLLLSLVITLYCNHRNRKRGRKKL | |
| | LYIFKQPFMRPVQTTQEEDGCSCRFPEEEEGGCELRVKFSRSADAPAYQQG | |
| | QNQLYNELNLGRREEYDVLDKRRGRDPEMGGKPRRKNPQEGLYNELQKDK | |
| | MAEAYSEIGMKGERRRGKGHDGLYQGLSTATKDTYDALHMQALPPR | |
| PTK7-4 scFv | CAGGTGCAGCTGGTGGAAAGCGGCGGCGGCGTGGTGCAGCCGGGCCGC | 53 |
| | AGCCTGCGCCTGAGCTGCGCGGCGAGCGGCTTTACCTTTAGCAGCTATG | |
| | GCATGCATTGGGTGCGCCAGGCGCCGGGCAAAGGCCTGGAATGGGTGG | |
| | CGGTGATTTGGGATGATGGCAGCAACAAATATTATGTGGATAGCGTGAAA | |
| | GGCCGCTTTACCATTAGCCGCGATAACAGCAAAAACACCCTGTATCTGCA | |
| | GATGAACAGCCTGCGCGCGGAAGATACCGCGGTGTATTATTGCGCGCGC | |
| | GATGATTATTATGGCAGCGGCAGCTTTAACAGCTATTATGGCACCGATGTG | |
| | TGGGGCCAGGGCACCACCGTGACCGTGAGCAGCGGCGGCGGCGGCAGC | |
| | GGCGGCGGCGGCAGCGGCGGCGGCGGCAGCGAAATTGTGCTGACCCAG | |
| | AGCCCGGCGACCCTGAGCCTGAGCCCGGGCGAACGCGCGACCCTGAGC | |
| | TGCCGCGCGAGCCAGAGCGTGAGCATTTATCTGGCGTGGTATCAGCAGA | |
| | AACCGGGCCAGGCGCCGCGCCTGCTGATTTATGATGCGAGCAACCGCGC | |
| | GACCGGCATTCCGGCGCGCTTTAGCGGCAGCGGCAGCGGCACCGATTTT | |
| | ACCCTGACCATTAGCAGCCTGGAACCGGAAGATTTTGCGGTGTATTATTG | |
| | CCAGCAGCGCAGCAACTGGCCGCCGTTTACCTTTGGCCCGGGCACCAAA | |
| | GTGGATATTAAA | |
| PTK7-4 scFv | CAGGTGCAGCTGGTGGAAAGCGGCGGCGGCGTGGTGCAGCCGGGCCGC | 113 |
| | AGCCTGCGCCTGAGCTGCGCGGCGAGCGGCTTTACCTTTAGCAGCTATG | |
| | GCATGCATTGGGTGCGCCAGGCGCCGGGCAAAGGCCTGGAATGGGTGG | |
| | CGGTGATTTGGGATGATGGCAGCAACAAATATTATGTGGATAGCGTGAAA | |
| | GGCCGCTTTACCATTAGCCGCGATAACAGCAAAAACACCCTGTATCTGCA | |
| | GATGAACAGCCTGCGCGCGGAAGATACCGCGGTGTATTATTGCGCGCGC | |
| | GATGATTATTATGGCAGCGGCAGCTTTAACAGCTATTATGGCACCGATGTG | |
| | TGGGGCCAGGGCACCACCGTGACCGTGAGCAGCGGCGGCGGCGGCAGC | |
| | GGCGGCGGCGGCAGCGGCGGCGGCGGCAGCGAAATTGTGCTGACCCAG | |
| | AGCCCGGCGACCCTGAGCCTGAGCCCGGGCGAACGCGCGACCCTGAGC | |
| | TGCCGCGCGAGCCAGAGCGTGAGCATTTATCTGGCGTGGTATCAGCAGAA | |
| | ACCGGGCCAGGCGCCGCGCCTGCTGATTTATGATGCGAGCAACCGCGCG | |
| | ACCGGCATTCCGGCGCGCTTTAGCGGCAGCGGCAGCGGCACCGATTTTA | |
| | CCCTGACCATTAGCAGCCTGGAACCGGAAGATTTTGCGGTGTATTATTGC | |
| | CAGCAGCGCAGCAACTGGCCGCCGTTTACCTTTGGCCCGGGCACCAAAG | |
| | TGGATATTAAA | |
| PTK7-4 scFv | QVQLVESGGGVVQPGRSLRLSCAASGFTFSSYGMHWVRQAPGKGLEWVAV | 54 |
| (linker | IWDDGSNKYYVDSVKGRFTISRDNSKNTLYLQMNSLRAEDTAVYYCARDDYY | |
| underlined) | GSGSFNSYYGTDVWGQGTTVTVSSGGGGSGGGGSGGGGSEIVLTQSPATL | |
| | SLSPGERATLSCRASQSVSIYLAWYQQKPGQAPRLLIYDASNRATGIPARFSG | |
| | SGSGTDFTLTISSLEPEDFAVYYCQQRSNWPPFTFGPGTKVDIK | |
| PTK7-4 scFv VH | QVQLVESGGGVVQPGRSLRLSCAASGFTFSSYGMHWVRQAPGKGLEWVAV | 55 |
| CDRs- in bold | IWDDGSNKYYVDSVKGRFTISRDNSKNTLYLQMNSLRAEDTAVYYCARDDYY | |
| | GSGSFNSYYGTDVWGQGTTVTVSS | |
| PTK7-4 scFv VL | EIVLTQSPATLSLSPGERATLSCRASQSVSIYLAWYQQKPGQAPRLLIYDASN | 56 |
| CDRs- in bold | RATGIPARFSGSGSGTDFTLTISSLEPEDFAVYYCQQRSNWPPFTFGPGTKV | |
| | DIK | |
| PTK7-4 | SYGMH | 57 |
| VH CDR1 | | |
| PTK7-4 | VIWDDGSNKYYVDSVKG | 58 |
| VH CDR2 | | |
| PTK7-4 | DDYYGSGSFNSYYGTDV | 59 |
| VH CDR3 | | |
| PTK7-4 | RASQSVSIYLA | 60 |
| VL CDR1 | | |
| PTK7-4 | DASNRAT | 61 |
| VL CDR2 | | |
| PTK7-4 | QQRSNWPPFT | 62 |
| VL CDR3 | | |
| PTK7-4 | GAGATGTAAGGAGCTGCTGTGACTTGCTCAAGGCCTTATATCGAGTAAAC | 63 |
| Donor | GGTAGTGCTGGGGCTTAGACGCAGGTGTTCTGATTTATAGTTCAAAACCT | |
| LHA to RHA | CTATCAATGAGAGAGCAATCTCCTGGTAATGTGATAGATTTCCCAACTTAA | |
| | TGCCAACATACCATAAACCTCCCATTCTGCTAATGCCCAGCCTAAGTTGGG | |
| | GAGACCACTCCAGATTCCAAGATGTACAGTTTGCTTTGCTGGGCCTTTTTC | |
| | CCATGCCTGCCTTTACTCTGCCAGAGTTATATTGCTGGGGTTTTGAAGAAG | |
| | ATCCTATTAAATAAAAGAATAAGCAGTATTATTAAGTAGCCCTGCATTTCAG | |
| | GTTTCCTTGAGTGGCAGGCCAGGCCTGGCCGTGAACGTTCACTGAAATCA | |
| | TGGCCTCTTGGCCAAGATTGATAGCTTGTGCCTGTCCCTGAGTCCCAGTC | |
| | CATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAA | |
| | CCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCG | |
| | ATTTTGATTCTCAAACAAATGTGTCACAAAGTAAGGATTCTGATGTGTATAT | |
| | CACAGACAAAACTGTGCTAGACATGAGGTCTATGGACTTCAGGCTCCGGT | |
| | GCCCGTCAGTGGGCAGAGCGCACATCGCCCACAGTCCCCGAGAAGTTGG | |
| | GGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCG | |
| | CAACGGGTTTGCCGCCAGAACACAGGTAAGTGCCGTGTGTGGTTCCCGC | |
| | GGGCCTGGCCTCTTTACGGGTTATGGCCCTTGCGTGCCTTGAATTACTTC | |
| | CACTGGCTGCAGTACGTGATTCTTGATCCCGAGCTTCGGGTTGGAAGTGG | |
| | GTGGGAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAATCTGG | |
| | TGGCACCTTCGCGCCTGTCTCGCTGCTTTCGATAAGTCTCTAGCCATTTAA | |
| | AATTTTTGATGACCTGCTGCGACGCTTTTTTTCTGGCAAGATAGTCTTGTAA | |
| | ATGCGGGCCAAGATCTGCACACTGGTATTTCGGTTTTTGGGGCCGCGGG | |
| | CGGCGACGGGGCCCGTGCGTCCCAGCGCACATGTTCGGCGAGGCGGGG | |
| | CCTGCGAGCGCGGCCACCGAGAATCGGACGGGGGTAGTCTCAAGCTGG | |
| | CCGGCCTGCTCTGGTGCCTGGCCTCGCGCCGCCGTGTATCGCCCCGCCC | |
| | TGGGCGGCAAGGCTGGCCCGGTCGGCACCAGTTGCGTGAGCGGAAAGA | |
| | TGGCCGCTTCCCGGCCCTGCTGCAGGGAGCTCAAAATGGAGGACGCGGC | |
| | GCTCGGGAGAGCGGGCGGGTGAGTCACCCACACAAAGGAAAAGGGCCTT | |
| | TCCGTCCTCAGCCGTCGCTTCATGTGACTCCACGGAGTACCGGGCGCCG | |
| | TCCAGGCACCTCGATTAGTTCTCGAGCTTTTGGAGTACGTCGTCTTTAGGT | |
| | TGGGGGGAGGGGTTTTATGCGATGGAGTTTCCCCACACTGAGTGGGTGG | |
| | AGACTGAAGTTAGGCCAGCTTGGCACTTGATGTAATTCTCCTTGGAATTTG | |
| | CCCTTTTTGAGTTTGGATCTTGGTTCATTCTCAAGCCTCAGACAGTGGTTC | |
| | AAAGTTTTTTTCTTCCATTTCAGGTGTCGTGACCACCATGGCGCTTCCGGT | |
| | GACAGCACTGCTCCTCCCCTTGGCGCTGTTGCTCCACGCAGCAAGGCCG | |
| | CAGGTGCAGCTGGTGGAGAGCGGCGGAGGAGTGGTGCAACCCGGAAGG | |
| | TCCCTGAGGCTCTCCTGTGCCGCCAGCGGCTTCACCTTCTCCAGCTACGG | |
| | TATGCACTGGGTGAGACAAGCCCCCGGAAAGGGCCTCGAGTGGGTGGCC | |
| | GTGATCTGGGATGATGGCTCCAACAAGTACTACGTGGACAGCGTCAAGGG | |
| | CAGATTCACCATCAGCAGGGACAACAGCAAGAACACCCTGTACCTGCAGA | |
| | TGAACTCCCTGAGAGCCGAAGACACCGCCGTGTACTATTGTGCCAGGGAC | |
| | GACTACTATGGCTCCGGCTCCTTCAATAGCTACTATGGCACCGACGTGTG | |
| | GGGCCAGGGCACCACAGTGACAGTGAGCAGCGGCGGAGGAGGATCCGG | |
| | AGGAGGAGGAAGCGGAGGAGGAGGAAGCGAGATCGTGCTGACACAGTC | |
| | CCCCGCTACCCTGAGCCTGAGCCCCGGCGAGAGAGCTACCCTGAGCTGC | |
| | AGAGCCAGCCAGAGCGTCTCCATCTACCTGGCCTGGTACCAGCAGAAGC | |
| | CTGGCCAGGCCCCTAGACTGCTGATCTACGACGCCAGCAACAGGGCCAC | |
| | CGGCATTCCTGCCAGATTCAGCGGCTCCGGCTCCGGCACCGATTTCACAC | |
| | TGACCATCAGCTCCCTGGAGCCTGAGGACTTCGCCGTGTATTACTGCCAG | |
| | CAGAGGAGCAACTGGCCCCCCTTTACCTTCGGCCCCGGCACCAAGGTCG | |
| | ACATCAAGAGTGCTGCTGCCTTTGTCCCGGTATTTCTCCCAGCCAAACCG | |
| | ACCACGACTCCCGCCCCGCGCCCTCCGACACCCGCTCCCACCATCGCCT | |
| | CTCAACCTCTTAGTCTTCGCCCCGAGGCATGCCGACCCGCCGCCGGGGG | |
| | TGCTGTTCATACGAGGGGCTTGGACTTCGCTTGTGATATTTACATTTGGGC | |
| | TCCGTTGGCGGGTACGTGCGGCGTCCTTTTGTTGTCACTCGTTATTACTTT | |
| | GTATTGTAATCACAGGAATCGCTCAAAGCGGAGTAGGTTGTTGCATTCCG | |
| | ATTACATGAATATGACTCCTCGCCGGCCTGGGCCGACAAGAAAACATTAC | |
| | CAACCCTATGCCCCCCCACGAGACTTCGCTGCGTACAGGTCCCGAGTGAA | |
| | GTTTTCCCGAAGCGCAGACGCTCCGGCATATCAGCAAGGACAGAATCAGC | |
| | TGTATAACGAACTGAATTTGGGACGCCGCGAGGAGTATGACGTGCTTGAT | |
| | AAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGA | |
| | GGCCTACTCAGAAATAGGTATGAAGGGCGAACGACGACGGGGAAAAGGT | |
| | CACGATGGCCTCTACCAAGGGTTGAGTACGGCAACCAAAGATACGTACGA | |
| | TGCACTGCATATGCAGGCCCTGCCTCCCAGATAATAATAAAATCGCTATCC | |
| | ATCGAAGATGGATGTGTGTTGGTTTTTTGTGTGTGGAGCAACAAATCTGAC | |
| | TTTGCATGTGCAAACGCCTTCAACAACAGCATTATTCCAGAAGACACCTTC | |
| | TTCCCCAGCCCAGGTAAGGGCAGCTTTGGTGCCTTCGCAGGCTGTTTCCT | |
| | TGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTC | |
| | TTTTTACTAAGAAACAGTGAGCCTTGTTCTGGCAGTCCAGAGAATGACACG | |
| | GGAAAAAAGCAGATGAAGAGAAGGTGGCAGGAGAGGGCACGTGGCCCA | |
| | GCCTCAGTCTCTCCAACTGAGTTCCTGCCTGCCTGCCTTTGCTCAGACTG | |
| | TTTGCCCCTTACTGCTCTTCTAGGCCTCATTCTAAGCCCCTTCTCCAAGTT | |
| | GCCTCTCCTTATTTCTCCCTGTCTGCCAAAAAATCTTTCCCAGCTCACTAA | |
| | GTCAGTCTCACGCAGTCACTCATTAACCCACCAATCACTGATTGTGCCGG | |
| | CACATGAATGCACCAGGTGTTGAAGTGGAGGAATTAAAAAGTCAGATGAG | |
| | GGGTGTGCCCAGAGGAAGCACCATTCTAGTTGGGGGAGCCCATCTGTCA | |
| | GCTGGGAAAAGTCCAAATAACTTCAGATTGGAATGTGTTTTAACTCAGGGT | |
| | TGAGAAAACAGCTACCTTCAGGACAAAAGTCAGGGAAGGGCTCTCTGAAG | |
| | AAATGCTACTTGAAGATACCAGCCCTACCAAGGGCAGGGAGAGGACCCTA | |
| | TAGAGGCCTGGGACAGGAGCTCAATGAGAAAGG | |
| PTK7-4b | GAGATGTAAGGAGCTGCTGTGACTTGCTCAAGGCCTTATATCGAGTAAAC | 64 |
| Donor | GGTAGTGCTGGGGCTTAGACGCAGGTGTTCTGATTTATAGTTCAAAACCT | |
| LHA to RHA | CTATCAATGAGAGAGCAATCTCCTGGTAATGTGATAGATTTCCCAACTTAA | |
| | TGCCAACATACCATAAACCTCCCATTCTGCTAATGCCCAGCCTAAGTTGGG | |
| | GAGACCACTCCAGATTCCAAGATGTACAGTTTGCTTTGCTGGGCCTTTTTC | |
| | CCATGCCTGCCTTTACTCTGCCAGAGTTATATTGCTGGGGTTTTGAAGAAG | |
| | ATCCTATTAAATAAAAGAATAAGCAGTATTATTAAGTAGCCCTGCATTTCAG | |
| | GTTTCCTTGAGTGGCAGGCCAGGCCTGGCCGTGAACGTTCACTGAAATCA | |
| | TGGCCTCTTGGCCAAGATTGATAGCTTGTGCCTGTCCCTGAGTCCCAGTC | |
| | CATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAA | |
| | CCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCG | |
| | ATTTTGATTCTCAAACAAATGTGTCACAAAGTAAGGATTCTGATGTGTATAT | |
| | CACAGACAAAACTGTGCTAGACATGAGGTCTATGGACTTCAGGCTCCGGT | |
| | GCCCGTCAGTGGGCAGAGCGCACATCGCCCACAGTCCCCGAGAAGTTGG | |
| | GGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCG | |
| | CAACGGGTTTGCCGCCAGAACACAGGTAAGTGCCGTGTGTGGTTCCCGC | |
| | GGGCCTGGCCTCTTTACGGGTTATGGCCCTTGCGTGCCTTGAATTACTTC | |
| | CACTGGCTGCAGTACGTGATTCTTGATCCCGAGCTTCGGGTTGGAAGTGG | |
| | GTGGGAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAATCTGG | |
| | TGGCACCTTCGCGCCTGTCTCGCTGCTTTCGATAAGTCTCTAGCCATTTAA | |
| | AATTTTTGATGACCTGCTGCGACGCTTTTTTTCTGGCAAGATAGTCTTGTAA | |
| | ATGCGGGCCAAGATCTGCACACTGGTATTTCGGTTTTTGGGGCCGCGGG | |
| | CGGCGACGGGGCCCGTGCGTCCCAGCGCACATGTTCGGCGAGGCGGGG | |
| | CCTGCGAGCGCGGCCACCGAGAATCGGACGGGGGTAGTCTCAAGCTGG | |
| | CCGGCCTGCTCTGGTGCCTGGCCTCGCGCCGCCGTGTATCGCCCCGCCC | |
| | TGGGCGGCAAGGCTGGCCCGGTCGGCACCAGTTGCGTGAGCGGAAAGA | |
| | TGGCCGCTTCCCGGCCCTGCTGCAGGGAGCTCAAAATGGAGGACGCGGC | |
| | GCTCGGGAGAGCGGGCGGGTGAGTCACCCACACAAAGGAAAAGGGCCTT | |
| | TCCGTCCTCAGCCGTCGCTTCATGTGACTCCACGGAGTACCGGGCGCCG | |
| | TCCAGGCACCTCGATTAGTTCTCGAGCTTTTGGAGTACGTCGTCTTTAGGT | |
| | TGGGGGGAGGGGTTTTATGCGATGGAGTTTCCCCACACTGAGTGGGTGG | |
| | AGACTGAAGTTAGGCCAGCTTGGCACTTGATGTAATTCTCCTTGGAATTTG | |
| | CCCTTTTTGAGTTTGGATCTTGGTTCATTCTCAAGCCTCAGACAGTGGTTC | |
| | AAAGTTTTTTTCTTCCATTTCAGGTGTCGTGACCACCATGGCGCTTCCGGT | |
| | GACAGCACTGCTCCTCCCCTTGGCGCTGTTGCTCCACGCAGCAAGGCCG | |
| | CAGGTGCAGCTGGTGGAGAGCGGCGGAGGAGTGGTGCAACCCGGAAGG | |
| | TCCCTGAGGCTCTCCTGTGCCGCCAGCGGCTTCACCTTCTCCAGCTACGG | |
| | TATGCACTGGGTGAGACAAGCCCCCGGAAAGGGCCTCGAGTGGGTGGCC | |
| | GTGATCTGGGATGATGGCTCCAACAAGTACTACGTGGACAGCGTCAAGGG | |
| | CAGATTCACCATCAGCAGGGACAACAGCAAGAACACCCTGTACCTGCAGA | |
| | TGAACTCCCTGAGAGCCGAAGACACCGCCGTGTACTATTGTGCCAGGGAC | |
| | GACTACTATGGCTCCGGCTCCTTCAATAGCTACTATGGCACCGACGTGTG | |
| | GGGCCAGGGCACCACAGTGACAGTGAGCAGCGGCGGAGGAGGATCCGG | |
| | AGGAGGAGGAAGCGGAGGAGGAGGAAGCGAGATCGTGCTGACACAGTC | |
| | CCCCGCTACCCTGAGCCTGAGCCCCGGCGAGAGAGCTACCCTGAGCTGC | |
| | AGAGCCAGCCAGAGCGTCTCCATCTACCTGGCCTGGTACCAGCAGAAGC | |
| | CTGGCCAGGCCCCTAGACTGCTGATCTACGACGCCAGCAACAGGGCCAC | |
| | CGGCATTCCTGCCAGATTCAGCGGCTCCGGCTCCGGCACCGATTTCACAC | |
| | TGACCATCAGCTCCCTGGAGCCTGAGGACTTCGCCGTGTATTACTGCCAG | |
| | CAGAGGAGCAACTGGCCCCCCTTTACCTTCGGCCCCGGCACCAAGGTCG | |
| | ACATCAAGAGTGCTGCTGCCTTTGTCCCGGTATTTCTCCCAGCCAAACCG | |
| | ACCACGACTCCCGCCCCGCGCCCTCCGACACCCGCTCCCACCATCGCCT | |
| | CTCAACCTCTTAGTCTTCGCCCCGAGGCATGCCGACCCGCCGCCGGGGG | |
| | TGCTGTTCATACGAGGGGCTTGGACTTCGCTTGTGATATTTACATTTGGGC | |
| | TCCGTTGGCGGGTACGTGCGGCGTCCTTTTGTTGTCACTCGTTATTACTTT | |
| | GTATTGTAATCACAGGAATCGCAAACGGGGCAGAAAGAAACTCCTGTATA | |
| | TATTCAAACAACCATTTATGAGACCAGTACAAACTACTCAAGAGGAAGATG | |
| | GCTGTAGCTGCCGATTTCCAGAAGAAGAAGAAGGAGGATGTGAACTGCGA | |
| | GTGAAGTTTTCCCGAAGCGCAGACGCTCCGGCATATCAGCAAGGACAGAA | |
| | TCAGCTGTATAACGAACTGAATTTGGGACGCCGCGAGGAGTATGACGTGC | |
| | TTGATAAACGCCGGGGGAGAGACCCGGAAATGGGGGGTAAACCCCGAAG | |
| | AAAGAATCCCCAAGAAGGACTCTACAATGAACTCCAGAAGGATAAGATGG | |
| | CGGAGGCCTACTCAGAAATAGGTATGAAGGGCGAACGACGACGGGGAAA | |
| | AGGTCACGATGGCCTCTACCAAGGGTTGAGTACGGCAACCAAAGATACGT | |
| | ACGATGCACTGCATATGCAGGCCCTGCCTCCCAGATAATAATAAAATCGCT | |
| | ATCCATCGAAGATGGATGTGTGTTGGTTTTTTGTGTGTGGAGCAACAAATC | |
| | TGACTTTGCATGTGCAAACGCCTTCAACAACAGCATTATTCCAGAAGACAC | |
| | CTTCTTCCCCAGCCCAGGTAAGGGCAGCTTTGGTGCCTTCGCAGGCTGTT | |
| | TCCTTGCTTCAGGAATGGCCAGGTTCTGCCCAGAGCTCTGGTCAATGATG | |
| | TCTAAAACTCCTCTGATTGGTGGTCTCGGCCTTATCCATTGCCACCAAAAC | |
| | CCTCTTTTTACTAAGAAACAGTGAGCCTTGTTCTGGCAGTCCAGAGAATGA | |
| | CACGGGAAAAAAGCAGATGAAGAGAAGGTGGCAGGAGAGGGCACGTGG | |
| | CCCAGCCTCAGTCTCTCCAACTGAGTTCCTGCCTGCCTGCCTTTGCTCAG | |
| | ACTGTTTGCCCCTTACTGCTCTTCTAGGCCTCATTCTAAGCCCCTTCTCCA | |
| | AGTTGCCTCTCCTTATTTCTCCCTGTCTGCCAAAAAATCTTTCCCAGCTCA | |
| | CTAAGTCAGTCTCACGCAGTCACTCATTAACCCACCAATCACTGATTGTGC | |
| | CGGCACATGAATGCACCAGGTGTTGAAGTGGAGGAATTAAAAAGTCAGAT | |
| | GAGGGGTGTGCCCAGAGGAAGCACCATTCTAGTTGGGGGAGCCCATCTG | |
| | TCAGCTGGGAAAAGTCCAAATAACTTCAGATTGGAATGTGTTTTAACTCAG | |
| | GGTTGAGAAAACAGCTACCTTCAGGACAAAAGTCAGGGAAGGGCTCTCTG | |
| | AAGAAATGCTACTTGAAGATACCAGCCCTACCAAGGGCAGGGAGAGGAC | |
| | CCTATAGAGGCCTGGGACAGGAGCTCAATGAGAAAGG | |

PTK7-7

| | | |
|---|---|---|
| PKT7-7 CAR | ATGGCGCTGCCGGTGACCGCGCTGCTGCTGCCGCTGGCGCTGCTGCTGC | 65 |
| | ATGCGGCGCGCCCGGAAATTGTGCTGACCCAGAGCCCGGCGACCCTGAG | |
| | CCTGAGCCCGGGCGAACGCGCGACCCTGAGCTGCCGCGCGAGCCAGAG | |
| | CGTGAGCATTTATCTGGCGTGGTATCAGCAGAAACCGGGCCAGGCGCCG | |
| | CGCCTGCTGATTTATGATGCGAGCAACCGCGCGACCGGCATTCCGGCGC | |
| | GCTTTAGCGGCAGCGGCAGCGGCACCGATTTTACCCTGACCATTAGCAGC | |
| | CTGGAACCGGAAGATTTTGCGGTGTATTATTGCCAGCAGCGCAGCAACTG | |
| | GCCGCCGTTTACCTTTGGCCCGGGCACCAAAGTGGATATTAAAGGCGGC | |
| | GGCGGCAGCGGCGGCGGCGGCAGCGGCGGCGGCGGCAGCCAGGTGCA | |
| | GCTGGTGGAAAGCGGCGGCGGCGTGGTGCAGCCGGGCCGCAGCCTGCG | |
| | CCTGAGCTGCGCGGCGAGCGGCTTTACCTTTAGCAGCTATGGCATGCATT | |
| | GGGTGCGCCAGGCGCCGGGCAAAGGCCTGGAATGGGTGGCGGTGATTT | |
| | GGGATGATGGCAGCAACAAATATTATGTGGATAGCGTGAAAGGCCGCTTT | |
| | ACCATTAGCCGCGATAACAGCAAAAACACCCTGTATCTGCAGATGAACAG | |
| | CCTGCGCGCGGAAGATACCGCGGTGTATTATTGCGCGCGCGATGATTATT | |
| | ATGGCAGCGGCAGCTTTAACAGCTATTATGGCACCGATGTGTGGGGCCAG | |
| | GGCACCACCGTGACCGTGAGCAGCGCGGCGGCGTTTGTGCCGGTGTTTC | |
| | TGCCGGCGAAACCGACCACCACCCCGGCGCCGCGCCCGCCGACCCCGG | |
| | CGCCGACCATTGCGAGCCAGCCGCTGAGCCTGCGCCCGGAAGCGTGCC | |
| | GCCCGGCGGCGGGCGGCGCGGTGCATACCCGCGGCCTGGATTTTGCGT | |
| | GCGATATTTATATTTGGGCGCCGCTGGCGGGCACCTGCGGCGTGCTGCT | |
| | GCTGAGCCTGGTGATTACCCTGTATTGCAACCATCGCAACCGCAGCAAAC | |
| | GCAGCCGCCTGCTGCATAGCGATTATATGAACATGACCCCGCGCCGCCC | |
| | GGGCCCGACCCGCAAACATTATCAGCCGTATGCGCCGCCGCGCGATTTT | |
| | GCGGCGTATCGCAGCCGCGTGAAATTTAGCCGCAGCGCGGATGCGCCGG | |
| | CGTATCAGCAGGGCCAGAACCAGCTGTATAACGAACTGAACCTGGGCCG | |
| | CCGCGAAGAATATGATGTGCTGGATAAACGCCGCGGCCGCGATCCGGAA | |
| | ATGGGCGGCAAACCGCGCCGCAAAAACCCGCAGGAAGGCCTGTATAACG | |
| | AACTGCAGAAAGATAAAATGGCGGAAGCGTATAGCGAAATTGGCATGAAA | |
| | GGCGAACGCCGCCGCGGCAAAGGCCATGATGGCCTGTATCAGGGCCTGA | |
| | GCACCGCGACCAAAGATACCTATGATGCGCTGCATATGCAGGCGCTGCC | |
| | GCCGCGC | |
| PKT7-7 CAR | MALPVTALLLPLALLLHAARPEIVLTQSPATLSLSPGERATLSCRASQSVSIYLA | 66 |
| | WYQQKPGQAPRLLIYDASNRATGIPARFSGSGSGTDFTLTISSLEPEDFAVYY | |
| | CQQRSNWPPFTFGPGTKVDIKGGGGSGGGGSGGGGSQVQLVESGGGVVQ | |
| | PGRSLRLSCAASGFTFSSYGMHWVRQAPGKGLEWVAVIWDDGSNKYYVDS | |
| | VKGRFTISRDNSKNTLYLQMNSLRAEDTAVYYCARDDYYGSGSFNSYYGTDV | |
| | WGQGTTVTVSSAAAFVPVFLPAKPTTTPAPRPPTPAPTIASQPLSLRPEACRP | |
| | AAGGAVHTRGLDFACDIYIWAPLAGTCGVLLLSLVITLYCNHRNRSKRSRLLH | |
| | SDYMNMTPRRPGPTRKHYQPYAPPRDFAAYRSRVKFSRSADAPAYQQGQN | |
| | QLYNELNLGRREEYDVLDKRRGRDPEMGGKPRRKNPQEGLYNELQKDKMA | |
| | EAYSEIGMKGERRRGKGHDGLYQGLSTATKDTYDALHMQALPPR | |
| PTK7-7 scFv | GAAATTGTGCTGACCCAGAGCCCGGCGACCCTGAGCCTGAGCCCGGGCG | 67 |
| | AACGCGCGACCCTGAGCTGCCGCGCGAGCCAGAGCGTGAGCATTTATCT | |
| | GGCGTGGTATCAGCAGAAACCGGGCCAGGCGCCGCGCCTGCTGATTTAT | |
| | GATGCGAGCAACCGCGCGACCGGCATTCCGGCGCGCTTTAGCGGCAGC | |
| | GGCAGCGGCACCGATTTTACCCTGACCATTAGCAGCCTGGAACCGGAAG | |
| | ATTTTGCGGTGTATTATTGCCAGCAGCGCAGCAACTGGCCGCCGTTTACC | |
| | TTTGGCCCGGGCACCAAAGTGGATATTAAAGGCGGCGGCGGCAGCGGCG | |
| | GCGGCGGCAGCGGCGGCGGCGGCAGCCAGGTGCAGCTGGTGGAAAGC | |
| | GGCGGCGGCGTGGTGCAGCCGGGCCGCAGCCTGCGCCTGAGCTGCGCG | |
| | GCGAGCGGCTTTACCTTTAGCAGCTATGGCATGCATTGGGTGCGCCAGG | |
| | CGCCGGGCAAAGGCCTGGAATGGGTGGCGGTGATTTGGGATGATGGCAG | |
| | CAACAAATATTATGTGGATAGCGTGAAAGGCCGCTTTACCATTAGCCGCG | |
| | ATAACAGCAAAAACACCCTGTATCTGCAGATGAACAGCCTGCGCGCGGAA | |
| | GATACCGCGGTGTATTATTGCGCGCGCGATGATTATTATGGCAGCGGCAG | |
| | CTTTAACAGCTATTATGGCACCGATGTGTGGGGCCAGGGCACCACCGTGA | |
| | CCGTGAGCAGC | |
| PTK7-7 scFv | EIVLTQSPATLSLSPGERATLSCRASQSVSIYLAWYQQKPGQAPRLLIYDASN | 68 |
| (linker | RATGIPARFSGSGSGTDFTLTISSLEPEDFAVYYCQQRSNWPPFTFGPGTKV | |
| underlined) | DIKGGGGSGGGGSGGGGSQVQLVESGGGVVQPGRSLRLSCAASGFTFSSY | |
| | GMHWVRQAPGKGLEWVAVIWDDGSNKYYVDSVKGRFTISRDNSKNTLYLQ | |
| | MNSLRAEDTAVYYCARDDYYGSGSFNSYYGTDVWGQGTTVTVSS | |
| PTK7-7 scFv | QVQLVESGGGVVQPGRSLRLSCAASGFTFSSYGMHWVRQAPGKGLEWVAV | 69 |
| VH | IWDDGSNKYYVDSVKGRFTISRDNSKNTLYLQMNSLRAEDTAVYYCARDDYY | |
| | GSGSFNSYYGTDVWGQGTTVTVSS | |
| PTK7-7 scFv | EIVLTQSPATLSLSPGERATLSCRASQSVSIYLAWYQQKPGQAPRLLIYDASN | 70 |
| VL | RATGIPARFSGSGSGTDFTLTISSLEPEDFAVYYCQQRSNWPPFTFGPGTKV | |
| | DIK | |
| PTK7-7 | GAGATGTAAGGAGCTGCTGTGACTTGCTCAAGGCCTTATATCGAGTAAAC | 71 |
| Donor | GGTAGTGCTGGGGCTTAGACGCAGGTGTTCTGATTTATAGTTCAAAACCT | |
| LHA to RHA | CTATCAATGAGAGAGCAATCTCCTGGTAATGTGATAGATTTCCCAACTTAA | |
| | TGCCAACATACCATAAACCTCCCATTCTGCTAATGCCCAGCCTAAGTTGGG | |
| | GAGACCACTCCAGATTCCAAGATGTACAGTTTGCTTTGCTGGGCCTTTTTC | |
| | CCATGCCTGCCTTTACTCTGCCAGAGTTATATTGCTGGGGTTTTGAAGAAG | |
| | ATCCTATTAAATAAAAGAATAAGCAGTATTATTAAGTAGCCCTGCATTTCAG | |
| | GTTTCCTTGAGTGGCAGGCCAGGCCTGGCCGTGAACGTTCACTGAAATCA | |
| | TGGCCTCTTGGCCAAGATTGATAGCTTGTGCCTGTCCCTGAGTCCCAGTC | |
| | CATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAA | |
| | CCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCG | |
| | ATTTTGATTCTCAAACAAATGTGTCACAAAGTAAGGATTCTGATGTGTATAT | |
| | CACAGACAAAACTGTGCTAGACATGAGGTCTATGGACTTCAGGCTCCGGT | |
| | GCCCGTCAGTGGGCAGAGCGCACATCGCCCACAGTCCCCGAGAAGTTGG | |
| | GGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCG | |
| | CAACGGGTTTGCCGCCAGAACACAGGTAAGTGCCGTGTGTGGTTCCCGC | |
| | GGGCCTGGCCTCTTTACGGGTTATGGCCCTTGCGTGCCTTGAATTACTTC | |
| | CACTGGCTGCAGTACGTGATTCTTGATCCCGAGCTTCGGGTTGGAAGTGG | |
| | GTGGGAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAATCTGG | |
| | TGGCACCTTCGCGCCTGTCTCGCTGCTTTCGATAAGTCTCTAGCCATTTAA | |
| | AATTTTTGATGACCTGCTGCGACGCTTTTTTTCTGGCAAGATAGTCTTGTAA | |
| | ATGCGGGCCAAGATCTGCACACTGGTATTTCGGTTTTTGGGGCCGCGGG | |
| | CGGCGACGGGGCCCGTGCGTCCCAGCGCACATGTTCGGCGAGGCGGGG | |
| | CCTGCGAGCGCGGCCACCGAGAATCGGACGGGGGTAGTCTCAAGCTGG | |
| | CCGGCCTGCTCTGGTGCCTGGCCTCGCGCCGCCGTGTATCGCCCCGCCC | |
| | TGGGCGGCAAGGCTGGCCCGGTCGGCACCAGTTGCGTGAGCGGAAAGA | |
| | TGGCCGCTTCCCGGCCCTGCTGCAGGGAGCTCAAAATGGAGGACGCGGC | |
| | GCTCGGGAGAGCGGGCGGGTGAGTCACCCACACAAAGGAAAAGGGCCTT | |
| | TCCGTCCTCAGCCGTCGCTTCATGTGACTCCACGGAGTACCGGGCGCCG | |
| | TCCAGGCACCTCGATTAGTTCTCGAGCTTTTGGAGTACGTCGTCTTTAGGT | |
| | TGGGGGGAGGGGTTTTATGCGATGGAGTTTCCCCACACTGAGTGGGTGG | |
| | AGACTGAAGTTAGGCCAGCTTGGCACTTGATGTAATTCTCCTTGGAATTTG | |
| | CCCTTTTTGAGTTTGGATCTTGGTTCATTCTCAAGCCTCAGACAGTGGTTC | |
| | AAAGTTTTTTTCTTCCATTTCAGGTGTCGTGACCACCATGGCGCTTCCGGT | |
| | GACAGCACTGCTCCTCCCCTTGGCGCTGTTGCTCCACGCAGCAAGGCCG | |
| | GAGATCGTGCTGACCCAGAGCCCTGCCACACTGAGCCTGAGCCCCGGAG | |
| | AGAGGGCTACCCTGAGCTGCAGGGCCTCCCAGTCCGTGAGCATCTACCT | |
| | GGCCTGGTACCAGCAGAAACCTGGCCAGGCCCCCAGGCTGCTGATCTAC | |
| | GACGCCAGCAATAGGGCCACCGGAATCCCTGCCAGGTTTAGCGGCTCCG | |
| | GAAGCGGCACCGACTTCACCCTGACCATCTCCTCCCTGGAGCCCGAGGA | |
| | TTTCGCCGTGTACTACTGCCAGCAGAGGTCCAACTGGCCTCCCTTTACCT | |
| | TCGGCCCCGGCACCAAGGTGGATATTAAGGGCGGCGGCGGATCCGGAG | |
| | GAGGAGGCAGCGGAGGAGGAGGAAGCCAGGTGCAACTGGTGGAGTCCG | |
| | GCGGAGGCGTGGTGCAACCTGGCAGAAGCCTGAGGCTGAGCTGTGCCG | |
| | CCAGCGGCTTCACCTTCAGCAGCTACGGTATGCACTGGGTGAGGCAGGC | |
| | TCCCGGAAAGGGCCTGGAATGGGTGGCCGTGATCTGGGACGACGGCTCC | |
| | AACAAGTACTACGTGGACTCCGTGAAGGGCAGGTTCACCATCAGCAGGG | |
| | ACAACTCCAAGAACACACTGTACCTGCAGATGAACAGCCTGAGGGCCGAG | |
| | GATACCGCTGTGTATTACTGCGCCAGGGACGATTACTACGGCAGCGGCA | |
| | GCTTCAATTCCTACTACGGAACCGACGTCTGGGGCCAGGGAACCACCGT | |
| | GACCGTGAGCAGCAGTGCTGCTGCCTTTGTCCCGGTATTTCTCCCAGCCA | |
| | AACCGACCACGACTCCCGCCCCGCGCCCTCCGACACCCGCTCCCACCAT | |
| | CGCCTCTCAACCTCTTAGTCTTCGCCCCGAGGCATGCCGACCCGCCGCC | |
| | GGGGGTGCTGTTCATACGAGGGGCTTGGACTTCGCTTGTGATATTTACAT | |
| | TTGGGCTCCGTTGGCGGGTACGTGCGGCGTCCTTTTGTTGTCACTCGTTA | |
| | TTACTTTGTATTGTAATCACAGGAATCGCTCAAAGCGGAGTAGGTTGTTGC | |
| | ATTCCGATTACATGAATATGACTCCTCGCCGGCCTGGGCCGACAAGAAAA | |
| | CATTACCAACCCTATGCCCCCCCACGAGACTTCGCTGCGTACAGGTCCCG | |
| | AGTGAAGTTTTCCCGAAGCGCAGACGCTCCGGCATATCAGCAAGGACAGA | |
| | ATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAA | |
| | GAAAGAATCCCCAAGAAGGACTCTACAATGAACTCCAGAAGGATAAGATG | |
| | GCGGAGGCCTACTCAGAAATAGGTATGAAGGGCGAACGACGACGGGGAA | |
| | AAGGTCACGATGGCCTCTACCAAGGGTTGAGTACGGCAACCAAAGATACG | |
| | TACGATGCACTGCATATGCAGGCCCTGCCTCCCAGATAATAATAAAATCGC | |
| | TATCCATCGAAGATGGATGTGTGTTGGTTTTTTGTGTGTGGAGCAACAAAT | |
| | CTGACTTTGCATGTGCAAACGCCTTCAACAACAGCATTATTCCAGAAGACA | |
| | CCTTCTTCCCCAGCCCAGGTAAGGGCAGCTTTGGTGCCTTCGCAGGCTGT | |
| | TTCCTTGCTTCAGGAATGGCCAGGTTCTGCCCAGAGCTCTGGTCAATGAT | |
| | GTCTAAAACTCCTCTGATTGGTGGTCTCGGCCTTATCCATTGCCACCAAAA | |
| | CCCTCTTTTTACTAAGAAACAGTGAGCCTTGTTCTGGCAGTCCAGAGAATG | |
| | ACACGGGAAAAAAGCAGATGAAGAGAAGGTGGCAGGAGAGGGCACGTGG | |
| | CCCAGCCTCAGTCTCTCCAACTGAGTTCCTGCCTGCCTGCCTTTGCTCAG | |
| | ACTGTTTGCCCCTTACTGCTCTTCTAGGCCTCATTCTAAGCCCCTTCTCCA | |
| | AGTTGCCTCTCCTTATTTCTCCCTGTCTGCCAAAAAATCTTTCCCAGCTCA | |
| | CTAAGTCAGTCTCACGCAGTCACTCATTAACCCACCAATCACTGATTGTGC | |
| | CGGCACATGAATGCACCAGGTGTTGAAGTGGAGGAATTAAAAAGTCAGAT | |
| | GAGGGGTGTGCCCAGAGGAAGCACCATTCTAGTTGGGGGAGCCCATCTG | |
| | TCAGCTGGGAAAAGTCCAAATAACTTCAGATTGGAATGTGTTTTAACTCAG | |
| | GGTTGAGAAAACAGCTACCTTCAGGACAAAAGTCAGGGAAGGGCTCTCTG | |
| | AAGAAATGCTACTTGAAGATACCAGCCCTACCAAGGGCAGGGAGAGGAC | |
| | CCTATAGAGGCCTGGGACAGGAGCTCAATGAGAAAGG | |

PTK7-13

| | | |
|---|---|---|
| PKT7-13 CAR | ATGGCGCTGCCGGTGACCGCGCTGCTGCTGCCGCTGGCGCTGCTGCTGC | 72 |
| | ATGCGGCGCGCCCGGAAATTGTGCTGACCCAGAGCCCGGGCACCCTGAG | |
| | CCTGAGCCCGGGCGAACGCGCGACCCTGAGCTGCCGCGCGAGCCAGAG | |
| | CGTGAGCAGCAGCTATCTGGCGTGGTATCAGCAGAAACCGGGCCAGGCG | |
| | CCGCGCCTGCTGATTTATGGCGCGAGCAGCCGCGCGACCGGCATTCCGG | |
| | ATCGCTTTAGCGGCAGCGGCAGCGGCACCGATTTTACCCTGACCATTAGC | |
| | CGCCTGGAACCGGAAGATTTTGCGGTGTATTATTGCCAGCAGTATGGCAG | |
| | CAGCCCGATGTATACCTTTGGCCAGGGCACCAAACTGGAAATTAAAGGCG | |
| | GCGGCGGCAGCGGCGGCGGCGGCAGCGGCGGCGGCGGCAGCGAAGTG | |
| | CAGCTGGTGCAGAGCGGCGGCGGCCTGGTGCATCCGGGCGGCAGCCTG | |
| | CGCCTGAGCTGCGCGGGCAGCGGCTTTACCTTTAGCACCTATCTGATGTA | |
| | TTGGGTGCGCCAGGCGCCGGGCAAAACCCTGGAATGGGTGAGCGCGATT | |
| | GGCAGCGGCGGCGATACCTATTATGCGGATAGCGTGAAAGGCCGCTTTA | |
| | CCATTAGCCGCGATAACGCGAAAAACAGCCTGTATCTGCAGATGAACAGC | |
| | CTGCGCGCGGAAGATATGGCGGTGTATTATTGCGCGCGCGGCCTGGGCT | |
| | ATTGGGGCCAGGGCACCCTGGTGACCGTGAGCAGCGCGGCGGCGTTTGT | |
| | GCCGGTGTTTCTGCCGGCGAAACCGACCACCACCCCGGCGCCGCGCCC | |
| | GCCGACCCCGGCGCCGACCATTGCGAGCCAGCCGCTGAGCCTGCGCCC | |
| | GGAAGCGTGCCGCCCGGCGGCGGGCGGCGCGGTGCATACCCGCGGCCT | |
| | GGATTTTGCGTGCGATATTTATATTTGGGCGCCGCTGGCGGGCACCTGCG | |
| | GCGTGCTGCTGCTGAGCCTGGTGATTACCCTGTATTGCAACCATCGCAAC | |
| | CGCAGCAAACGCAGCCGCCTGCTGCATAGCGATTATATGAACATGACCCC | |
| | GCGCCGCCCGGGCCCGACCCGCAAACATTATCAGCCGTATGCGCCGCCG | |
| | CGCGATTTTGCGGCGTATCGCAGCCGCGTGAAATTTAGCCGCAGCGCGG | |
| | ATGCGCCGGCGTATCAGCAGGGCCAGAACCAGCTGTATAACGAACTGAA | |
| | CCTGGGCCGCCGCGAAGAATATGATGTGCTGGATAAACGCCGCGGCCGC | |
| | GATCCGGAAATGGGCGGCAAACCGCGCCGCAAAAACCCGCAGGAAGGC | |
| | CTGTATAACGAACTGCAGAAAGATAAAATGGCGGAAGCGTATAGCGAAAT | |
| | TGGCATGAAAGGCGAACGCCGCCGCGGCAAAGGCCATGATGGCCTGTAT | |
| | CAGGGCCTGAGCACCGCGACCAAAGATACCTATGATGCGCTGCATATGCA | |
| | GGCGCTGCCGCCGCGC | |
| PKT7-13 CAR | MALPVTALLLPLALLLHAARPEIVLTQSPGTLSLSPGERATLSCRASQSVSSSY | 73 |
| | LAWYQQKPGQAPRLLIYGASSRATGIPDRFSGSGSGTDFTLTISRLEPEDFAV | |
| | YYCQQYGSSPMYTFGQGTKLEIKGGGGSGGGGSGGGGSEVQLVQSGGGL | |
| | VHPGGSLRLSCAGSGFTFSTYLMYWVRQAPGKTLEWVSAIGSGGDTYYADS | |
| | VKGRFTISRDNAKNSLYLQMNSLRAEDMAVYYCARGLGYWGQGTLVTVSSA | |
| | AAFVPVFLPAKPTTTPAPRPPTPAPTIASQPLSLRPEACRPAAGGAVHTRGLD | |
| | FACDIYIWAPLAGTCGVLLLSLVITLYCNHRNRSKRSRLLHSDYMNMTPRRPG | |
| | PTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERR | |
| | RGKGHDGLYQGLSTATKDTYDALHMQALPPR | |
| PTK7-13 scFv | GAAATTGTGCTGACCCAGAGCCCGGGCACCCTGAGCCTGAGCCCGGGCG | 74 |
| | AACGCGCGACCCTGAGCTGCCGCGCGAGCCAGAGCGTGAGCAGCAGCT | |
| | ATCTGGCGTGGTATCAGCAGAAACCGGGCCAGGCGCCGCGCCTGCTGAT | |
| | TTATGGCGCGAGCAGCCGCGCGACCGGCATTCCGGATCGCTTTAGCGGC | |
| | AGCGGCAGCGGCACCGATTTTACCCTGACCATTAGCCGCCTGGAACCGG | |
| | AAGATTTTGCGGTGTATTATTGCCAGCAGTATGGCAGCAGCCCGATGTATA | |
| | CCTTTGGCCAGGGCACCAAACTGGAAATTAAAGGCGGCGGCGGCAGCGG | |
| | CGGCGGCGGCAGCGGCGGCGGCGGCAGCGAAGTGCAGCTGGTGCAGA | |
| | GCGGCGGCGGCCTGGTGCATCCGGGCGGCAGCCTGCGCCTGAGCTGCG | |
| | CGGGCAGCGGCTTTACCTTTAGCACCTATCTGATGTATTGGGTGCGCCAG | |
| | GCGCCGGGCAAAACCCTGGAATGGGTGAGCGCGATTGGCAGCGGCGGC | |
| | GATACCTATTATGCGGATAGCGTGAAAGGCCGCTTTACCATTAGCCGCGA | |
| | TAACGCGAAAAACAGCCTGTATCTGCAGATGAACAGCCTGCGCGCGGAA | |
| | GATATGGCGGTGTATTATTGCGCGCGCGGCCTGGGCTATTGGGGCCAGG | |
| | GCACCCTGGTGACCGTGAGCAGC | |
| PTK7-13 scFv | EIVLTQSPGTLSLSPGERATLSCRASQSVSSSYLAWYQQKPGQAPRLLIYGAS | 75 |
| (linker | SRATGIPDRFSGSGSGTDFTLTISRLEPEDFAVYYCQQYGSSPMYTFGQGTK | |
| underlined) | LEIKGGGGSGGGGSGGGGSEVQLVQSGGGLVHPGGSLRLSCAGSGFTFST | |
| | YLMYWVRQAPGKTLEWVSAIGSGGDTYYADSVKGRFTISRDNAKNSLYLQM | |
| | NSLRAEDMAVYYCARGLGYWGQGTLVTVSS | |
| PTK7-13 scFv | EVQLVQSGGGLVHPGGSLRLSCAGSGFTFSTYLMYWVRQAPGKTLEWVSAI | 76 |
| VH | GSGGDTYYADSVKGRFTISRDNAKNSLYLQMNSLRAEDMAVYYCARGLGYW | |
| | GQGTLVTVSS | |
| PTK7-13 scFv | EIVLTQSPGTLSLSPGERATLSCRASQSVSSSYLAWYQQKPGQAPRLLIYGAS | 77 |
| VL | SRATGIPDRFSGSGSGTDFTLTISRLEPEDFAVYYCQQYGSSPMYTFGQGTK | |
| | LEIK | |
| PTK7-13 | GAGATGTAAGGAGCTGCTGTGACTTGCTCAAGGCCTTATATCGAGTAAAC | 78 |
| Donor | GGTAGTGCTGGGGCTTAGACGCAGGTGTTCTGATTTATAGTTCAAAACCT | |
| LHA to RHA | CTATCAATGAGAGAGCAATCTCCTGGTAATGTGATAGATTTCCCAACTTAA | |
| | TGCCAACATACCATAAACCTCCCATTCTGCTAATGCCCAGCCTAAGTTGGG | |
| | GAGACCACTCCAGATTCCAAGATGTACAGTTTGCTTTGCTGGGCCTTTTTC | |
| | CCATGCCTGCCTTTACTCTGCCAGAGTTATATTGCTGGGGTTTTGAAGAAG | |
| | ATCCTATTAAATAAAAGAATAAGCAGTATTATTAAGTAGCCCTGCATTTCAG | |
| | GTTTCCTTGAGTGGCAGGCCAGGCCTGGCCGTGAACGTTCACTGAAATCA | |
| | TGGCCTCTTGGCCAAGATTGATAGCTTGTGCCTGTCCCTGAGTCCCAGTC | |
| | CATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAA | |
| | CCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCG | |
| | ATTTTGATTCTCAAACAAATGTGTCACAAAGTAAGGATTCTGATGTGTATAT | |
| | CACAGACAAAACTGTGCTAGACATGAGGTCTATGGACTTCAGGCTCCGGT | |
| | GCCCGTCAGTGGGCAGAGCGCACATCGCCCACAGTCCCCGAGAAGTTGG | |
| | GGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCG | |
| | CAACGGGTTTGCCGCCAGAACACAGGTAAGTGCCGTGTGTGGTTCCCGC | |
| | GGGCCTGGCCTCTTTACGGGTTATGGCCCTTGCGTGCCTTGAATTACTTC | |
| | CACTGGCTGCAGTACGTGATTCTTGATCCCGAGCTTCGGGTTGGAAGTGG | |
| | GTGGGAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAATCTGG | |
| | TGGCACCTTCGCGCCTGTCTCGCTGCTTTCGATAAGTCTCTAGCCATTTAA | |
| | AATTTTTGATGACCTGCTGCGACGCTTTTTTTCTGGCAAGATAGTCTTGTAA | |
| | ATGCGGGCCAAGATCTGCACACTGGTATTTCGGTTTTTGGGGCCGCGGG | |
| | CGGCGACGGGGCCCGTGCGTCCCAGCGCACATGTTCGGCGAGGCGGGG | |
| | CCTGCGAGCGCGGCCACCGAGAATCGGACGGGGGTAGTCTCAAGCTGG | |
| | CCGGCCTGCTCTGGTGCCTGGCCTCGCGCCGCCGTGTATCGCCCCGCCC | |
| | TGGGCGGCAAGGCTGGCCCGGTCGGCACCAGTTGCGTGAGCGGAAAGA | |
| | TGGCCGCTTCCCGGCCCTGCTGCAGGGAGCTCAAAATGGAGGACGCGGC | |
| | GCTCGGGAGAGCGGGCGGGTGAGTCACCCACACAAAGGAAAAGGGCCTT | |
| | TCCGTCCTCAGCCGTCGCTTCATGTGACTCCACGGAGTACCGGGCGCCG | |
| | TCCAGGCACCTCGATTAGTTCTCGAGCTTTTGGAGTACGTCGTCTTTAGGT | |
| | TGGGGGGAGGGGTTTTATGCGATGGAGTTTCCCCACACTGAGTGGGTGG | |
| | AGACTGAAGTTAGGCCAGCTTGGCACTTGATGTAATTCTCCTTGGAATTTG | |
| | CCCTTTTTGAGTTTGGATCTTGGTTCATTCTCAAGCCTCAGACAGTGGTTC | |
| | AAAGTTTTTTTCTTCCATTTCAGGTGTCGTGACCACCATGGCGCTTCCGGT | |
| | GACAGCACTGCTCCTCCCCTTGGCGCTGTTGCTCCACGCAGCAAGGCCG | |
| | GAGATCGTGCTGACCCAGAGCCCCGGAACACTGAGCCTGTCCCCCGGAG | |
| | AAAGAGCCACACTGTCCTGCAGGGCCAGCCAGAGCGTGAGCAGCTCCTA | |
| | CCTGGCCTGGTACCAGCAGAAGCCTGGACAGGCCCCCAGGCTGCTGATT | |
| | TACGGCGCCAGCAGCAGGGCCACCGGCATCCCCGACAGATTCAGCGGAT | |
| | CCGGCAGCGGCACCGACTTCACCCTGACCATCAGCAGGCTGGAGCCCGA | |
| | GGACTTCGCTGTGTACTACTGCCAGCAGTACGGCAGCAGCCCCATGTACA | |
| | CCTTCGGCCAGGGCACCAAGCTGGAGATCAAGGGAGGAGGAGGATCCG | |
| | GAGGAGGCGGAAGCGGAGGAGGAGGAAGCGAGGTGCAGCTGGTGCAGA | |
| | GCGGCGGAGGACTGGTGCATCCCGGAGGATCCCTGAGACTGAGCTGTGC | |
| | CGGCAGCGGATTCACATTCTCCACCTACCTGATGTACTGGGTGAGGCAGG | |
| | CCCCTGGCAAGACCCTGGAGTGGGTGTCCGCCATTGGCTCCGGCGGAGA | |
| | CACCTATTATGCCGACTCCGTCAAGGGCAGGTTCACCATCAGCAGAGACA | |
| | ACGCCAAGAACTCCCTGTACCTGCAGATGAACAGCCTGAGGGCCGAGGA | |
| | TATGGCTGTGTACTATTGCGCTAGGGGCCTGGGATACTGGGGCCAGGGA | |
| | ACCCTGGTGACCGTGAGCTCCAGTGCTGCTGCCTTTGTCCCGGTATTTCT | |
| | CCCAGCCAAACCGACCACGACTCCCGCCCCGCGCCCTCCGACACCCGCT | |
| | CCCACCATCGCCTCTCAACCTCTTAGTCTTCGCCCCGAGGCATGCCGACC | |
| | CGCCGCCGGGGGTGCTGTTCATACGAGGGGCTTGGACTTCGCTTGTGAT | |
| | ATTTACATTTGGGCTCCGTTGGCGGGTACGTGCGGCGTCCTTTTGTTGTC | |
| | ACTCGTTATTACTTTGTATTGTAATCACAGGAATCGCTCAAAGCGGAGTAG | |
| | GTTGTTGCATTCCGATTACATGAATATGACTCCTCGCCGGCCTGGGCCGA | |
| | CAAGAAAACATTACCAACCCTATGCCCCCCCACGAGACTTCGCTGCGTAC | |
| | AGGTCCCGAGTGAAGTTTTCCCGAAGCGCAGACGCTCCGGCATATCAGCA | |
| | AGGACAGAATCAGCTGTATAACGAACTGAATTTGGGACGCCGCGAGGAGT | |
| | ATGACGTGCTTGATAAACGCCGGGGGAGAGACCCGGAAATGGGGGGTAA | |
| | ACCCCGAAGAAAGAATCCCCAAGAAGGACTCTACAATGAACTCCAGAAGG | |
| | ATAAGATGGCGGAGGCCTACTCAGAAATAGGTATGAAGGGCGAACGACG | |
| | ACGGGGAAAAGGTCACGATGGCCTCTACCAAGGGTTGAGTACGGCAACC | |
| | AAAGATACGTACGATGCACTGCATATGCAGGCCCTGCCTCCCAGATAATA | |
| | ATAAAATCGCTATCCATCGAAGATGGATGTGTGTTGGTTTTTTGTGTGTGG | |
| | AGCAACAAATCTGACTTTGCATGTGCAAACGCCTTCAACAACAGCATTATT | |
| | CCAGAAGACACCTTCTTCCCCAGCCCAGGTAAGGGCAGCTTTGGTGCCTT | |
| | CGCAGGCTGTTTCCTTGCTTCAGGAATGGCCAGGTTCTGCCCAGAGCTCT | |
| | GGTCAATGATGTCTAAAACTCCTCTGATTGGTGGTCTCGGCCTTATCCATT | |
| | GCCACCAAAACCCTCTTTTTACTAAGAAACAGTGAGCCTTGTTCTGGCAGT | |
| | CCAGAGAATGACACGGGAAAAAAGCAGATGAAGAGAAGGTGGCAGGAGA | |
| | GGGCACGTGGCCCAGCCTCAGTCTCTCCAACTGAGTTCCTGCCTGCCTG | |
| | CCTTTGCTCAGACTGTTTGCCCCTTACTGCTCTTCTAGGCCTCATTCTAAG | |
| | CCCCTTCTCCAAGTTGCCTCTCCTTATTTCTCCCTGTCTGCCAAAAAATCTT | |
| | TCCCAGCTCACTAAGTCAGTCTCACGCAGTCACTCATTAACCCACCAATCA | |
| | CTGATTGTGCCGGCACATGAATGCACCAGGTGTTGAAGTGGAGGAATTAA | |
| | AAAGTCAGATGAGGGGTGTGCCCAGAGGAAGCACCATTCTAGTTGGGGG | |
| | AGCCCATCTGTCAGCTGGGAAAAGTCCAAATAACTTCAGATTGGAATGTGT | |
| | TTTAACTCAGGGTTGAGAAAACAGCTACCTTCAGGACAAAAGTCAGGGAA | |
| | GGGCTCTCTGAAGAAATGCTACTTGAAGATACCAGCCCTACCAAGGGCAG | |
| | GGAGAGGACCCTATAGAGGCCTGGGACAGGAGCTCAATGAGAAAGG | |

PTK7-17

| | | |
|---|---|---|
| PKT7-17 CAR | ATGGCGCTGCCGGTGACCGCGCTGCTGCTGCCGCTGGCGCTGCTGCTGC | 79 |
| | ATGCGGCGCGCCCGGAAGTGCAGCTGGTGCAGAGCGGCGGCGGCCTGG | |
| | TGCATCCGGGCGGCAGCCTGCGCCTGAGCTGCGCGGGCAGCGGCTTTA | |
| | CCTTTAGCACCTATCTGATGTATTGGGTGCGCCAGGCGCCGGGCAAAACC | |
| | CTGGAATGGGTGAGCGCGATTGGCAGCGGCGGCGATACCTATTATGCGG | |
| | ATAGCGTGAAAGGCCGCTTTACCATTAGCCGCGATAACGCGAAAAACAGC | |
| | CTGTATCTGCAGATGAACAGCCTGCGCGCGGAAGATATGGCGGTGTATTA | |
| | TTGCGCGCGCGGCCTGGGCTATTGGGGCCAGGGCACCCTGGTGACCGT | |
| | GAGCAGCGGCGGCGGCGGCAGCGGCGGCGGCGGCAGCGGCGGCGGC | |
| | GGCAGCGAAATTGTGCTGACCCAGAGCCCGGGCACCCTGAGCCTGAGCC | |
| | CGGGCGAACGCGCGACCCTGAGCTGCCGCGCGAGCCAGAGCGTGAGCA | |
| | GCAGCTATCTGGCGTGGTATCAGCAGAAACCGGGCCAGGCGCCGCGCCT | |
| | GCTGATTTATGGCGCGAGCAGCCGCGCGACCGGCATTCCGGATCGCTTT | |
| | AGCGGCAGCGGCAGCGGCACCGATTTTACCCTGACCATTAGCCGCCTGG | |
| | AACCGGAAGATTTTGCGGTGTATTATTGCCAGCAGTATGGCAGCAGCCCG | |
| | ATGTATACCTTTGGCCAGGGCACCAAACTGGAAATTAAAAGCGCGGCGGC | |
| | GTTTGTGCCGGTGTTTCTGCCGGCGAAACCGACCACCACCCCGGCGCCG | |
| | CGCCCGCCGACCCCGGCGCCGACCATTGCGAGCCAGCCGCTGAGCCTG | |
| | CGCCCGGAAGCGTGCCGCCCGGCGGCGGGCGGCGCGGTGCATACCCG | |
| | CGGCCTGGATTTTGCGTGCGATATTTATATTTGGGCGCCGCTGGCGGGCA | |
| | CCTGCGGCGTGCTGCTGCTGAGCCTGGTGATTACCCTGTATTGCAACCAT | |
| | CGCAACCGCAGCAAACGCAGCCGCCTGCTGCATAGCGATTATATGAACAT | |
| | GACCCCGCGCCGCCCGGGCCCGACCCGCAAACATTATCAGCCGTATGCG | |
| | CCGCCGCGCGATTTTGCGGCGTATCGCAGCCGCGTGAAATTTAGCCGCA | |
| | GCGCGGATGCGCCGGCGTATCAGCAGGGCCAGAACCAGCTGTATAACGA | |
| | ACTGAACCTGGGCCGCCGCGAAGAATATGATGTGCTGGATAAACGCCGC | |
| | GGCCGCGATCCGGAAATGGGCGGCAAACCGCGCCGCAAAAACCCGCAG | |
| | GAAGGCCTGTATAACGAACTGCAGAAAGATAAAATGGCGGAAGCGTATAG | |
| | CGAAATTGGCATGAAAGGCGAACGCCGCCGCGGCAAAGGCCATGATGGC | |
| | CTGTATCAGGGCCTGAGCACCGCGACCAAAGATACCTATGATGCGCTGCA | |
| | TATGCAGGCGCTGCCGCCGCGC | |
| PKT7-17 CAR | MALPVTALLLPLALLLHAARPEVQLVQSGGGLVHPGGSLRLSCAGSGFTFSTY | 80 |
| | LMYWVRQAPGKTLEWVSAIGSGGDTYYADSVKGRFTISRDNAKNSLYLQMN | |
| | SLRAEDMAVYYCARGLGYWGQGTLVTVSSGGGGSGGGGSGGGGSEIVLTQ | |
| | SPGTLSLSPGERATLSCRASQSVSSSYLAWYQQKPGQAPRLLIYGASSRATG | |
| | IPDRFSGSGSGTDFTLTISRLEPEDFAVYYCQQYGSSPMYTFGQGTKLEIKSA | |
| | AAFVPVFLPAKPTTTPAPRPPTPAPTIASQPLSLRPEACRPAAGGAVHTRGLD | |
| | FACDIYIWAPLAGTCGVLLLSLVITLYCNHRNRSKRSRLLHSDYMNMTPRRPG | |
| | PTRKHYQPYAPPRDFAAYRSRVKFSRSADAPAYQQGQNQLYNELNLGRREE | |
| | YDVLDKRRGRDPEMGGKPRRKNPQEGLYNELQKDKMAEAYSEIGMKGERR | |
| | RGKGHDGLYQGLSTATKDTYDALHMQALPPR | |
| PTK7-17 scFv | GAAGTGCAGCTGGTGCAGAGCGGCGGCGGCCTGGTGCATCCGGGCGGC | 81 |
| | AGCCTGCGCCTGAGCTGCGCGGGCAGCGGCTTTACCTTTAGCACCTATCT | |
| | GATGTATTGGGTGCGCCAGGCGCCGGGCAAAACCCTGGAATGGGTGAGC | |
| | GCGATTGGCAGCGGCGGCGATACCTATTATGCGGATAGCGTGAAAGGCC | |
| | GCTTTACCATTAGCCGCGATAACGCGAAAAACAGCCTGTATCTGCAGATG | |
| | AACAGCCTGCGCGCGGAAGATATGGCGGTGTATTATTGCGCGCGCGGCC | |
| | TGGGCTATTGGGGCCAGGGCACCCTGGTGACCGTGAGCAGCGGCGGCG | |
| | GCGGCAGCGGCGGCGGCGGCAGCGGCGGCGGCGGCAGCGAAATTGTG | |
| | CTGACCCAGAGCCCGGGCACCCTGAGCCTGAGCCCGGGCGAACGCGCG | |
| | ACCCTGAGCTGCCGCGCGAGCCAGAGCGTGAGCAGCAGCTATCTGGCGT | |
| | GGTATCAGCAGAAACCGGGCCAGGCGCCGCGCCTGCTGATTTATGGCGC | |
| | GAGCAGCCGCGCGACCGGCATTCCGGATCGCTTTAGCGGCAGCGGCAG | |
| | CGGCACCGATTTTACCCTGACCATTAGCCGCCTGGAACCGGAAGATTTTG | |
| | CGGTGTATTATTGCCAGCAGTATGGCAGCAGCCCGATGTATACCTTTGGC | |
| | CAGGGCACCAAACTGGAAATTAAA | |
| PTK7-17 scFv | EVQLVQSGGGLVHPGGSLRLSCAGSGFTFSTYLMYWVRQAPGKTLEWVSAI | 82 |
| (linker | GSGGDTYYADSVKGRFTISRDNAKNSLYLQMNSLRAEDMAVYYCARGLGYW | |
| underlined) | GQGTLVTVSSGGGGSGGGGSGGGGSEIVLTQSPGTLSLSPGERATLSCRAS | |
| | QSVSSSYLAWYQQKPGQAPRLLIYGASSRATGIPDRFSGSGSGTDFTLTISRL | |
| | EPEDFAVYYCQQYGSSPMYTFGQGTKLEIK | |
| PTK7-17 scFv | EVQLVQSGGGLVHPGGSLRLSCAGSGFTFSTYLMYWVRQAPGKTLEWVSAI | 83 |
| VH | GSGGDTYYADSVKGRFTISRDNAKNSLYLQMNSLRAEDMAVYYCARGLGY | |
| | WGQGTLVTVSS | |
| PTK7-17 scFv | EIVLTQSPGTLSLSPGERATLSCRASQSVSSSYLAWYQQKPGQAPRLLIYGAS | 84 |
| VL | SRATGIPDRFSGSGSGTDFTLTISRLEPEDFAVYYCQQYGSSPMYTFGQGTK | |
| | LEIK | |
| PTK7-17 | TYLMY | 85 |
| VH CDR1 | | |
| PTK7-17 | AIGSGGDTYYADSVKG | 86 |
| VH CDR2 | | |
| PTK7-17 | GLGY | 87 |
| VH CDR3 | | |
| PTK7-17 | RASQSVSSSYLA | 88 |
| VL CDR1 | | |
| PTK7-17 | GASSRAT | 89 |
| VL CDR2 | | |
| PTK7-17 | QQYGSSPMYT | 90 |
| VL CDR3 | | |
| PTK-17 | GAGATGTAAGGAGCTGCTGTGACTTGCTCAAGGCCTTATATCGAGTAAAC | 91 |
| Donor | GGTAGTGCTGGGGCTTAGACGCAGGTGTTCTGATTTATAGTTCAAAACCT | |
| LHA to RHA | CTATCAATGAGAGAGCAATCTCCTGGTAATGTGATAGATTTCCCAACTTAA | |
| | TGCCAACATACCATAAACCTCCCATTCTGCTAATGCCCAGCCTAAGTTGGG | |
| | GAGACCACTCCAGATTCCAAGATGTACAGTTTGCTTTGCTGGGCCTTTTTC | |
| | CCATGCCTGCCTTTACTCTGCCAGAGTTATATTGCTGGGGTTTTGAAGAAG | |
| | ATCCTATTAAATAAAAGAATAAGCAGTATTATTAAGTAGCCCTGCATTTCAG | |
| | GTTTCCTTGAGTGGCAGGCCAGGCCTGGCCGTGAACGTTCACTGAAATCA | |
| | TGGCCTCTTGGCCAAGATTGATAGCTTGTGCCTGTCCCTGAGTCCCAGTC | |
| | CATCACGAGCAGCTGGTTTCTAAGATGCTATTTCCCGTATAAAGCATGAGA | |
| | CCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAA | |
| | CCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCG | |
| | ATTTTGATTCTCAAACAAATGTGTCACAAAGTAAGGATTCTGATGTGTATAT | |
| | CACAGACAAAACTGTGCTAGACATGAGGTCTATGGACTTCAGGCTCCGGT | |
| | GCCCGTCAGTGGGCAGAGCGCACATCGCCCACAGTCCCCGAGAAGTTGG | |
| | GGGGAGGGGTCGGCAATTGAACCGGTGCCTAGAGAAGGTGGCGCGGGG | |
| | TAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCG | |
| | CAACGGGTTTGCCGCCAGAACACAGGTAAGTGCCGTGTGTGGTTCCCGC | |
| | GGGCCTGGCCTCTTTACGGGTTATGGCCCTTGCGTGCCTTGAATTACTTC | |
| | CACTGGCTGCAGTACGTGATTCTTGATCCCGAGCTTCGGGTTGGAAGTGG | |
| | GTGGGAGAGTTCGAGGCCTTGCGCTTAAGGAGCCCCTTCGCCTCGTGCT | |
| | TGAGTTGAGGCCTGGCCTGGGCGCTGGGGCCGCCGCGTGCGAATCTGG | |
| | TGGCACCTTCGCGCCTGTCTCGCTGCTTTCGATAAGTCTCTAGCCATTTAA | |
| | AATTTTTGATGACCTGCTGCGACGCTTTTTTTCTGGCAAGATAGTCTTGTAA | |
| | ATGCGGGCCAAGATCTGCACACTGGTATTTCGGTTTTTGGGGCCGCGGG | |
| | CGGCGACGGGGCCCGTGCGTCCCAGCGCACATGTTCGGCGAGGCGGGG | |
| | CCTGCGAGCGCGGCCACCGAGAATCGGACGGGGGTAGTCTCAAGCTGG | |
| | CCGGCCTGCTCTGGTGCCTGGCCTCGCGCCGCCGTGTATCGCCCCGCCC | |
| | TGGGCGGCAAGGCTGGCCCGGTCGGCACCAGTTGCGTGAGCGGAAAGA | |
| | TGGCCGCTTCCCGGCCCTGCTGCAGGGAGCTCAAAATGGAGGACGCGGC | |
| | GCTCGGGAGAGCGGGCGGGTGAGTCACCCACACAAAGGAAAAGGGCCTT | |
| | TCCGTCCTCAGCCGTCGCTTCATGTGACTCCACGGAGTACCGGGCGCCG | |
| | TCCAGGCACCTCGATTAGTTCTCGAGCTTTTGGAGTACGTCGTCTTTAGGT | |
| | TGGGGGGAGGGGTTTTATGCGATGGAGTTTCCCCACACTGAGTGGGTGG | |
| | AGACTGAAGTTAGGCCAGCTTGGCACTTGATGTAATTCTCCTTGGAATTTG | |
| | CCCTTTTTGAGTTTGGATCTTGGTTCATTCTCAAGCCTCAGACAGTGGTTC | |
| | AAAGTTTTTTTCTTCCATTTCAGGTGTCGTGACCACCATGGCGCTTCCGGT | |
| | GACAGCACTGCTCCTCCCCTTGGCGCTGTTGCTCCACGCAGCAAGGCCG | |
| | GAGGTCCAGCTGGTGCAGAGCGGAGGCGGACTGGTGCATCCTGGAGGC | |
| | TCCCTGAGACTGTCCTGTGCCGGCAGCGGCTTCACCTTCAGCACCTACCT | |
| | GATGTACTGGGTGAGACAGGCCCCCGGCAAAACCCTGGAGTGGGTGAGC | |
| | GCTATCGGCAGCGGCGGAGACACATACTACGCCGACAGCGTGAAGGGCA | |
| | GGTTCACCATCAGCAGGGACAACGCCAAGAACTCCCTGTACCTGCAGATG | |
| | AACTCCCTGAGGGCTGAGGACATGGCCGTGTACTACTGCGCTAGAGGCC | |
| | TGGGCTACTGGGGACAGGGCACACTGGTGACAGTGAGCAGCGGAGGCG | |
| | GCGGCAGCGGAGGCGGCGGCAGCGGCGGCGGAGGCAGCGAGATCGTG | |
| | CTGACACAGAGCCCTGGCACCCTGTCCCTGTCCCCTGGCGAAAGGGCCA | |
| | CCCTGAGCTGTAGGGCCAGCCAGTCCGTGAGCAGCAGCTATCTGGCCTG | |
| | GTACCAGCAGAAACCCGGCCAGGCCCCTAGACTGCTGATCTACGGCGCC | |
| | TCCTCCAGAGCCACCGGAATCCCCGATAGATTCAGCGGCTCCGGCAGCG | |
| | GCACCGATTTCACACTGACCATCAGCAGGCTGGAGCCCGAGGACTTCGC | |
| | CGTGTATTACTGCCAGCAGTACGGCAGCAGCCCTATGTACACATTCGGCC | |
| | AGGGCACCAAGCTGGAGATCAAGAGTGCTGCTGCCTTTGTCCCGGTATTT | |
| | CTCCCAGCCAAACCGACCACGACTCCCGCCCCGCGCCCTCCGACACCCG | |
| | CTCCCACCATCGCCTCTCAACCTCTTAGTCTTCGCCCCGAGGCATGCCGA | |
| | CCCGCCGCCGGGGGTGCTGTTCATACGAGGGGCTTGGACTTCGCTTGTG | |
| | ATATTTACATTTGGGCTCCGTTGGCGGGTACGTGCGGCGTCCTTTTGTTGT | |
| | CACTCGTTATTACTTTGTATTGTAATCACAGGAATCGCTCAAAGCGGAGTA | |
| | GGTTGTTGCATTCCGATTACATGAATATGACTCCTCGCCGGCCTGGGCCG | |
| | ACAAGAAAACATTACCAACCCTATGCCCCCCCACGAGACTTCGCTGCGTA | |
| | CAGGTCCCGAGTGAAGTTTTCCCGAAGCGCAGACGCTCCGGCATATCAG | |
| | CAAGGACAGAATCAGCTGTATAACGAACTGAATTTGGGACGCCGCGAGGA | |
| | GTATGACGTGCTTGATAAACGCCGGGGGAGAGACCCGGAAATGGGGGGT | |
| | AAACCCCGAAGAAAGAATCCCCAAGAAGGACTCTACAATGAACTCCAGAA | |
| | GGATAAGATGGCGGAGGCCTACTCAGAAATAGGTATGAAGGGCGAACGA | |
| | CGACGGGGAAAAGGTCACGATGGCCTCTACCAAGGGTTGAGTACGGCAA | |
| | CCAAAGATACGTACGATGCACTGCATATGCAGGCCCTGCCTCCCAGATAA | |
| | TAATAAAATCGCTATCCATCGAAGATGGATGTGTGTTGGTTTTTTGTGTGT | |
| | GGAGCAACAAATCTGACTTTGCATGTGCAAACGCCTTCAACAACAGCATTA | |
| | TTCCAGAAGACACCTTCTTCCCCAGCCCAGGTAAGGGCAGCTTTGGTGCC | |
| | TTCGCAGGCTGTTTCCTTGCTTCAGGAATGGCCAGGTTCTGCCCAGAGCT | |
| | CTGGTCAATGATGTCTAAAACTCCTCTGATTGGTGGTCTCGGCCTTATCCA | |
| | TTGCCACCAAAACCCTCTTTTTACTAAGAAACAGTGAGCCTTGTTCTGGCA | |
| | GTCCAGAGAATGACACGGGAAAAAAGCAGATGAAGAGAAGGTGGCAGGA | |
| | GAGGGCACGTGGCCCAGCCTCAGTCTCTCCAACTGAGTTCCTGCCTGCCT | |
| | GCCTTTGCTCAGACTGTTTGCCCCTTACTGCTCTTCTAGGCCTCATTCTAA | |
| | GCCCCTTCTCCAAGTTGCCTCTCCTTATTTCTCCCTGTCTGCCAAAAAATC | |
| | TTTCCCAGCTCACTAAGTCAGTCTCACGCAGTCACTCATTAACCCACCAAT | |
| | CACTGATTGTGCCGGCACATGAATGCACCAGGTGTTGAAGTGGAGGAATT | |
| | AAAAAGTCAGATGAGGGGTGTGCCCAGAGGAAGCACCATTCTAGTTGGG | |
| | GGAGCCCATCTGTCAGCTGGGAAAAGTCCAAATAACTTCAGATTGGAATG | |
| | TGTTTTAACTCAGGGTTGAGAAAACAGCTACCTTCAGGACAAAAGTCAGG | |
| | GAAGGGCTCTCTGAAGAAATGCTACTTGAAGATACCAGCCCTACCAAGGG | |
| | CAGGGAGAGGACCCTATAGAGGCCTGGGACAGGAGCTCAATGAGAAAGG | |
| CD8 signal | MALPVTALLLPLALLLHAARP | 93 |
| peptide | | |
| CD8a | GCTGCTGCCTTTGTCCCGGTATTTCTCCCAGCCAAACCGACCACGACTCC | 94 |
| transmembrane + | CGCCCCGCGCCCTCCGACACCCGCTCCCACCATCGCCTCTCAACCTCTTA | |
| 5′ Linker | GTCTTCGCCCCGAGGCATGCCGACCCGCCGCCGGGGGTGCTGTTCATAC | |
| (underlined) | GAGGGGCTTGGACTTCGCTTGTGATATTTACATTTGGGCTCCGTTGGCGG | |
| | GTACGTGCGGCGTCCTTTTGTTGTCACTCGTTATTACTTTGTATTGTAATCA | |
| | CAGGAATCGC | |
| CD8a | SAAAFVPVFLPAKPTTTPAPRPPTPAPTIASQPLSLRPEACRPAAGGAVHTRG | 95 |
| transmembrane + | LDFACDIYIWAPLAGTCGVLLLSLVITLYCNHRNR | |
| 5′ Linker | | |
| (underlined) | | |
| CD8a | TTTGTCCCGGTATTTCTCCCAGCCAAACCGACCACGACTCCCGCCCCGCG | 96 |
| transmembrane | CCCTCCGACACCCGCTCCCACCATCGCCTCTCAACCTCTTAGTCTTCGCC | |
| (without linker) | CCGAGGCATGCCGACCCGCCGCCGGGGGTGCTGTTCATACGAGGGGCTT | |
| | GGACTTCGCTTGTGATATTTACATTTGGGCTCCGTTGGCGGGTACGTGCG | |
| | GCGTCCTTTTGTTGTCACTCGTTATTACTTTGTATTGTAATCACAGGAATCG | |
| | C | |
| CD8a | FVPVFLPAKPTTTPAPRPPTPAPTIASQPLSLRPEACRPAAGGAVHTRGLDFA | 97 |
| transmembrane | CDIYIWAPLAGTCGVLLLSLVITLYCNHRNR | |
| (without linker) | | |
| CD28 co- | TCAAAGCGGAGTAGGTTGTTGCATTCCGATTACATGAATATGACTCCTCGC | 45 |
| stimulatory | CGGCCTGGGCCGACAAGAAAACATTACCAACCCTATGCCCCCCCACGAGA | |
| | CTTCGCTGCGTACAGGTCC | |
| CD28 co- | SKRSRLLHSDYMNMTPRRPGPTRKHYQPYAPPRDFAAYRS | 46 |
| stimulatory | | |
| 41BB co- | AAACGGGGCAGAAAGAAACTCCTGTATATATTCAAACAACCATTTATGAGA | 43 |
| stimulatory | CCAGTACAAACTACTCAAGAGGAAGATGGCTGTAGCTGCCGATTTCCAGA | |
| | AGAAGAAGAAGGAGGATGTGAACTG | |
| 41BB co- | KRGRKKLLYIFKQPFMRPVQTTQEEDGCSCRFPEEEEGGCEL | 44 |
| stimulatory | | |
| CD3ζ | CGAGTGAAGTTTTCCCGAAGCGCAGACGCTCCGGCATATCAGCAAGGACA | 98 |
| | GAATCAGCTGTATAACGAACTGAATTTGGGACGCCGCGAGGAGTATGACG | |
| | TGCTTGATAAACGCCGGGGGAGAGACCCGGAAATGGGGGGTAAACCCCG | |
| | AAGAAAGAATCCCCAAGAAGGACTCTACAATGAACTCCAGAAGGATAAGAT | |
| | GGCGGAGGCCTACTCAGAAATAGGTATGAAGGGCGAACGACGACGGGGA | |
| | AAAGGTCACGATGGCCTCTACCAAGGGTTGAGTACGGCAACCAAAGATAC | |
| | GTACGATGCACTGCATATGCAGGCCCTGCCTCCCAGA | |
| CD3ζ | RVKFSRSADAPAYQQGQNQLYNELNLGRREEYDVLDKRRGRDPEMGGKPR | 99 |
| | RKNPQEGLYNELQKDKMAEAYSEIGMKGERRRGKGHDGLYQGLSTATKDTY | |
| | DALHMQALPPR | |

TABLE 12
Donor Components
Donor structure: TRAC[LHA]-
EF1a[promoter]-CAR-polyA-TRAC[RHA]

| | | SEQ |
|---|---|---|
| Name | Sequence | ID NO: |
| TRAC-LHA | GAGATGTAAGGAGCTGCTGTGACTTGCTC | 100 |
| | AAGGCCTTATATCGAGTAAACGGTAGTGC | |
| | TGGGGCTTAGACGCAGGTGTTCTGATTTA | |
| | TAGTTCAAAACCTCTATCAATGAGAGAGC | |
| | AATCTCCTGGTAATGTGATAGATTTCCCA | |
| | ACTTAATGCCAACATACCATAAACCTCCC | |
| | ATTCTGCTAATGCCCAGCCTAAGTTGGGG | |
| | AGACCACTCCAGATTCCAAGATGTACAGT | |
| | TTGCTTTGCTGGGCCTTTTTCCCATGCCT | |
| | GCCTTTACTCTGCCAGAGTTATATTGCTG | |
| | GGGTTTTGAAGAAGATCCTATTAAATAAA | |
| | AGAATAAGCAGTATTATTAAGTAGCCCTG | |
| | CATTTCAGGTTTCCTTGAGTGGCAGGCCA | |
| | GGCCTGGCCGTGAACGTTCACTGAAATCA | |
| | TGGCCTCTTGGCCAAGATTGATAGCTTGT | |
| | GCCTGTCCCTGAGTCCCAGTCCATCACGA | |
| | GCAGCTGGTTTCTAAGATGCTATTTCCCG | |
| | TATAAAGCATGAGACCGTGACTTGCCAGC | |
| | CCCACAGAGCCCCGCCCTTGTCCATCACT | |
| | GGCATCTGGACTCCAGCCTGGGTTGGGGC | |
| | AAAGAGGGAAATGAGATCATGTCCTAACC | |
| | CTGATCCTCTTGTCCCACAGATATCCAGA | |
| | ACCCTGACCCTGCCGTGTACCAGCTGAGA | |
| | GACTCTAAATCCAGTGACAAGTCTGTCTG | |
| | CCTATTCACCGATTTTGATTCTCAAACAA | |
| | ATGTGTCACAAAGTAAGGATTCTGATGTG | |
| | TATATCACAGACAAAACTGTGCTAGACAT | |
| | GAGGTCTATGGACTTCA | |
| EF1α | GGCTCCGGTGCCCGTCAGTGGGCAGAGCG | 101 |
| promoter | CACATCGCCCACAGTCCCCGAGAAGTTGG | |
| | GGGGAGGGGTCGGCAATTGAACCGGTGCC | |
| | TAGAGAAGGTGGCGCGGGGTAAACTGGGA | |
| | AAGTGATGTCGTGTACTGGCTCCGCCTTT | |
| | TTCCCGAGGGTGGGGGAGAACCGTATATA | |
| | AGTGCAGTAGTCGCCGTGAACGTTCTTTT | |
| | TCGCAACGGGTTTGCCGCCAGAACACAGG | |
| | TAAGTGCCGTGTGTGGTTCCCGCGGGCCT | |
| | GGCCTCTTTACGGGTTATGGCCCTTGCGT | |
| | GCCTTGAATTACTTCCACTGGCTGCAGTA | |
| | CGTGATTCTTGATCCCGAGCTTCGGGTTG | |
| | GAAGTGGGTGGGAGAGTTCGAGGCCTTGC | |
| | GCTTAAGGAGCCCCTTCGCCTCGTGCTTG | |
| | AGTTGAGGCCTGGCCTGGGCGCTGGGGCC | |
| | GCCGCGTGCGAATCTGGTGGCACCTTCGC | |
| | GCCTGTCTCGCTGCTTTCGATAAGTCTCT | |
| | AGCCATTTAAAATTTTTGATGACCTGCTG | |
| | CGACGCTTTTTTTCTGGCAAGATAGTCTT | |
| | GTAAATGCGGGCCAAGATCTGCACACTGG | |
| | TATTTCGGTTTTTGGGGCCGCGGGCGGCG | |
| | ACGGGGCCCGTGCGTCCCAGCGCACATGT | |
| | TCGGCGAGGCGGGGCCTGCGAGCGCGGCC | |
| | ACCGAGAATCGGACGGGGGTAGTCTCAAG | |
| | CTGGCCGGCCTGCTCTGGTGCCTGGCCTC | |
| | GCGCCGCCGTGTATCGCCCCGCCCTGGGC | |
| | GGCAAGGCTGGCCCGGTCGGCACCAGTTG | |
| | CGTGAGCGGAAAGATGGCCGCTTCCCGGC | |
| | CCTGCTGCAGGGAGCTCAAAATGGAGGAC | |
| | GCGGCGCTCGGGAGAGCGGGCGGGTGAGT | |
| | CACCCACACAAAGGAAAAGGGCCTTTCCG | |
| | TCCTCAGCCGTCGCTTCATGTGACTCCAC | |
| | GGAGTACCGGGCGCCGTCCAGGCACCTCG | |
| | ATTAGTTCTCGAGCTTTTGGAGTACGTCG | |
| | TCTTTAGGTTGGGGGGAGGGGTTTTATGC | |
| | GATGGAGTTTCCCCACACTGAGTGGGTGG | |
| | AGACTGAAGTTAGGCCAGCTTGGCACTTG | |
| | ATGTAATTCTCCTTGGAATTTGCCCTTTT | |
| | TGAGTTTGGATCTTGGTTCATTCTCAAGC | |
| | CTCAGACAGTGGTTCAAAGTTTTTTTCTT | |
| | CCATTTCAGGTGTCGTGA | |
| Synthetic | AATAAAATCGCTATCCATCGAAGATGGAT | 102 |
| poly(A) | GTGTGTTGGTTTTTTGTGTG | |
| signal | | |
| TRAC-RHA | TGGAGCAACAAATCTGACTTTGCATGTGC | 92 |
| | AAACGCCTTCAACAACAGCATTATTCCAG | |
| | AAGACACCTTCTTCCCCAGCCCAGGTAAG | |
| | GGCAGCTTTGGTGCCTTCGCAGGCTGTTT | |
| | CCTTGCTTCAGGAATGGCCAGGTTCTGCC | |
| | CAGAGCTCTGGTCAATGATGTCTAAAACT | |
| | CCTCTGATTGGTGGTCTCGGCCTTATCCA | |
| | TTGCCACCAAAACCCTCTTTTTACTAAGA | |
| | AACAGTGAGCCTTGTTCTGGCAGTCCAGA | |
| | GAATGACACGGGAAAAAAGCAGATGAAGA | |
| | GAAGGTGGCAGGAGAGGGCACGTGGCCCA | |
| | GCCTCAGTCTCTCCAACTGAGTTCCTGCC | |
| | TGCCTGCCTTTGCTCAGACTGTTTGCCCC | |
| | TTACTGCTCTTCTAGGCCTCATTCTAAGC | |
| | CCCTTCTCCAAGTTGCCTCTCCTTATTTC | |
| | TCCCTGTCTGCCAAAAAATCTTTCCCAGC | |
| | TCACTAAGTCAGTCTCACGCAGTCACTCA | |
| | TTAACCCACCAATCACTGATTGTGCCGGC | |
| | ACATGAATGCACCAGGTGTTGAAGTGGAG | |
| | GAATTAAAAAGTCAGATGAGGGGTGTGCC | |
| | CAGAGGAAGCACCATTCTAGTTGGGGGAG | |
| | CCCATCTGTCAGCTGGGAAAAGTCCAAAT | |
| | AACTTCAGATTGGAATGTGTTTTAACTCA | |
| | GGGTTGAGAAAACAGCTACCTTCAGGACA | |
| | AAAGTCAGGGAAGGGCTCTCTGAAGAAAT | |
| | GCTACTTGAAGATACCAGCCCTACCAAGG | |
| | GCAGGGAGAGGACCCTATAGAGGCCTGGG | |
| | ACAGGAGCTCAATGAGAAAGG | |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between the upper and lower ends of the range are specifically contemplated and described herein.

What is claimed is:

1. An engineered T cell comprising a nucleic acid encoding a chimeric antigen receptor (CAR), wherein the CAR comprises an ectodomain that binds specifically to PTK7, wherein the ectodomain of the CAR comprises an anti-PTK7 antibody.


SEQUENCE LISTING (113 sequences)

SEQ ID NO: 1 - DNA, 19 bp, Synthetic construct
  source 1..19: mol_type = other DNA
aagagcaacaaatctgact

SEQ ID NO: 2 - DNA, 58 bp, Synthetic construct
  source 1..58: mol_type = other DNA
aagagcaacagtgctgtgcctggagcaacaaatctgactaagagcaacaaatctgact

SEQ ID NO: 3 - DNA, 52 bp, Synthetic construct
  source 1..52: mol_type = other DNA
aagagcaacagtgctggagcaacaaatctgactaagagcaacaaatctgact

SEQ ID NO: 4 - DNA, 53 bp, Synthetic construct
  source 1..53: mol_type = other DNA
aagagcaacagtgcctggagcaacaaatctgactaagagcaacaaatctgact

SEQ ID NO: 5 - DNA, 38 bp, Synthetic construct
  source 1..38: mol_type = other DNA
aagagcaacagtgctgactaagagcaacaaatctgact

SEQ ID NO: 6 - DNA, 60 bp, Synthetic construct
  source 1..60: mol_type = other DNA
aagagcaacagtgctgtgggcctggagcaacaaatctgactaagagcaacaaatctgact

SEQ ID NO: 7 - DNA, 57 bp, Synthetic construct
  source 1..57: mol_type = other DNA
aagagcaacagtgctggcctggagcaacaaatctgactaagagcaacaaatctgact

SEQ ID NO: 8 - DNA, 60 bp, Synthetic construct
  source 1..60: mol_type = other DNA
aagagcaacagtgctgtgtgcctggagcaacaaatctgactaagagcaacaaatctgact

SEQ ID NO: 9 - DNA, 79 bp, Synthetic construct
  source 1..79: mol_type = other DNA
cgtggccttagctgtgctcgcgctactctctctttctgcctggaggctatccagcgtgagtctctcctaccctcccgct

SEQ ID NO: 10 - DNA, 78 bp, Synthetic construct
  source 1..78: mol_type = other DNA
cgtggccttagctgtgctcgcgctactctctctttcgcctggaggctatccagcgtgagtctctcctaccctcccgct

SEQ ID NO: 11 - DNA, 75 bp, Synthetic construct
  source 1..75: mol_type = other DNA
cgtggccttagctgtgctcgcgctactctctctttctggaggctatccagcgtgagtctctcctaccctcccgct

SEQ ID NO: 12 - DNA, 84 bp, Synthetic construct
  source 1..84: mol_type = other DNA
cgtggccttagctgtgctcgcgctactctctctttctggatagcctggaggctatccagcgtgagtctctcctaccctcccgct

SEQ ID NO: 13 - DNA, 55 bp, Synthetic construct
  source 1..55: mol_type = other DNA
cgtggccttagctgtgctcgcgctatccagcgtgagtctctcctaccctcccgct

SEQ ID NO: 14 - DNA, 82 bp, Synthetic construct
  source 1..82: mol_type = other DNA
cgtggccttagctgtgctcgcgctactctctctttctgtggcctggaggctatccagcgtgagtctctcctaccctcccgct

SEQ ID NO: 15 - RNA, 100 nt, Synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 16 - RNA, 96 nt, Synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 17 - RNA, 114 nt, Synthetic construct
  source 1..114: mol_type = other RNA
nnnnnnnnnnnnnnnnnnnnnnnnnnnnnngttttagagctagaaatagcaagttaaaataaggctagtccgttatcaacttgaaaaagtggcaccgagtcggtgctttttttt

SEQ ID NO: 18 - RNA, 100 nt, Synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 19 - RNA, 20 nt, Synthetic construct
  source 1..20: mol_type = other RNA
agagcaacagtgctgtggcc

SEQ ID NO: 20 - RNA, 100 nt, Synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 21 - RNA, 20 nt, Synthetic construct
  source 1..20: mol_type = other RNA
gctactctctctttctggcc

SEQ ID NO: 22 - RNA, 100 nt, Synthetic construct
  source 1..100: mol_type = other RNA
ctgcagcttctccaacacatgttttagagctagaaatagcaagttaaaataaggctagtccgttatcaacttgaaaaagtggcaccgagtcggtgctttt

SEQ ID NO: 23 - RNA, 20 nt, Synthetic construct
  source 1..20: mol_type = other RNA
ctgcagcttctccaacacat

SEQ ID NO: 24 - RNA, 100 nt, Synthetic construct
  source 1..100: mol_type = other RNA
gctttggtcccattggtcgcgttttagagctagaaatagcaagttaaaataaggctagtccgttatcaacttgaaaaagtggcaccgagtcggtgctttt

SEQ ID NO: 25 - RNA, 20 nt, Synthetic construct
  source 1..20: mol_type = other RNA
gctttggtcccattggtcgc

SEQ ID NO: 26 - RNA, 100 nt, Synthetic construct
  source 1..100: mol_type = other RNA
gcccgcaggacgcacccatagttttagagctagaaatagcaagttaaaataaggctagtccgttatcaacttgaaaaagtggcaccgagtcggtgctttt

SEQ ID NO: 27 - RNA, 20 nt, Synthetic construct
  source 1..20: mol_type = other RNA
gcccgcaggacgcacccata

SEQ ID NO: 28 - RNA, 100 nt, Synthetic construct; identical to SEQ ID NO: 18
  source 1..100: mol_type = other RNA
  modified_base 1..3: mod_base = OTHER; note = 2'-O-methyl phosphorothioate
  modified_base 97..99: mod_base = OTHER; note = 2'-O-methyl phosphorothioate

SEQ ID NO: 29 - RNA, 20 nt, Synthetic construct
  source 1..20: mol_type = other RNA
  modified_base 1..3: mod_base = OTHER; note = 2'-O-methyl phosphorothioate
agagcaacagtgctgtggcc

SEQ ID NO: 30 - RNA, 97 nt, Synthetic construct
  source 1..97: mol_type = other RNA
  modified_base 94..96: mod_base = OTHER; note = 2'-O-methyl phosphorothioate
actctctctttctggccgttttagagctagaaatagcaagttaaaataaggctagtccgttatcaacttgaaaaagtggcaccgagtcggtgctttt

SEQ ID NO: 31 - RNA, 20 nt, Synthetic construct
  source 1..20: mol_type = other RNA
  modified_base 1..3: mod_base = OTHER; note = 2'-O-methyl phosphorothioate
gctactctctctttctggcc

SEQ ID NO: 32 - RNA, 100 nt, Synthetic construct; identical to SEQ ID NO: 22
  source 1..100: mol_type = other RNA
  modified_base 1..3: mod_base = OTHER; note = 2'-O-methyl phosphorothioate
  modified_base 97..99: mod_base = OTHER; note = 2'-O-methyl phosphorothioate

SEQ ID NO: 33 - RNA, 20 nt, Synthetic construct
  source 1..20: mol_type = other RNA
  modified_base 1..3: mod_base = OTHER; note = 2'-O-methyl phosphorothioate
ctgcagcttctccaacacat

SEQ ID NO: 34 - RNA, 100 nt, Synthetic construct; identical to SEQ ID NO: 24
  source 1..100: mol_type = other RNA
  modified_base 1..3: mod_base = OTHER; note = 2'-O-methyl phosphorothioate
  modified_base 97..99: mod_base = OTHER; note = 2'-O-methyl phosphorothioate

SEQ ID NO: 35 - RNA, 20 nt, Synthetic construct
  source 1..20: mol_type = other RNA
  modified_base 1..3: mod_base = OTHER; note = 2'-O-methyl phosphorothioate
gctttggtcccattggtcgc

SEQ ID NO: 36 - RNA, 100 nt, Synthetic construct; identical to SEQ ID NO: 26
  source 1..100: mol_type = other RNA
  modified_base 97..99: mod_base = OTHER; note = 2'-O-methyl phosphorothioate
  modified_base 1..3: mod_base = OTHER; note = 2'-O-methyl phosphorothioate

SEQ ID NO: 37 - RNA, 20 nt, Synthetic construct
  source 1..20: mol_type = other RNA
  modified_base 1..3: mod_base = OTHER; note = 2'-O-methyl phosphorothioate
gcccgcaggacgcacccata

SEQ ID NO: 38 - DNA, 23 bp, Synthetic construct
  source 1..23: mol_type = other DNA
gctttggtcccattggtcgcggg

SEQ ID NO: 39 - DNA, 23 bp, Synthetic construct
  source 1..23: mol_type = other DNA
gcccgcaggacgcacccataggg

SEQ ID NO: 40 - DNA, 23 bp, Synthetic construct
  source 1..23: mol_type = other DNA
agagcaacagtgctgtggcctgg

SEQ ID NO: 41 - DNA, 23 bp, Synthetic construct
  source 1..23: mol_type = other DNA
gctactctctctttctggcctgg

SEQ ID NO: 42 - DNA, 23 bp, Synthetic construct
  source 1..23: mol_type = other DNA
ctgcagcttctccaacacatcgg

SEQ ID NO: 43 - DNA, 126 bp, Synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 44 - protein, 42 aa, Synthetic construct
KRGRKKLLYIFKQPFMRPVQTTQEEDGCSCRFPEEEEGGCEL

SEQ ID NO: 45 - DNA, 120 bp, Synthetic construct
  source 1..120: mol_type = other DNA
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 46 - protein, 40 aa, Synthetic construct
SKRSRLLHSDYMNMTPRRPGPTRKHYQPYAPPRDFAAYRS

SEQ ID NO: 47 - DNA, 336 bp, Synthetic construct
  source 1..336: mol_type = other DNA
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 48 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 1530 bp, Synthetic construct
  source 1..1530: mol_type = other DNA
atggcgctgccggtgaccgcgctgctgctgccgctggcgctgctgctgcatgcggcgcgcccgcaggtgcagctggtggaaagcggcggcggcgtggtgcagccgggccgcagcctgcgcctgagctgcgcggcgagcggctttacctttagcagctatggcatgcattgggtgcgccaggcgccgggcaaaggcctggaatgggtggcggtgatttgggatgatggcagcaacaaatattatgtggatagcgtgaaaggccgctttaccattagccgcgataacagcaaaaacaccctgtatctgcagatgaacagcctgcgcgcggaagataccgcggtgtattattgcgcgcgcgatgattattatggcagcggcagctttaacagctattatggcaccgatgtgtggggccagggcaccaccgtgaccgtgagcagcggcggcggcggcagcggcggcggcggcagcggcggcggcggcagcgaaattgtgctgacccagagcccggcgaccctgagcctgagcccgggcgaacgcgcgaccctgagctgccgcgcgagccagagcgtgagcatttatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgtttacctttggcccgggcaccaaagtggatattaaaagcgcggcggcgtttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgcagcaaacgcagccgcctgctgcatagcgattatatgaacatgaccccgcgccgcccgggcccgacccgcaaacattatcagccgtatgcgccgccgcgcgattttgcggcgtatcgcagccgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 50 - protein, 510 aa, Synthetic construct
MALPVTALLLPLALLLHAARPQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSGGGGSGGGGSGGGGSEIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 1536 bp, Synthetic construct
  source 1..1536: mol_type = other DNA
atggcgctgccggtgaccgcgctgctgctgccgctggcgctgctgctgcatgcggcgcgcccgcaggtgcagctggtggaaagcggcggcggcgtggtgcagccgggccgcagcctgcgcctgagctgcgcggcgagcggctttacctttagcagctatggcatgcattgggtgcgccaggcgccgggcaaaggcctggaatgggtggcggtgatttgggatgatggcagcaacaaatattatgtggatagcgtgaaaggccgctttaccattagccgcgataacagcaaaaacaccctgtatctgcagatgaacagcctgcgcgcggaagataccgcggtgtattattgcgcgcgcgatgattattatggcagcggcagctttaacagctattatggcaccgatgtgtggggccagggcaccaccgtgaccgtgagcagcggcggcggcggcagcggcggcggcggcagcggcggcggcggcagcgaaattgtgctgacccagagcccggcgaccctgagcctgagcccgggcgaacgcgcgaccctgagctgccgcgcgagccagagcgtgagcatttatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgtttacctttggcccgggcaccaaagtggatattaaaagcgcggcggcgtttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgcaaacgcggccgcaaaaaactgctgtatatttttaaacagccgtttatgcgcccggtgcagaccacccaggaagaagatggctgcagctgccgctttccggaagaagaagaaggcggctgcgaactgcgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 52 - protein, 512 aa, Synthetic construct
MALPVTALLLPLALLLHAARPQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSGGGGSGGGGSGGGGSEIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 747 bp, Synthetic construct
  source 1..747: mol_type = other DNA
caggtgcagctggtggaaagcggcggcggcgtggtgcagccgggccgcagcctgcgcctgagctgcgcggcgagcggctttacctttagcagctatggcatgcattgggtgcgccaggcgccgggcaaaggcctggaatgggtggcggtgatttgggatgatggcagcaacaaatattatgtggatagcgtgaaaggccgctttaccattagccgcgataacagcaaaaacaccctgtatctgcagatgaacagcctgcgcgcggaagataccgcggtgtattattgcgcgcgcgatgattattatggcagcggcagctttaacagctattatggcaccgatgtgtggggccagggcaccaccgtgaccgtgagcagcggcggcggcggcagcggcggcggcggcagcggcggcggcggcagcgaaattgtgctgacccagagcccggcgaccctgagcctgagcccgggcgaacgcgcgaccctgagctgccgcgcgagccagagcgtgagcatttatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgtttacctttggcccgggcaccaaagtggatattaaa

SEQ ID NO: 54 - protein, 249 aa, Synthetic construct
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSGGGGSGGGGSGGGGSEIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIK

SEQ ID NO: 55 - protein, 126 aa, Synthetic construct
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 56 - protein, 108 aa, Synthetic construct
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIK

SEQ ID NO: 57 - protein, 5 aa, Synthetic construct
SYGMH

SEQ ID NO: 58 - protein, 17 aa, Synthetic construct
VIWDDGSNKYYVDSVKG

SEQ ID NO: 59 - protein, 17 aa, Synthetic construct
DDYYGSGSFNSYYGTDV

SEQ ID NO: 60 - protein, 11 aa, Synthetic construct
RASQSVSIYLA

SEQ ID NO: 61 - protein, 7 aa, Synthetic construct
DASNRAT

SEQ ID NO: 62 - protein, 10 aa, Synthetic construct
QQRSNWPPFT

SEQ ID NO: 63 - DNA, 4370 bp, Synthetic construct
  source 1..4370: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtggagagcggcggaggagtggtgcaacccggaaggtccctgaggctctcctgtgccgccagcggcttcaccttctccagctacggtatgcactgggtgagacaagcccccggaaagggcctcgagtgggtggccgtgatctgggatgatggctccaacaagtactacgtggacagcgtcaagggcagattcaccatcagcagggacaacagcaagaacaccctgtacctgcagatgaactccctgagagccgaagacaccgccgtgtactattgtgccagggacgactactatggctccggctccttcaatagctactatggcaccgacgtgtggggccagggcaccacagtgacagtgagcagcggcggaggaggatccggaggaggaggaagcggaggaggaggaagcgagatcgtgctgacacagtcccccgctaccctgagcctgagccccggcgagagagctaccctgagctgcagagccagccagagcgtctccatctacctggcctggtaccagcagaagcctggccaggcccctagactgctgatctacgacgccagcaacagggccaccggcattcctgccagattcagcggctccggctccggcaccgatttcacactgaccatcagctccctggagcctgaggacttcgccgtgtattactgccagcagaggagcaactggcccccctttaccttcggccccggcaccaaggtcgacatcaagagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 64 - DNA, 4376 bp, Synthetic construct
  source 1..4376: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtggagagcggcggaggagtggtgcaacccggaaggtccctgaggctctcctgtgccgccagcggcttcaccttctccagctacggtatgcactgggtgagacaagcccccggaaagggcctcgagtgggtggccgtgatctgggatgatggctccaacaagtactacgtggacagcgtcaagggcagattcaccatcagcagggacaacagcaagaacaccctgtacctgcagatgaactccctgagagccgaagacaccgccgtgtactattgtgccagggacgactactatggctccggctccttcaatagctactatggcaccgacgtgtggggccagggcaccacagtgacagtgagcagcggcggaggaggatccggaggaggaggaagcggaggaggaggaagcgagatcgtgctgacacagtcccccgctaccctgagcctgagccccggcgagagagctaccctgagctgcagagccagccagagcgtctccatctacctggcctggtaccagcagaagcctggccaggcccctagactgctgatctacgacgccagcaacagggccaccggcattcctgccagattcagcggctccggctccggcaccgatttcacactgaccatcagctccctggagcctgaggacttcgccgtgtattactgccagcagaggagcaactggcccccctttaccttcggccccggcaccaaggtcgacatcaagagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 65 - DNA, 1527 bp, Synthetic construct
  source 1..1527: mol_type = other DNA
atggcgctgccggtgaccgcgctgctgctgccgctggcgctgctgctgcatgcggcgcgcccggaaattgtgctgacccagagcccggcgaccctgagcctgagcccgggcgaacgcgcgaccctgagctgccgcgcgagccagagcgtgagcatttatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgtttacctttggcccgggcaccaaagtggatattaaaggcggcggcggcagcggcggcggcggcagcggcggcggcggcagccaggtgcagctggtggaaagcggcggcggcgtggtgcagccgggccgcagcctgcgcctgagctgcgcggcgagcggctttacctttagcagctatggcatgcattgggtgcgccaggcgccgggcaaaggcctggaatgggtggcggtgatttgggatgatggcagcaacaaatattatgtggatagcgtgaaaggccgctttaccattagccgcgataacagcaaaaacaccctgtatctgcagatgaacagcctgcgcgcggaagataccgcggtgtattattgcgcgcgcgatgattattatggcagcggcagctttaacagctattatggcaccgatgtgtggggccagggcaccaccgtgaccgtgagcagcgcggcggcgtttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgcagcaaacgcagccgcctgctgcatagcgattatatgaacatgaccccgcgccgcccgggcccgacccgcaaacattatcagccgtatgcgccgccgcgcgattttgcggcgtatcgcagccgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 66 - protein, 509 aa, Synthetic construct
MALPVTALLLPLALLLHAARPEIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIKGGGGSGGGGSGGGGSQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 747 bp, Synthetic construct
  source 1..747: mol_type = other DNA
gaaattgtgctgacccagagcccggcgaccctgagcctgagcccgggcgaacgcgcgaccctgagctgccgcgcgagccagagcgtgagcatttatctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagcgcagcaactggccgccgtttacctttggcccgggcaccaaagtggatattaaaggcggcggcggcagcggcggcggcggcagcggcggcggcggcagccaggtgcagctggtggaaagcggcggcggcgtggtgcagccgggccgcagcctgcgcctgagctgcgcggcgagcggctttacctttagcagctatggcatgcattgggtgcgccaggcgccgggcaaaggcctggaatgggtggcggtgatttgggatgatggcagcaacaaatattatgtggatagcgtgaaaggccgctttaccattagccgcgataacagcaaaaacaccctgtatctgcagatgaacagcctgcgcgcggaagataccgcggtgtattattgcgcgcgcgatgattattatggcagcggcagctttaacagctattatggcaccgatgtgtggggccagggcaccaccgtgaccgtgagcagc

SEQ ID NO: 68 - protein, 249 aa, Synthetic construct
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIKGGGGSGGGGSGGGGSQVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSS

SEQ ID NO: 69 - protein, 126 aa, Synthetic construct; identical to SEQ ID NO: 55

SEQ ID NO: 70 - protein, 108 aa, Synthetic construct; identical to SEQ ID NO: 56

SEQ ID NO: 71 - DNA, 4370 bp, Synthetic construct
  source 1..4370: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggagatcgtgctgacccagagccctgccacactgagcctgagccccggagagagggctaccctgagctgcagggcctcccagtccgtgagcatctacctggcctggtaccagcagaaacctggccaggcccccaggctgctgatctacgacgccagcaatagggccaccggaatccctgccaggtttagcggctccggaagcggcaccgacttcaccctgaccatctcctccctggagcccgaggatttcgccgtgtactactgccagcagaggtccaactggcctccctttaccttcggccccggcaccaaggtggatattaagggcggcggcggatccggaggaggaggcagcggaggaggaggaagccaggtgcaactggtggagtccggcggaggcgtggtgcaacctggcagaagcctgaggctgagctgtgccgccagcggcttcaccttcagcagctacggtatgcactgggtgaggcaggctcccggaaagggcctggaatgggtggccgtgatctgggacgacggctccaacaagtactacgtggactccgtgaagggcaggttcaccatcagcagggacaactccaagaacacactgtacctgcagatgaacagcctgagggccgaggataccgctgtgtattactgcgccagggacgattactacggcagcggcagcttcaattcctactacggaaccgacgtctggggccagggaaccaccgtgaccgtgagcagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 72 - DNA, 1488 bp, Synthetic construct
  source 1..1488: mol_type = other DNA
atggcgctgccggtgaccgcgctgctgctgccgctggcgctgctgctgcatgcggcgcgcccggaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagcccgatgtatacctttggccagggcaccaaactggaaattaaaggcggcggcggcagcggcggcggcggcagcggcggcggcggcagcgaagtgcagctggtgcagagcggcggcggcctggtgcatccgggcggcagcctgcgcctgagctgcgcgggcagcggctttacctttagcacctatctgatgtattgggtgcgccaggcgccgggcaaaaccctggaatgggtgagcgcgattggcagcggcggcgatacctattatgcggatagcgtgaaaggccgctttaccattagccgcgataacgcgaaaaacagcctgtatctgcagatgaacagcctgcgcgcggaagatatggcggtgtattattgcgcgcgcggcctgggctattggggccagggcaccctggtgaccgtgagcagcgcggcggcgtttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgcagcaaacgcagccgcctgctgcatagcgattatatgaacatgaccccgcgccgcccgggcccgacccgcaaacattatcagccgtatgcgccgccgcgcgattttgcggcgtatcgcagccgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 73 - protein, 496 aa, Synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIKGGGGSGGGGSGGGGSEVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - skipped

SEQ ID NO: 75 - protein, 236 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIKGGGGSGGGGSGGGGSEVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 76 - protein, 112 aa, Synthetic construct
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSS

SEQ ID NO: 77 - protein, 109 aa, Synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 78 - DNA, 4331 bp, Synthetic construct
  source 1..4331: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggagatcgtgctgacccagagccccggaacactgagcctgtcccccggagaaagagccacactgtcctgcagggccagccagagcgtgagcagctcctacctggcctggtaccagcagaagcctggacaggcccccaggctgctgatttacggcgccagcagcagggccaccggcatccccgacagattcagcggatccggcagcggcaccgacttcaccctgaccatcagcaggctggagcccgaggacttcgctgtgtactactgccagcagtacggcagcagccccatgtacaccttcggccagggcaccaagctggagatcaagggaggaggaggatccggaggaggcggaagcggaggaggaggaagcgaggtgcagctggtgcagagcggcggaggactggtgcatcccggaggatccctgagactgagctgtgccggcagcggattcacattctccacctacctgatgtactgggtgaggcaggcccctggcaagaccctggagtgggtgtccgccattggctccggcggagacacctattatgccgactccgtcaagggcaggttcaccatcagcagagacaacgccaagaactccctgtacctgcagatgaacagcctgagggccgaggatatggctgtgtactattgcgctaggggcctgggatactggggccagggaaccctggtgaccgtgagctccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 79 - DNA, 1491 bp, Synthetic construct
  source 1..1491: mol_type = other DNA
atggcgctgccggtgaccgcgctgctgctgccgctggcgctgctgctgcatgcggcgcgcccggaagtgcagctggtgcagagcggcggcggcctggtgcatccgggcggcagcctgcgcctgagctgcgcgggcagcggctttacctttagcacctatctgatgtattgggtgcgccaggcgccgggcaaaaccctggaatgggtgagcgcgattggcagcggcggcgatacctattatgcggatagcgtgaaaggccgctttaccattagccgcgataacgcgaaaaacagcctgtatctgcagatgaacagcctgcgcgcggaagatatggcggtgtattattgcgcgcgcggcctgggctattggggccagggcaccctggtgaccgtgagcagcggcggcggcggcagcggcggcggcggcagcggcggcggcggcagcgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagcccgatgtatacctttggccagggcaccaaactggaaattaaaagcgcggcggcgtttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgcagcaaacgcagccgcctgctgcatagcgattatatgaacatgaccccgcgccgcccgggcccgacccgcaaacattatcagccgtatgcgccgccgcgcgattttgcggcgtatcgcagccgcgtgaaatttagccgcagcgcggatgcgccggcgtatcagcagggccagaaccagctgtataacgaactgaacctgggccgccgcgaagaatatgatgtgctggataaacgccgcggccgcgatccggaaatgggcggcaaaccgcgccgcaaaaacccgcaggaaggcctgtataacgaactgcagaaagataaaatggcggaagcgtatagcgaaattggcatgaaaggcgaacgccgccgcggcaaaggccatgatggcctgtatcagggcctgagcaccgcgaccaaagatacctatgatgcgctgcatatgcaggcgctgccgccgcgc

SEQ ID NO: 80 - protein, 497 aa, Synthetic construct
MALPVTALLLPLALLLHAARPEVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - DNA, 708 bp, Synthetic construct
  source 1..708: mol_type = other DNA
gaagtgcagctggtgcagagcggcggcggcctggtgcatccgggcggcagcctgcgcctgagctgcgcgggcagcggctttacctttagcacctatctgatgtattgggtgcgccaggcgccgggcaaaaccctggaatgggtgagcgcgattggcagcggcggcgatacctattatgcggatagcgtgaaaggccgctttaccattagccgcgataacgcgaaaaacagcctgtatctgcagatgaacagcctgcgcgcggaagatatggcggtgtattattgcgcgcgcggcctgggctattggggccagggcaccctggtgaccgtgagcagcggcggcggcggcagcggcggcggcggcagcggcggcggcggcagcgaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagcccgatgtatacctttggccagggcaccaaactggaaattaaa

SEQ ID NO: 82 - protein, 236 aa, Synthetic construct
EVQLVQSGGGLVHPGGSLRLSCAGSGFTFSTYLMYWVRQAPGKTLEWVSAIGSGGDTYYADSVKGRFTISRDNAKNSLYLQMNSLRAEDMAVYYCARGLGYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPMYTFGQGTKLEIK

SEQ ID NO: 83 - protein, 112 aa, Synthetic construct; identical to SEQ ID NO: 76

SEQ ID NO: 84 - protein, 109 aa, Synthetic construct; identical to SEQ ID NO: 77

SEQ ID NO: 85 - protein, 5 aa, Synthetic construct
TYLMY

SEQ ID NO: 86 - protein, 16 aa, Synthetic construct
AIGSGGDTYYADSVKG

SEQ ID NO: 87 - protein, 4 aa, Synthetic construct
GLGY

SEQ ID NO: 88 - protein, 12 aa, Synthetic construct
RASQSVSSSYLA

SEQ ID NO: 89 - protein, 7 aa, Synthetic construct
GASSRAT

SEQ ID NO: 90 - protein, 10 aa, Synthetic construct
QQYGSSPMYT

SEQ ID NO: 91 - DNA, 4331 bp, Synthetic construct
  source 1..4331: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggaggtccagctggtgcagagcggaggcggactggtgcatcctggaggctccctgagactgtcctgtgccggcagcggcttcaccttcagcacctacctgatgtactgggtgagacaggcccccggcaaaaccctggagtgggtgagcgctatcggcagcggcggagacacatactacgccgacagcgtgaagggcaggttcaccatcagcagggacaacgccaagaactccctgtacctgcagatgaactccctgagggctgaggacatggccgtgtactactgcgctagaggcctgggctactggggacagggcacactggtgacagtgagcagcggaggcggcggcagcggaggcggcggcagcggcggcggaggcagcgagatcgtgctgacacagagccctggcaccctgtccctgtcccctggcgaaagggccaccctgagctgtagggccagccagtccgtgagcagcagctatctggcctggtaccagcagaaacccggccaggcccctagactgctgatctacggcgcctcctccagagccaccggaatccccgatagattcagcggctccggcagcggcaccgatttcacactgaccatcagcaggctggagcccgaggacttcgccgtgtattactgccagcagtacggcagcagccctatgtacacattcggccagggcaccaagctggagatcaagagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 92 - DNA, 804 bp, Synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 93 - protein, 21 aa, Synthetic construct
MALPVTALLLPLALLLHAARP

SEQ ID NO: 94 - DNA, 261 bp, Synthetic construct
  source 1..261: mol_type = other DNA
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 95 - protein, 88 aa, Synthetic construct
SAAAFVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 96 - DNA, 252 bp, Synthetic construct
  source 1..252: mol_type = other DNA
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 97 - protein, 84 aa, Synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 98 - DNA, 336 bp, Synthetic construct; identical to SEQ ID NO: 47
  source 1..336: mol_type = other DNA

SEQ ID NO: 99 - protein, 112 aa, Synthetic construct; identical to SEQ ID NO: 48

SEQ ID NO: 100 - DNA, 800 bp, Synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 101 - DNA, 1178 bp, Synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 102 - DNA, 49 bp, Synthetic construct
  source 1..49: mol_type = other DNA
aataaaatcgctatccatcgaagatggatgtgtgttggttttttgtgtg

SEQ ID NO: 103 - skipped

SEQ ID NO: 104 - DNA, 20 bp, Synthetic construct
  source 1..20: mol_type = other DNA
agagcaacagtgctgtggcc

SEQ ID NO: 105 - RNA, 20 nt, Synthetic construct
  source 1..20: mol_type = other RNA
agagcaacagtgctgtggcc

SEQ ID NO: 106 - protein, 22 aa, Synthetic construct
MLLLVTSLLLCELPHPAFLLIP

SEQ ID NO: 107 - protein, 84 aa, Synthetic construct; identical to SEQ ID NO: 97

SEQ ID NO: 108 - protein, 456 aa, Synthetic construct
QVQLVESGGGVVQPGRSLRLSCAASGFTFSSYGMHWVRQAPGKGLEWVAVIWDDGSNKYYVDSVKGRFTISRDNSKNTLYLQMNSLRAEDTAVYYCARDDYYGSGSFNSYYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 215 aa, Synthetic construct
EIVLTQSPATLSLSPGERATLSCRASQSVSIYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - RNA, 100 nt, Synthetic construct
  source 1..100: mol_type = other RNA
ccgccgcgatgggagctgcggttttagagctagaaatagcaagttaaaataaggctagtccgttatcaacttgaaaaagtggcaccgagtcggtgctttt

SEQ ID NO: 111 - RNA, 100 nt, Synthetic construct; identical to SEQ ID NO: 110
  source 1..100: mol_type = other RNA
  modified_base 1..3: mod_base = OTHER; note = 2'-O-methyl phosphorothioate
  modified_base 97..99: mod_base = OTHER; note = 2'-O-methyl phosphorothioate

SEQ ID NO: 112 - DNA, 1530 bp, Synthetic construct
  source 1..1530: mol_type = other DNA
atggcgcttccggtgacagcactgctcctccccttggcgctgttgctccacgcagcaaggccgcaggtgcagctggtggagagcggcggaggagtggtgcaacccggaaggtccctgaggctctcctgtgccgccagcggcttcaccttctccagctacggtatgcactgggtgagacaagcccccggaaagggcctcgagtgggtggccgtgatctgggatgatggctccaacaagtactacgtggacagcgtcaagggcagattcaccatcagcagggacaacagcaagaacaccctgtacctgcagatgaactccctgagagccgaagacaccgccgtgtactattgtgccagggacgactactatggctccggctccttcaatagctactatggcaccgacgtgtggggccagggcaccacagtgacagtgagcagcggcggaggaggatccggaggaggaggaagcggaggaggaggaagcgagatcgtgctgacacagtcccccgctaccctgagcctgagccccggcgagagagctaccctgagctgcagagccagccagagcgtctccatctacctggcctggtaccagcagaagcctggccaggcccctagactgctgatctacgacgccagcaacagggccaccggcattcctgccagattcagcggctccggctccggcaccgatttcacactgaccatcagctccctggagcctgaggacttcgccgtgtattactgccagcagaggagcaactggcccccctttaccttcggccccggcaccaaggtcgacatcaagagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 113 - DNA, 747 bp, Synthetic construct; identical to SEQ ID NO: 53
  source 1..747: mol_type = other DNA